(19) United States
(12) Patent Application Publication
(10) Pub. No.: US 2024/0417805 A1
(43) Pub. Date: Dec. 19, 2024

(54) CANCER NEOANTIGENS

(71) Applicant: CureVac Netherlands B.V., Amsterdam (NL)

(72) Inventors: Ronald Hans Anton PLASTERK, Amsterdam (NL); Wigard Pieter KLOOSTERMAN, Bunnik (NL); Michael Vincent MARTIN, Amsterdam (NL)

(21) Appl. No.: 18/703,444

(22) PCT Filed: Oct. 21, 2022
(86) PCT No.: PCT/NL2022/050597

§ 371 Date: Apr. 22, 2024

(30) Foreign Application Priority Data
Oct. 21, 2021 (NL) 2029480

(51) Int. Cl. C12Q 1/6886; A61K 39/00

(52) U.S. Cl. CPC C12Q 1/6886; A61K 39/4611; A61K 39/464401; A61K 2039/53; A61K 2039/55555

(57) ABSTRACT

The invention relates to the field of cancer. In particular, it relates to the field of immune system directed approaches for tumor treatment, reduction and control. Some aspects of the invention relate to the identification of tumor specific neoantigens, such as those resulting from frameshift mutations, DNA rearrangements, or splicing mutations. Such neoantigens are useful for developing tumor treatments, such as vaccines or cellular immunotherapies and other means of stimulating a neoantigen specific immune response against a tumor in individuals.

### FIELD OF THE INVENTION

The invention relates to the field of cancer. In particular, it relates to the field of immune system directed approaches for tumor treatment, reduction and control. Some aspects of the invention relate to the identification of tumor specific neoantigens, such as those resulting from frameshift mutations, DNA rearrangements, and splicing mutations. Such neoantigens are useful for developing tumor treatments, such as vaccines or cellular immunotherapies and other means of stimulating a neoantigen specific immune response against a tumor in individuals.

### BACKGROUND OF THE INVENTION

There are a number of different existing cancer therapies, including ablation techniques (e.g., surgical procedures and radiation) and chemical techniques (e.g., pharmaceutical agents and antibodies), and various combinations of such techniques. Despite intensive research such therapies are still frequently associated with serious risk, adverse or toxic side effects, as well as varying efficacy.

There is a growing interest in cancer therapies that aim to target cancer cells with a patient's own immune system (such as cancer vaccines or checkpoint inhibitors, or T-cell based immunotherapy). Such therapies may indeed eliminate some of the known disadvantages of existing therapies or be used in addition to the existing therapies for additional therapeutic effect. Cancer vaccines or immunogenic compositions intended to treat an existing cancer by strengthening the body's natural defenses against the cancer and based on tumor-specific neoantigens hold great promise as personalized cancer immunotherapy. Evidence shows that such neoantigen-based vaccination can elicit T-cell responses and can cause tumor regression in patients.

Typically, the immunogenic compositions/vaccines are composed of tumor antigens (antigenic peptides or nucleic acids encoding them) and may include immune stimulatory molecules like cytokines that work together to induce antigen-specific cytotoxic T-cells that target and destroy tumor cells. Many reports describe vaccination based on somatic SNVs (Single Nucleotide Variants) that lead to single amino acid changes in proteins, and hence encode new antigens (neoantigens) that are specific to the tumor. On average 95% of all protein-altering coding somatic mutations in the ORFeome (i.e. the entire collection of all Open Reading Frame sequences in the genome) of tumors (excluding synonymous or truncating SNVs) are missense SNVs (Single Nucleotide Variants), as based on the tumor mutation reports available for the TCGA database. Neoantigens have also been described in e.g., WO2016/191545, US2016/331822 and WO2021172990.

Much of the research in recent years has focused on the prediction (either in silico or by experimental analysis) of which of these many mutations would make for the best neoantigen to use as a vaccine (Schumacher, T. N., Scheper, W. & Kvistborg, P. Cancer Neoantigens. Annu. Rev. Immunol. 37, 173-200 (2019)). Recent experimental estimates suggest that for about 1.6% of gene products encoded by somatic nonsynonymous single nucleotide variations mutation-specific T-cells can be found in cancer patient samples (Parkhurst et al. Cancer Discov Aug. 1, 2019 9(8) 1022-1035). On average (but widely differing per tumor type, see e.g. Priestley, P. et al. Pan-cancer whole genome analyses of metastatic solid tumors. Nature volume 575, 210-216, 2019) a tumor ORFeome contains 200 missense mutations, and the practical limit of the number of peptide vaccines that can be applied to any patient has been set anywhere between 5 and 20, so that at max only a few percent of the neoantigens caused by missense mutations can be used for vaccination (see, e.g., Keskin, D. B. et al. Neoantigen vaccine generates intratumoral T cell responses in phase Ib glioblastoma trial. Nature 565, 234-239 (2019) and Ott, P. A. et al. An immunogenic personal neoantigen vaccine for patients with melanoma. Nature 547, 217-221 (2017). Therefore, the choice of the “best” SNVs is indeed crucial. In this choice it is usually considered that the peptide containing the SNV-neoantigen needs to be presented by the MHC, so that prediction of the presentation by the MHC-type of the patient is essential. For vaccine technologies other than peptides, such as DNA or RNA encoded vaccines, the number of SNVs to be included in a vaccine may be higher than 5-20, but in none of current approaches is the complete set or even the majority of all neoantigenic amino acid sequences included (Hilf, N. et al. Actively personalized vaccination trial for newly diagnosed glioblastoma. Nature 565, 240-245 (2019)).

Accordingly, there is a need for improved methods and compositions for providing subject-specific immunogenic compositions/cancer vaccines. In particular, there is a need for cancer immunogenic compositions that do not rely on predicting which individual neoantigens will be most effective in vivo. One object of the present disclosure is to take the guesswork out of neoantigen selection by identifying a large part of the tumor antigenicity. A further object of the present disclosure is to provide methods for uncovering neoantigens resulting from splicing mutations and/or neoantigens resulting from mutations of stop codons and the use of said neoantigens as immunogenic compositions/cancer vaccines.

### SUMMARY OF THE INVENTION

The disclosure provides the following preferred embodiments.

In one aspect, the disclosure provides a method for identifying neoantigen sequences, said method comprising:
i) performing whole genome sequencing of a tumor sample and a healthy sample from an individual,
ii) performing long-read RNA sequencing on RNA or long-read sequencing on the corresponding cDNA from at least one tumor sample, preferably wherein the RNA is poly-(A) selected mRNA and/or 5′ cap containing mRNA;
iii) identifying somatic genomic changes in nucleic acid sequences from at least one tumor sample from an individual, said step comprising determining the presence of single nucleotide variants (SNVs), indels, and structural variants that result in tumor specific open reading frames,
   wherein said step comprises:
      determining the presence of cis-splicing mutations that result in tumor specific open reading frames;
      determining the presence of intragenic frameshift mutations in polypeptide encoding sequences, wherein the mutation results in a tumor specific open reading frame,
      determining the presence of DNA rearrangements resulting in new junctions of DNA sequences, wherein the new DNA junction results in a tumor specific open reading frame, and
      determining the presence of a mutation in a stop codon, wherein the mutation results in a tumor specific open reading frame;
iv) determining the predicted amino acid sequences encoded by the tumor specific open reading frames, and
v) selecting, as neoantigen peptide sequences, amino acid sequences comprising at least 9 amino acids, wherein the neoantigen peptide sequences comprise at least four contiguous amino acids encoded by the tumor specific open reading frames.

In some embodiments, step i) comprises performing short-read whole genome sequencing. In some embodiments, step i) comprises performing long-read whole genome sequencing, instead of or in addition to short-read sequencing, of a tumor sample and a healthy sample from the individual. Preferably, the RNA sequencing is performed using long-read direct RNA sequencing, preferably Nanopore sequencing, or long-read cDNA sequencing. Preferably, the method further comprises performing short-read RNA sequencing on RNA or short-read sequencing on the corresponding cDNA from at least one tumor sample. Preferably, the method further comprises performing consensus sequencing on RNA or the corresponding cDNA from at least one tumor sample, preferably wherein the RNA is poly-(A) selected mRNA and/or 5′ cap containing mRNA. Preferably, the method further comprises selecting poly-(A) mRNA from said tumor sample and performing long-read RNA sequencing or long-read cDNA sequencing based on the poly-(A) selected mRNA.

In one aspect, the disclosure provides a method for identifying neoantigen sequences, said method comprising:
performing whole genome sequencing of a tumor sample and a healthy sample from an individual,
   optionally performing long-read whole genome sequencing of a tumor sample and a healthy sample from the individual,
performing long-read RNA sequencing on RNA or long-read sequencing on the corresponding cDNA from at least one tumor sample, preferably wherein the RNA is poly-(A) selected mRNA and/or 5′ cap containing mRNA;
   optionally performing short-read RNA sequencing on RNA or short-read sequencing on the corresponding cDNA from at least one tumor sample,
   optionally performing consensus sequencing on RNA or the corresponding cDNA from at least one tumor sample, preferably wherein the RNA is poly-(A) selected mRNA and/or 5′ cap containing mRNA
identifying somatic genomic changes in nucleic acid sequences from at least one tumor sample from an individual, said step comprising:
determining the presence of cis-splicing mutations, wherein the mutation results in a tumor specific open reading frame,
determining the presence of intragenic frameshift mutations in polypeptide encoding sequences, wherein the mutation results in a tumor specific open reading frame,
determining the presence of DNA rearrangements resulting in new junctions of DNA sequences, wherein the DNA rearrangement results in a tumor specific open reading frame,
determining the predicted amino acid sequences encoded by the tumor specific open reading frames,
selecting, as neoantigen peptide sequences, amino acid sequences comprising at least 9 amino acids, wherein the neoantigen peptide sequences comprise at least four contiguous amino acids encoded by the tumor specific open reading frames.

In some embodiments, the method further comprises determining the presence of a mutation in a stop codon, wherein the mutation results in a tumor specific open reading frame. In some embodiments, the somatic genomic changes are selected from single nucleotide variants (SNVs), indels, and structural variants.

In one aspect, the disclosure provides a method for identifying neoantigen sequences, said method comprising:
performing whole genome sequencing of a tumor sample and a healthy sample from an individual,
   optionally performing long-read whole genome sequencing of a tumor sample and a healthy sample from the individual,
performing long-read RNA sequencing on RNA or long-read sequencing on the corresponding cDNA from at least one tumor sample, preferably wherein the RNA is poly-(A) selected mRNA and/or 5′ cap containing mRNA;
   optionally performing short-read RNA sequencing on RNA or short-read sequencing on the corresponding cDNA from at least one tumor sample,
   optionally performing consensus sequencing on RNA or the corresponding cDNA from at least one tumor sample, preferably wherein the RNA is poly-(A) selected mRNA and/or 5′ cap containing mRNA
identifying somatic genomic changes in nucleic acid sequences from at least one tumor sample from an individual, said step comprising determining the presence of single nucleotide variants (SNVs), indels, and structural variants that result in tumor specific open reading frames,
determining the predicted amino acid sequences encoded by the tumor specific open reading frames,
selecting, as neoantigen peptide sequences, amino acid sequences comprising at least 9 amino acids, wherein the neoantigen peptide sequences comprise at least four contiguous amino acids encoded by the tumor specific open reading frames.

Preferably, said method detects the presence of a) cis-splicing mutations, wherein the mutation results in a tumor specific open reading frame, b) intragenic frameshift mutations in polypeptide encoding sequences, wherein the mutation results in a tumor specific open reading frame, and c) DNA rearrangements resulting in new junctions of DNA sequences, wherein the DNA rearrangement results in a tumor specific open reading frame. Preferably, the method further comprises determining the presence of a mutation in a stop codon, wherein the mutation results in a tumor specific open reading frame.

In some embodiments of the methods disclosed herein, the DNA rearrangements resulting in new junctions of DNA sequences result in
the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence or
the fusion of at least part of the coding strand of a first gene to intergenic non-coding DNA or to the noncoding strand of a second gene.

In some embodiments, the RNA sequencing is performed using long-read direct RNA sequencing, preferably Nanopore sequencing, or long-read cDNA sequencing.

In some embodiments, the method further comprises selecting poly-(A) mRNA from said tumor sample and performing long-read RNA sequencing or long-read cDNA sequencing based on the poly-(A) selected mRNA.

In some embodiments, the method comprises mapping the genomic sequences obtained to a human reference sequence to identify somatic genomic changes in the tumor sample, wherein the somatic genomic changes result in new open reading frames.

In some embodiments, the method comprises generating an in silico reconstructed tumor-specific reference genome. In a particular embodiment, the method comprises:
a) performing whole genome sequencing of a tumor sample and a healthy sample from the individual,
   optionally performing long-read whole genome sequencing of a tumor sample and a healthy sample from the individual,
b) performing long-read RNA sequencing on RNA or long-read sequencing on the corresponding cDNA from at least one tumor sample to obtain RNA sequencing reads, preferably wherein the RNA is poly-(A) selected mRNA and/or 5′ cap containing mRNA;
c) optionally performing short-read RNA sequencing on RNA or short-read sequencing on the corresponding cDNA from at least one tumor sample, preferably wherein the RNA is poly-(A) selected mRNA and/or 5′ cap containing mRNA;
d) mapping the genomic sequences obtained from the tumor tissue and corresponding healthy tissue to a human reference sequence to identify DNA rearrangements in the tumor sample,
e) generating in silico a reconstructed tumor-specific reference genome comprising the identified somatic DNA rearrangements;
f) aligning the RNA sequencing reads to the reconstructed tumor-specific reference genome;
g) determining the sequences of the full-length RNA transcripts encoded by nucleic acid sequences comprising the somatic DNA rearrangements;
h) determining the amino acid sequences encoded by the full-length transcripts of g),
i) selecting, as candidate neoantigen sequences, sequences comprising at least 9 contiguous amino acids of the amino acid sequence of h), wherein at least four of the contiguous amino acids are not encoded in the germline genome of the individual.

In a particular embodiment, the method comprises:
a) performing whole genome sequencing of a tumor sample and a healthy sample from the individual,
   optionally performing long-read whole genome sequencing of a tumor sample and a healthy sample from the individual,
b) performing long-read RNA sequencing on RNA or long-read sequencing on the corresponding cDNA from at least one tumor sample to obtain RNA sequencing reads, preferably wherein RNA is poly-(A) selected mRNA and/or 5′ cap containing mRNA;
c) optionally performing short-read RNA sequencing on RNA or short-read sequencing on the corresponding cDNA from at least one tumor sample, preferably wherein RNA is poly-(A) selected mRNA and/or 5′ cap containing mRNA;
d) aligning the RNA sequencing reads to a human reference sequence;
e) mapping the genomic sequences obtained from the tumor tissue and corresponding healthy tissue to a human reference sequence to identify DNA rearrangements in the tumor sample,
f) identification of a linear contig of DNA sequence from the tumor genomic sequences that comprises a DNA rearrangement and comprises genomic segments that align to RNA sequencing reads;
g) generating in silico a reconstructed tumor-specific reference genome comprising the identified DNA rearrangement to which the RNA sequencing reads align;
h) aligning the RNA sequencing reads to the reconstructed tumor-specific reference genome;
i) determining the sequences of the full-length RNA transcripts encoded by nucleic acid sequences comprising the somatic DNA rearrangements;
j) determining the amino acid sequences encoded by the full-length transcripts of i),
k) selecting, as candidate neoantigen sequences, sequences comprising at least 9 contiguous amino acids of the amino acid sequence of j), wherein at least four of the contiguous amino acids are not encoded in the germline genome of the individual.

The disclosure also provides a method for preparing a vaccine or collection of vaccines for the treatment of cancer in an individual, comprising identifying candidate neoantigen peptide sequences according to any of the preceding embodiments and preparing a vaccine or collection of vaccines comprising peptides having said amino acid sequences or comprising nucleic acids encoding said amino acid sequences.

Preferably, the candidate neoantigen peptide sequences comprise amino acid sequences encoded by cis-splicing mutations as defined above.

Preferably, the candidate neoantigen peptide sequences comprise amino acid sequences encoded by nucleic acid sequences comprising a mutation in a stop codon as defined above.

Preferably, the candidate neoantigen peptide sequences comprise amino acid sequences encoded by:
nucleic acid sequences comprising intragenic frameshift mutations as defined above,
nucleic acid sequences comprising DNA rearrangements that form new junctions of DNA sequences, wherein the DNA rearrangement results in the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence, and/or
nucleic acid sequences comprising DNA rearrangements that form new junctions of DNA sequences, wherein the DNA rearrangement results in the fusion at least part of the coding strand of a first gene to intergenic non-coding DNA or to the noncoding strand of a second gene (i.e., Hidden Frames). In preferred embodiments, the vaccine comprises Hidden Frame neoantigens.

Preferably, said method for preparing a vaccine or collection of vaccines comprises:
i) selecting from the candidate neoantigen peptide sequences identified, neoantigen peptide sequences having one or more of the following characteristics:
   neoantigen peptide sequences which do not share a contiguous stretch of at least 4 amino acids with human protein reference sequences;
   neoantigen peptide sequences wherein the genomic variant allele frequency of the respective somatic mutation in the tumor cells of a tumor sample is at least 0.1;
   neoantigen peptide sequences wherein the cysteine content for each peptide is 30% or less, where cysteine content (Qcys) is defined as the number of cysteines in said sequence divided by the total number of amino acids in said sequence;
   neoantigen peptide sequences for which the underlying somatic mutations have a maximum distance with regard to chromosomal location, preferably wherein each mutation is located on a different chromosomal arm; and
   neoantigen peptide sequences wherein the peptides are predicted to comprise one or more MHC I and/or MHC II binding epitope; and
ii) preparing a vaccine or collection of vaccines comprising peptides having the selected neoantigen amino acid sequences or nucleic acids encoding the selected amino acid sequences.

Preferably, said vaccine or collection of vaccines comprises essentially all candidate neoantigen peptides identified, or nucleic acids encoding said peptides.

Preferably, the vaccine or collection of vaccines comprises at least 100 amino acids corresponding to the candidate neoantigen peptide sequences encoded by the new open reading frames.

Preferably, the vaccine or collection of vaccines comprises at least 300 or 400, preferably at least 1000, amino acids corresponding to the candidate neoantigen peptide sequences encoded by the new open reading frames.

Preferably, the cancer is not micro-satellite instable (MSI).

In a preferred embodiment, the invention provides a vaccine or collection of vaccines for the treatment of cancer, obtainable by a method as disclosed herein.

In a preferred embodiment, the invention provides a vaccine or collection of vaccines for use in the treatment of cancer in an individual. Methods are also described for treating cancer comprising administering to an individual in need thereof a vaccine or collection of vaccines as disclosed herein and/or as obtainable by a method as disclosed herein.

The invention further provides a vaccine or collection of vaccines for the treatment of cancer wherein the vaccine comprises a neoantigen peptide, or nucleic acid encoding said neoantigen peptide. Preferably, the vaccine or collection of vaccines are obtainable by a method as disclosed herein. In some embodiments, the vaccine comprises at least two different neoantigen peptides. In some embodiments, the at least two different neoantigen peptides are linked, preferably wherein said peptides are comprised within the same polypeptide.

The invention further provides methods of treating an individual in need thereof with said vaccines. In particular, methods for the treatment of cancer are provided comprising administering to an individual in need thereof a vaccine or collection of vaccines as disclosed herein.

In a preferred embodiment the neoantigen peptide or collection of neoantigen peptides can serve as a bait to select or to identify T-cells isolated from a cancer patient, or to stimulate said T-cells. In one aspect the disclosure provides a method for preparing a cellular immunotherapy for the treatment of cancer in an individual, said method comprising contacting T-cells with the candidate neoantigen peptide sequences identified from the individual according to any one of the methods described herein. Preferably, the neoantigen peptide is bound to an MHC-I molecule. In some embodiments, the T-cells are obtained from said individual. In some embodiments, contacting T-cells with the candidate neoantigen peptide sequences results in the stimulation of the T-cells. In some embodiments, the method comprises selecting T-cells having specificity for one or more of said neoantigen peptide sequences. In some embodiments, the method further comprises the in vitro expansion of the stimulated and/or selected T-cells. In some embodiments, the methods may further comprise the isolation of a T-cell receptor or a collection of T-cell receptors with specificity for one or more of said neoantigen peptide sequences.

The disclosure also provides the following preferred embodiments.
1. A method for identifying neoantigen sequences, said method comprising
i) performing whole genome sequencing of at least one tumor sample and at least one healthy sample from an individual,
ii) performing long-read RNA sequencing on RNA or long-read sequencing on the corresponding cDNA from the at least one tumor sample;
iii) identifying somatic genomic changes in nucleic acid sequences from at least one tumor sample from an individual, said step comprising determining the presence of single nucleotide variants (SNVs), indels, and structural variants that result in tumor specific open reading frames,
   wherein said step comprises:
      determining the presence of cis-splicing mutations that result in tumor specific open reading frames;
      determining the presence of intragenic frameshift mutations in polypeptide encoding sequences, wherein the mutation results in a tumor specific open reading frame,
      determining the presence of DNA rearrangements resulting in new junctions of DNA sequences, wherein the DNA rearrangement results in a tumor specific open reading frame, and
      determining the presence of a mutation in a stop codon, wherein the mutation results in a tumor specific open reading frame;
iv) determining the predicted amino acid sequences encoded by the tumor specific open reading frames, and
v) selecting, as candidate neoantigen peptide sequences, amino acid sequences comprising at least 8, preferably at least 9, amino acids, wherein the neoantigen peptide sequences comprise at least one amino acid, preferably at least 4 contiguous amino acids, encoded by a tumor specific open reading frame.
2. The method of embodiment 1, wherein step i) comprises performing long-read whole genome sequencing of the at least one tumor sample and at least one healthy sample from the individual.
3. The method of any one of the preceding embodiments, comprising performing long-read RNA sequencing on RNA or long-read sequencing on the corresponding cDNA from at least one tumor sample, wherein the RNA is poly-(A) selected mRNA and/or 5′ cap containing mRNA, preferably wherein the poly-(A) and/or 5′ cap containing mRNA is selected by a purification step.
4. The method of any one of the preceding embodiments, wherein the RNA sequencing is performed using long-read direct RNA sequencing, preferably Nanopore sequencing, or long-read cDNA sequencing.
5. The method of any one of the preceding embodiments, further comprising performing short-read RNA sequencing on RNA or short-read sequencing on the corresponding cDNA from at least one tumor sample.
6. The method of any one of the preceding embodiments, further comprising performing consensus sequencing on RNA or the corresponding cDNA from at least one tumor sample, preferably wherein the RNA is poly-(A) selected mRNA and/or 5′ cap containing mRNA.
7. The method of any one of the preceding embodiments, wherein the method further comprises selecting poly-(A) mRNA from said tumor sample and performing long-read RNA sequencing or long-read cDNA sequencing based on the poly-(A) selected mRNA.
8. The method of embodiment 7, wherein the method further comprises selecting 5′ cap containing mRNA from said tumor sample and performing long-read RNA sequencing or long-read cDNA sequencing based on the selected mRNA.
9. The method of any of the preceding embodiments, wherein the selected candidate neoantigen peptide sequences comprise amino acid sequences resulting from cis-splicing mutations that result in tumor specific open reading frames, preferably wherein the method further comprises comparing the splice junction resulting from the cis-splicing mutation with a database of mRNA wild-type splice junctions, and selecting as candidate neoantigen peptide sequences those sequences where said splice junction is not present in the database of mRNA wild-type splice junctions.
10. The method of any of the preceding embodiments, wherein the selected candidate neoantigen peptide sequences comprise amino acid sequences resulting from:
   intragenic frameshift mutations in polypeptide encoding sequences that result in tumor specific open reading frames;
   DNA rearrangements resulting in new junctions of DNA sequences, wherein the DNA rearrangement results in a tumor specific open reading frame; and/or
   mutation in a stop codon, wherein the mutation results in a tumor specific open reading frame.
11. The method of any of the preceding embodiments, wherein said method comprises defining tumor specific open reading frames by determining strings of one or more consecutive tumor specific amino acids, where an amino acid is considered tumor specific if
(i) the position of the first nucleotide of the triplet encoding the amino acid does not align to a genomic position which is a known wild-type P-site;
(ii) the amino acid is part of at least one k-mer amino acid sequence which does not correspond to a known wild-type human peptide, wherein k is at least 8, preferably 8, 9, 10, or 11; and
(iii) the amino acid is encoded by a genomic sequence that is downstream of the somatic genomic change, wherein for a cis-splicing mutation each amino acid of said string of one or more consecutive novel amino acids is encoded by a genomic sequence that is downstream of the first novel splice junction.
12. The method of any of the preceding embodiments, wherein the method comprises selecting neoantigen peptide sequences having one or more of the following characteristics:
   neoantigen peptide sequences which do not share a contiguous stretch of at least 4 amino acids with human protein reference sequences;
   neoantigen peptide sequences wherein the genomic variant allele frequency of the respective somatic mutation in the tumor cells of a tumor sample is at least 0.1;
   neoantigen peptide sequences wherein the cysteine content for each peptide is 30% or less, where cysteine content (Qcys) is defined as the number of cysteines in said sequence divided by the total number of amino acids in said sequence;
   neoantigen peptide sequences for which the underlying somatic mutations have a maximum distance with regard to chromosomal location, preferably wherein each mutation is separated by at least 20 Mb, at least 50 Mb, or at least 100 Mb, more preferably wherein each mutation is located on a different chromosomal arm;
   neoantigen peptide sequences wherein the peptides are predicted to comprise one or more MHC I and/or MHC II binding epitopes; and
   neoantigen peptide sequences for which the RNA expression level of the underlying transcripts encoding such neoantigen peptide sequences have a gene expression value of at least 0.1 transcript per million (TPM) in the tumor sample.
13. The method of any of the preceding embodiments, comprising identifying candidate neoantigen sequences from a plurality of individuals and selecting as shared candidate neoantigen sequences, candidate neoantigen peptide sequences identified from at least two individuals.
14. A method for preparing a vaccine or collection of vaccines for the treatment of cancer in an individual, comprising identifying and selecting candidate neoantigen peptide sequences according to any of the preceding embodiments and preparing a vaccine or collection of vaccines comprising one or more peptides having said amino acid sequences or comprising one or more nucleic acid molecules encoding said amino acid sequences.
15. A method for preparing an antigen or a collection of antigens comprising identifying and selecting candidate neoantigen peptide amino acid sequences according to any of embodiments 1-13 and preparing an antigen or collection of antigens comprising one or more peptides having said amino acid sequences or comprising one or more nucleic acid molecules encoding said amino acid sequences.
16. The method of any one of embodiments 14-15, wherein said amino acid sequences encoded by the tumor specific open reading frames comprise at least 50 amino acids.
17. The method of any one of embodiments 14-16, wherein said vaccine, collection of vaccines, antigen, or collection of antigens, respectively, comprise or encode essentially all candidate neoantigen peptides identified.
18. The method of any one of embodiments 14-17, wherein said nucleic acid molecule or collection of nucleic acid molecules comprises deoxyribonucleic acid (DNA) and/or ribonucleic acid (RNA).
19. The method of embodiment 18, wherein said nucleic acid molecule is mRNA, self-amplifying RNA, circular RNA, or viral RNA, preferably mRNA.
20. The method of embodiments 18 or 19, additionally comprising a step of RNA in vitro transcription.
21. The method of embodiments 18 to 20, additionally comprising a step formulating the nucleic acid molecule or collection of nucleic acid molecules, preferably the RNA, in a lipid-based carrier, preferably wherein said lipid-based carrier is selected from lipid nanoparticles, liposomes, lipoplexes, and nanoliposomes.
22. A vaccine or collection of vaccines for the treatment of cancer, obtainable by a method according to any one of embodiments 14, or 16-21.
23. A peptide antigen or collection of peptide antigens obtainable by the method according to any one of embodiments 15-17.
24. An isolated nucleic acid molecule or collection of nucleic acid molecules that encode the peptide antigen or collection of peptide antigens of embodiment 23, preferably wherein the nucleic acid molecule or collection of nucleic acid molecules comprises deoxyribonucleic acid (DNA) and/or ribonucleic acid (RNA).
25. A peptide antigen obtainable by identifying candidate neoantigen peptide amino acid sequences according to any one of embodiments 1-13 and preparing a peptide comprising one or more of said neoantigen peptide amino acid sequences.
26. An isolated nucleic acid molecule encoding the peptide antigen of embodiment 25, preferably wherein the nucleic acid molecule or collection of nucleic acid molecules comprises deoxyribonucleic acid (DNA) and/or ribonucleic acid (RNA).
27. A pharmaceutical composition comprising
i) the nucleic acid molecule or collection of nucleic acid molecules from any one of embodiments 24 or 26, the one or more nucleic acid molecules obtainable by a method of any one of embodiments 14-20, and the vaccine or collection of vaccines obtainable by a method according to any one of embodiments 14-21, and the vaccine or collection of vaccines according to embodiment 22; and comprising one or more nucleic acid molecules and
ii) a lipid-based carrier, preferably wherein said lipid-based carrier is selected from lipid nanoparticles, liposomes, lipoplexes, and nanoliposomes.
28. A binding molecule or collection of binding molecules that binds the peptide antigen according to embodiment 23 or 25 or the collection of peptide antigens according to embodiment 23, wherein the binding molecule is an antibody, a T-cell receptor, or an antigen binding fragment thereof.
29. A chimeric antigen receptor or collection of chimeric antigen receptors that binds the peptide antigen according to embodiment 23 or 25 or the collection of peptide antigens according to embodiment 23, wherein each chimeric antigen receptor comprises i) a T cell activation molecule; ii) a transmembrane region; and iii) an antigen recognition moiety.
30. One or more T-cells expressing the T-cell receptor or collection of T-cell receptors of embodiment 28 or the chimeric antigen receptor or collection of chimeric antigen receptors of embodiment 29.
31. The vaccine or collection of vaccines according to embodiment 22, the peptide antigen or collection of peptide antigens according to embodiment 23 or 25, the nucleic acid molecule or collection of nucleic acid molecules according to embodiment 24 or 26, the pharmaceutical composition of embodiment 27, the binding molecule or collection of binding molecules of embodiment 28, the T-cell receptor or collection of T-cell receptors of embodiment 28, the chimeric antigen receptor or collection of chimeric antigen receptors of embodiment 29, or the one or more T-cells of embodiment 30, for use in the treatment of cancer, preferably cancer in an individual.
32. A method for preparing a cellular immunotherapy for the treatment of cancer, said method comprising contacting T-cells with one or more candidate neoantigen peptide sequences identified from the individual according to any one of embodiments 1-13 to produce a cellular immunotherapy.
33. The method according to embodiment 32, further comprising selecting T-cells with specificity for one or more of said neoantigen peptide sequences.
34. The method according to embodiment 32 or 33, wherein said contacting results in the stimulation of the T-cells.
35. The method according to any one of embodiments 32-34, further comprising the in vitro expansion of stimulated and/or selected T-cells.
36. The method according to any one of embodiments 32-35, wherein the T-cells are obtained from said individual.
37. The method according to any one of embodiments 32-36, further comprising the identification of or sequencing of a T-cell receptor or a collection of T-cell receptors with specificity for one or more of said neoantigen peptide sequences.
38. The method according to any one of embodiments 32-37, wherein said contacting step comprises contacting T-cells with antigen-presenting cells transfected with one or more candidate neoantigen peptides or one or more nucleic acid molecules encoding the one or more candidate neoantigen peptides.
39. The method of embodiment 38, comprising transfecting T-cells with one or more nucleic acid molecules that encode for a T-cell receptor with specificity for one or more of said neoantigen peptide sequences.
40. A cellular immunotherapy for use in the treatment of cancer, preferably cancer in an individual, wherein said cellular immunotherapy comprises the administration of T-cells prepared according to a method of any one of embodiments 32-39.
41. A method of treating cancer, preferably cancer in an individual, the method comprising
i) performing whole genome sequencing of a tumor sample and a healthy sample from an individual in need thereof,
ii) performing long-read RNA sequencing on RNA or long-read sequencing on the corresponding cDNA from at least one tumor sample;
iii) identifying somatic genomic changes in nucleic acid sequences from at least one tumor sample from an individual, said step comprising determining the presence of single nucleotide variants (SNVs), indels, and structural variants that result in tumor specific open reading frames,
   wherein said step comprises:
      determining the presence of cis-splicing mutations that result in tumor specific open reading frames;
      determining the presence of intragenic frameshift mutations in polypeptide encoding sequences, wherein the mutation results in a tumor specific open reading frame,
      determining the presence of DNA rearrangements resulting in new junctions of DNA sequences, wherein the DNA rearrangement results in a tumor specific open reading frame, and
      determining the presence of a mutation in a stop codon, wherein the mutation results in a tumor specific open reading frame;
iv) determining the predicted amino acid sequences encoded by the tumor specific open reading frames,
v) selecting, as candidate neoantigen peptide sequences, amino acid sequences comprising at least 8 amino acids, wherein the neoantigen peptide sequences comprise at least one amino acid encoded by a tumor specific open reading frame, and
vi) administering to said individual
   a peptide antigen or a collection of peptide antigens comprising at least one of said candidate neoantigen peptide sequences,
   one or more nucleic acid molecules encoding at least one of said candidate neoantigen peptide sequences,
   one or more T-cells expressing T-cell receptors or chimeric antigen receptors with specificity for at least one of said candidate neoantigen peptide sequences.

### BRIEF DESCRIPTION OF THE DRAWINGS

FIG. 1: Outline of a point mutation leading to a single amino acid change in a protein (missense mutation). A single amino acid change provides only limited possibility for immune recognition.

FIG. 2: Outline of a short insertion (A) mutation leading to a frameshift and a novel Frame peptide sequence that forms a long foreign sequence and is an optimal substrate for immune recognition.

FIG. 3: Outline of a structural genomic variation leading to a novel expressed sequence derived from non-coding DNA. The novel expressed sequence is spliced to the 5′ exons of a known gene and results in a novel long peptide sequence, denoted as a Hidden Frame. Of note, out-of-frame fusion genes may also originate from structural variation in the tumor genome, but would involve the fusion of a 5′ donor gene and a 3′ acceptor gene, instead of a non-coding genomic region.

FIG. 4: High-level overview of Splice-Frame detection procedure as outlined herein. Of note, this figure focuses on the methodology to process long and short RNA reads. Methodology for detection of somatic mutations in the cancer genome are not depicted as this is done using state-of-the-art whole genome sequencing approaches.

FIG. 5: Outline of the effects of a Gain of Splice (GOS) mutation on transcript splicing. A tumor-specific genetic mutation is depicted that introduces a novel splice site. This leads to novel transcript sequences that deviate from the known transcript structure. In the depicted example, two novel RNA splice junctions are indicated. Both novel splice junctions are considered to predict a novel open reading frame encoded by the transcripts containing these novel splice junctions. This leads to the expression of a splice Frame-encoding transcript.

FIG. 6: Outline of the effects of a Loss of Splice (LOS) mutation on transcript splicing. A tumor-specific genetic mutation is depicted that affects a known splice site. This may lead to either an extension of the 5′ or 3′ end of a known exon. Alternatively (lower panel), a retained intron may emerge from LOS mutation. In both cases, this results in a splice Frame neoantigen.

FIG. 7: Outline of the effects of a genomic structural variant on novel transcript splicing. In the depicted example a tandem duplication present in the genome of a patient's tumor leads to the duplication of an exon of a known gene. Sequencing of entire transcript molecules of the tumor identified mRNAs that contain the duplicated exon. In silico translation of the mRNA molecules subsequently reveals a splice Frame.

FIG. 8: For detection of a Loss of Function (LOS) splice mutation, novel RNA junctions are identified in a pre-defined ‘effect zone’. This effect zone is typically extending from the intron before the splice mutation to the intron after the splice mutation, but alternative effect zones may be used. Novel RNA splice junctions observed in the effect zone are considered as a starting point to detect splice Frames. Those novel RNA junctions are typically compared to databases of existing junctions (e.g. GTEx, or in house databases). Furthermore, the novel RNA junctions are preferably observed in both short-read and long-read RNA sequencing data of the same tumor sample.

FIG. 9: Example of a splice donor mutation leading to a retained intron in the LIG1 gene in a non-small cell lung tumor. The genetic mutation involves a C->A point mutation, which is observed in all intronic reads from the short-read and long-read RNA sequencing of this tumor.

FIG. 10: Schematic overview of local genome reconstruction informed by somatic structural genomic rearrangement breakpoint junctions. A segment from the normal human reference genome (e.g. GRCh37 or GRCh38 or the like) has been deleted in a specific tumor. The genome reconstruction involves the generation of a contig that lacks the deleted segment. This is a simplified example and in practice much more complex rearrangements occur with neighboring breakpoint junctions leading to complex local genome configurations.

FIG. 11: Example of intragenic tandem duplication in the KLF5 gene in a tumor genome. Long Nanopore (cDNA) transcript reads were mapped to a reconstructed contig containing the tandemly duplicated sequence. The novel transcript sequence discovered by the Nanopore reads involves tandemly duplicated exons which encode a novel (Splicing) Frame sequence. The tandemly duplicated exonic structure could only be resolved by aligning the long-read Nanopore cDNA reads to a tumor-specific genomic contig containing the tandemly duplicated segments.

FIG. 12: Schematic drawing outlining correction of erroneous splice junctions in long transcript reads based on splice junctions observed in short RNA reads. Long RNA reads derived from single molecule sequencing are inherently erroneous. Hence, correction of the splice junctions identified by the long RNA reads must be performed. A preferred way to do this is by correcting the long read RNA splice junction based on highly accurate short-read RNA splice junctions. By such correction process, long transcript reads can be obtained which contain information on overall transcript structure (e.g. which exons are included in the transcript) and on the exact splice junctions contained within the transcript. Alternative methods for obtaining accurate long read RNA sequences exists, e.g. circular consensus sequencing, as described herein.

FIG. 13: Schematic drawing of splice correction procedure involving an erroneous long read and two potential correct splice junctions (1, 2) in two groups of short reads. Here R1 and R2 represent the number of short reads spanning each junction. F₁/F₂ and T₁/T₂ are the 5′ and 3′ distances of the long-read splice sites with the 5′ and 3′ splice sites of junction 1 and 2. N_{F}_{₁}_{T}_{₁} and N_{F}_{₂}_{T}_{₂} are the number of times other long read junctions in the same sample/mapping file had a single short read junction nearby with an offset of F₁T₁ and F₂T₂, respectively. The probabilities P₁ and P₂ are calculated as indicated. The short-read splice junction with the highest probability is chosen. A minimum probability cutoff can be set to consider the junction confidently corrected.

FIG. 14: Example of the correction of long RNA reads with short RNA reads. Two short read splice junctions are indicated (SJ_{A}, SJ_{B}). These splice junctions are used to correct long read RNA sequences (middle panel) into corrected long read RNA sequences (bottom panel). Two groups of corrected long RNA read are depicted one of which is corrected by splice junction SJ_{A} and the other is corrected by splice junction SJ_{B}.

FIG. 15: Example of a splice acceptor mutation in a lung tumor leading to an alternative (downstream) splice acceptor site in the TP53 gene. This novel splice junction is tumor-specific (i.e. not observed in control short RNA reads) and leads to a shift in the reading frame of TP53 thereby resulting in an expressed novel splice Frame neoantigen.

FIG. 16: Example of a splice donor mutation in a lung tumor leading to retention of an intron in the LIG1 gene. The retained intron encodes a tumor-specific splice Frame.

FIG. 17: Example of a gain of splice point mutation in a lung tumor leading to a novel exon in the TPD52L1 gene. The novel exon is observed in both short and long read RNA sequencing data from the tumor and encodes a tumor-specific splice Frame.

FIG. 18: Example of a gain of splice point mutation in a lung tumor leading to a novel exon in the CCDC91 gene. The novel exon is observed in both short and long read RNA sequencing data from the tumor and encodes a tumor-specific splice Frame. A novel splice junction is observed in between this novel exon and the last coding exon of the CCDC91 gene.

FIG. 19: Schematic outline of two types of intra-genic deletions that may affect exon splicing patterns of a gene. The upper panel depicts an exonic deletion and the lower panel an intronic deletion. More complex situations may also occur, for example when a deletion covers part of an exon and part of an intron (i.e. crossing an exon-intron boundary). Novel transcript splice junctions are identified in the ‘effect zone’, which represents a search area covering and flanking the deletion interval. Identification of novel transcript splice junctions within the indicated effect zone is preferred over considering the entire gene body as the effect zone. Novel transcript splice junction that are more distant from the deletion interval (i.e. outside of the effect zone) are less likely to be caused by the deletion.

FIG. 20: Example of an intragenic deletion that results in a novel exon-exon junction in a lung tumor. The deletion covers a known exon of the IL7R gene. Short read and long read RNA transcript sequences are aligned to the human reference genome and both support the presence of transcripts that do not contain the deleted exon. Thus, a novel exon-exon junction is created that leads to a splice Frame neoantigen.

FIG. 21: Example of an intragenic deletion that results in a novel exon-exon junction of the OXCT1 gene in a lung tumor. The deletion covers four known exons of the OXCT1 gene. Short read and long read RNA transcript sequences are aligned to the human reference genome and both support the presence of transcripts that do not contain the deleted exons. Thus, a novel exon-exon junction is created that leads to a splice Frame neoantigen.

FIG. 22: Barplot showing Framome sizes of a series of tumor samples analyzed by the methodology described herein. Splice Frames are contributing to the total Framome of several of these tumors, indicating their importance for design of Frame-based immunotherapies.

FIG. 23: Framome plot depicting all expressed Frames of a single lung tumor. The Frames derived from splice mutations are indicated with an asterisk. A significant proportion of the total Framome is contributed by Splice Frames, indicating their importance for design of optimal Framome-based immunotherapies.

FIG. 24: Barplot indicating the number of novel Gain-of-splice junctions identified in short-read and long-read RNA sequencing data of a set of tumor specimens. These data show that most novel short-read RNA junctions are not observed in corresponding long-read RNA data.

FIG. 25: Example of mismapping of short RNA sequence reads in a repetitive intronic area of the genome. Long mRNA sequencing reads are aligned as expected only to exonic sequences with little noisy mappings in the intronic regions. Instead, multiple short RNA sequences are mapped erroneously mapped to a repetitive genomic region in an intron of the NEDD4L gene. This mismapping of short RNA reads leads to the detection of erroneous RNA splice junctions, which contributes to false positive discovery of splice Frames. Hence, a combined approach based on evidence in both short-read and long-read RNA sequencing data, provides more accurate detection of splice Frames.

FIG. 26: Schematic outline depicting the steps involved in the identification of splice Frames from long (corrected) transcript sequence reads. The long transcript sequences are each aligned to a (tumor-specific) reference genome. Subsequently, all aligned exonic segments of the reads are concatenated into a single sequence. The presumed translation start is determined for each transcript sequence, based on overlap with annotated translation start sites from the Ensembl database. Subsequently, in silico translation of the transcript sequence is performed to determine the novel splice Frame neoantigenic sequence.

FIG. 27: Example of the complete exonic structure of individual transcript molecules for the TPD52L1 gene as determined based on a combination of long-read and short-read RNA sequencing of a tumor sample. The long transcript sequences are divided in sequences representing normal (known) splice isoforms and novel transcript sequences containing a novel exon. Splice isoforms can be clearly distinguished and the quantity of each splice isoform can be determined from the sequencing data. In addition, the sequence of each transcript molecule can be determined as outline in FIG. 26.

FIG. 28: Example of a Hidden Frame resulting from a complex genomic rearrangement in the 3′ flanking region of the POLE4 gene. The Figure depicts two breakpoint junctions (vertical lines) downstream of the gene. The novel sequence downstream of the POLE4 gene results in a novel splicing pattern of this gene and concomitant expression of a novel Hidden Frame neoantigen. The complete exonic structure of individual transcript molecules are depicted in the top part of the figure. Each individual transcript molecule can be translated to determine the ultimate Frame sequence.

FIG. 29: Identification of stoploss mutations in 18 different tumor samples. For 9 out of the 18 stoploss mutations the mutated allele was found to be expressed in RNA sequencing data.

FIG. 30: Example of a Stop Loss Frame resulting from mutation of a stop codon in a lung tumor.

FIG. 31: Overview of somatic mutation statistics for tumor samples analyzed by WGS in this study. Top panel indicates tumor purity (percentage of tumor cells) estimated from whole genome sequencing data. The lower three panels depict somatic variant counts for structural variants (SVs), single-nucleotide variants (SNVs) and indels.

FIG. 32: Long-read transcript sequencing statistics. (A) Long transcript reads mapped to the GAPDH gene, derived from a lung cancer (LUN011). The plot displays partial transcript reads in red and full length transcript reads in green. Known GAPDH splice variants are depicted in the lower part of the plot. (B) Overview of long-read RNA sequencing statistics for tumor samples generated by Nanopore sequencing devices. The top panel shows the read length distribution in a boxplot where a red triangle denotes the N50. The middle and lower panels indicate the number of reads per sample, and the percentage of full-length reads, respectively.

FIG. 33: Overview of FramePro pipeline. Tumor specific variants are identified from tumor/normal WGS and used in combination with short and long read RNA sequencing to reconstruct the tumor genome. RNA is remapped to this tumor specific reference to produce translatable full-length isoforms and a database of WT peptide k-mers and P-sites are used to identify which portions of these predicted peptides are novel. These NOPs are extracted to produce the Framome.

FIG. 34: Examples of each NOP category identified by FramePro. Reconstructed tumor contigs are shown as thick purple/green lines. Annotation isoforms from ENSEMBL are shown below the contigs. Full-length isoforms created through correction/collapsing of long-reads are shown above the contigs. The known/predicted protein coding structure of each isoform is provided with green for 5′-UTRs, brown for WT coding, red for NOP, multi-colored for zoomed-in NOP amino acids, and blue for 3′-UTRs. Non-coding isoforms are shown in grey. (A) An 8 Mb inversion within chromosome 9 leads to a fusion gene between the CAMSAP1 and URM1 genes in the glioblastoma sample GBM002. Beginning translation at the CAMSAP1 start site gives an NOP partially overlapping the 5′-UTR of URM1. (B) A basepair deletion in an exon of the BRF2 gene in lung sample LUN013 leads to out-of-frame translation of a portion of two exons. The 49 amino acid NOP represents an elongation of translation of the indel-containing isoform. (C) A point mutation in the head and neck tumor HAN001 leads to a splicing signal in the intron of the MLLT10 gene. This splicing leads to a partial 3′ intron retention and drives translation of a 10 amino acid NOP. (D) A point mutation within the stop codon of the CHCHD6 gene in the head-and-neck sample HAN002 leads to a translation elongation and a 15 amino acid NOP.

FIG. 35: Hidden NOPs are a frequent result of genomic structural variants. (A) Schematic outline of the origin of hidden NOPs. A somatic genomic breakpoint junction involving the 5′-end of a protein coding gene is fused to a non-coding genomic region. Transcription is driven by the promoter of the 5′-gene and continues across the structural variant breakpoint. The resulting transcript is spliced leading to a novel open reading frame encoding a tumor-specific NOP. (B) Example of a hidden NOP identified in LUN004, involving the TIMM8B gene. (C) Example of RiboSeq fragments across a hidden NOP involving the BCAS4 gene in MCF7 cells. (D) Barplot indicating RiboSeq signal for three different open reading frame phases for hidden NOPs identified in MCF7, A375 and 7860 cancer cell lines. The RiboSeq measurements were obtained only from the novel sequence (3′ portion) of the mRNA transcript encoding the hidden NOP.

FIG. 36: Analysis of NOPs across cancer types. (A) Framome sizes, as measured in number of amino acids across 61 tumor samples included in this study. Different categories of NOPs are indicated. (B) and (C) Examples of the framomes of a lung tumor (LUN013) and glioblastoma (GBM005). Each horizontal bar represents the amino acid sequence of a single NOP expressed by the tumor. Different amino acids are depicted using different colors. The NOP sequences are sorted by length. (D) NOP expression plotted against NOP genomic variant allele frequency. Each dot represents one NOP. Dot size indicates NOP length in amino acids. Variant allele frequencies were adjusted for tumor purity. NOP expression is measured as transcripts per million (TPM). (E) Overview of the origin of Hidden NOPs and out-of-frame gene fusions categorized by SV type. Intergenic=SV breakpoint junctions that do not affect a gene on each side of the junction. Intragenic=SV breakpoint junctions where the breakpoints are located in the same gene. Gene-Intergenic=SV breakpoint junctions involving a 5′-end of a gene coupled to a non-coding intergenic genomic regions. Gene-Gene=SV breakpoint junctions involving a 5′-gene fused to a 3′-gene in the correct orientation to form a fusion transcript. Complex hidden NOPs/gene fusions indicate the presence of complex genomic rearrangements underlying the novel transcript. The third column indicates the presence of multiple NOP events.

FIG. 37: The cancer neoantigen landscape.

FIG. 38: Example of a hidden NOP resulting from a complex chromosomal rearrangement in tumor sample BRE004. A tumor-specific complex chromosomal rearrangement involving five genomic junctions (SV junctions) was reconstructed using FramePro. Known isoform structures of the 5′ part of gene PRKDC are depicted below the reconstructed chromosome. A novel exon is formed at a non-coding intergenic region and the exon encodes a tumor-specific hidden NOP.

FIG. 39: Example of a genomic rearrangement resulting in the expression of multiple hidden NOPs in tumor sample BRE007. Corrected long RNA-seq reads are aligned onto a tumor-specific genomic contig involving a somatic SV. The left genomic segment encodes the 5′ part of the UCK2 gene, driving expression of novel transcripts that extend onto the right intergenic genomic segment. Multiple novel (tumor-specific) exons are formed on this intergenic genomic segment, representing different splice isoforms, resulting in the expression four different hidden NOPs.

FIG. 40: In silico determined immunogenic properties of NOPs. (A) Number of predicted MHC class I binding epitopes. For each NOP MHC class I binding epitopes were predicted using NetMHCPan. Epitopes are shown for each of the three major classes of NOPs (indels frameshift NOPs, Hidden NOPs, out-of-frame fusion gene NOPs). (B) Potential immunogenic epitopes contained in theoretical vaccines based on each analyzed tumor's framome (green lines) or missense mutations (blue line) for a vaccine of a given size. C) Self similarity distributions for random (n=14653), NOP (n=14653), and missense epitopes (n=68342) with mean scores shown. Significance was determined by a t-test. D) Correlation between in silico predicted and actual in vitro binding of framome-derived epitopes to relevant alleles. The analysis of netMHCpan predicted HLA class I binding and in vitro binding to various, patient respective HLA alleles was performed for epitopes with EL score <0.5 and <2, with green part of the bar depicting the number/percentage.

FIG. 41: Framomes of three lung cancer samples. MHC class I binding epitopes were predicted for each of the NOPs expressed by lung cancers LUN24, LUN26, LUN029. Class I HLA for each of the tumors are indicated. NOP sequences are indicated as bars, with each amino acid represented by a color. Lines underneath NOP sequences represent predicted class I epitopes.

FIG. 42: HLA binding and in vivo immunogenicity of NOPs. A) NOP-derived epitopes from three patients with NSCLC bound to patient specific HLA-A or B alleles as determined by in vitro binding assays. Positive control for each allele with high affinity was used to set 100% binding affinity and binding affinity of each peptide was calculated as relative to the positive control. Epitopes with at least 40% binding affinity are depicted. B) and C) Analysis of PBMCs of patient LUN029 using combinatorial coding and immune phenotyping. The cells double positive for Framome-derived epitope-HLA tetramer complex are considered specific for the epitope. Phenotyping of epitope-tetramer complex double positive cells (black dots) was performed by staining with anti-CD45RA, anti-CD27, and anti-PD-1 antibodies to determine antigen experience status of the cells. D) Genomic origin of hidden NOPs identified in patient LUN029 for which CD8+ T-cells were identified in patient PBMCs.

FIG. 43: Gating strategy. A) Selection of live CD3+CD8+ T cells. B) Gating of tetramer+CD3+CD8+ T cells.

FIG. 44: RNA-guided tumor genome reconstruction. (A) The chimeric path (p) consists of three alignments p₁, p₂, and p₃, shown as blue squares (exons) with thin black lines (introns), aligned to three different chromosomes depicted as colored arrows. Moving from 5′ to 3′ along the read, the chimeric alignments have chimeric introns m1 and m2, with higher and lower anchor points shown at the start/end of the alignments. The translocation SVs affecting these chromosomes are shown as black lines with segments overlapping the chromosomes in the direction of their breakend orientation. The breakend loci are shown as b₁, b₂, b₃, and b₄. (B) The breakend and chimeric intron genomic loci are represented as nodes in a directed graph. To account for the breakend orientation and partner connectivity, two nodes are needed for each breaken loci. Brown source breakend and chimeric intron nodes colored by chromosome lead to blue sink breakend nodes. Sink breakend nodes lead strictly to source breakend nodes. A path through the breakend nodes connecting the higher and lower chimeric intron nodes can be found for each chimeric intron m₁ and m₂. (C) The reconstructed tumor contig arising from concatenation of the chimeric intron graph walks. The long RNA read can be realigned to the contig to produce a linear alignment connecting the previously chimeric segments.

FIG. 45: Long RNA splice correction, isoform identification, and translation prediction. (A) High-accuracy short reads are used to correct the splice junctions of error prone long reads. If a unique short read junction is in the vicinity of the long read junction, then an unambiguous correction can be made. However if two sets of short reads (R₁ and R₂) support distinct short read junctions each in the correction window of the long read junction, a choice between the short read junctions must be made based on the 5′ offsets (F₁ and F₂) paired with the 3′ offsets (T1 and T2) taking into account their prior probability as described in section Methods 4.5.3. (B) Splice corrected reads are collapsed into isoforms based on their set of splice junctions. If two reads share the same splice junctions and their start/end are within a window ϵ_{isoform} basepairs of each other, then they are considered to support the same isoform. The isoform transcription start/end are taken as the maximum extension points of the underlying long reads. (C) RNA isoforms are translated by matching the initial splice junctions to known protein coding transcript structures. This figure depicts the case where a single annotated transcript in the region shares the first two exons in common with the observed isoform. This overlap supports starting translation of the RNA isoform at the same start codon as the known transcript.

### DETAILED DESCRIPTION OF THE DISCLOSED EMBODIMENTS

As used herein, the term “open reading frame” or ORF refers to a nucleic acid sequence comprising or encoding a continuous stretch of codons. As used herein the term “neoORF” refers to a tumor-specific open reading frame (i.e., novel open reading frame) arising from a somatic genomic change (i.e., mutation) including point mutations; indels; and DNA rearrangements, in particular structural variants. Such neoORFs are not present in the germline and/or healthy cells of an individual. Peptides arising from such neoORFs are referred to herein as neoantigens or ‘Frames’. The methods described herein have been developed, at least in part, in order to maximize the number of neoantigen amino acids identified from the tumor of an individual. As used herein, the term ‘Framome’ refers to all, or essentially all, of the neoORFs that result from somatic genetic changes as described herein (e.g., frameshift mutations, genomic rearrangements, splicing mutations, mutation of stop codon) that can be identified in a tumor sample using whole genome sequencing.

As used herein the term “sequence” can refer to a peptide sequence, DNA sequence or RNA sequence. The term “sequence” will be understood by the skilled person to mean either or any of these and will be clear in the context provided. For example, when comparing sequences to identify a match, the comparison may be between DNA sequences, RNA sequences or peptide sequences, but also between DNA sequences and peptide sequences. In the latter case the skilled person is capable of first converting such DNA sequence or such peptide sequence into, respectively, a peptide sequence and a DNA sequence in order to make the comparison and to identify the match. As is clear to a skilled person, when sequences are obtained from the genome or exome, the DNA sequences are preferably converted to the predicted peptide sequences. In this way, neo open reading frame peptides are identified. The neoantigens can include a polypeptide sequence or a nucleotide sequence encoding said polypeptide sequence.

As used herein the term “sample” can include a single cell or multiple cells or fragments of cells or an aliquot of body fluid, taken from an individual, by means including venipuncture, excretion, ejaculation, massage, biopsy, needle aspirate, lavage sample, scraping, surgical incision, or intervention or other means known in the art. The nucleic acid for sequencing is preferably obtained by taking a sample from a tumor of the patient. The skilled person knowns how to obtain samples from a tumor of a patient and depending on the nature, for example location or size, of the tumor. Preferably the sample is obtained from the patient by biopsy or resection. The sample is obtained in such manner that it allows for sequencing of the genetic material obtained therein. The biological material from multiple samples may also be used and/or pooled. As used herein, a sample may also be referred to as a biological sample. The sample may be from a tumor (or comprise tumor cells or tumor DNA).

The sample may also be a healthy sample from healthy tissue, i.e., a non-tumorous sample.

The term ‘individual’ includes mammals, both humans and non-humans and includes but is not limited to humans, non-human primates, canines, felines, murines, bovines, equines, and porcines. Preferably, the mammal is a human.

### Genetic Mutations and Neoantigens in Cancer

Cancer emerges because of mutational processes that affect the genome of cancer cells (Alexandrov and Stratton, Curr Opin Genet Dev 2014 February; 24(100):52-60). Various types of genetic mutations arise from such mutational processes, such as point mutations, short insertions and deletions and large structural genomic rearrangements. Mutations in cancer genomes may alter the amino acid composition of proteins, thereby leading to formation of neoantigens, which can represent tumor-specific antigens that can be recognized by the immune system and form a target of immunotherapy (Annual Review of Immunology Volume 37, 2019 Schumacher, pp 173-200).

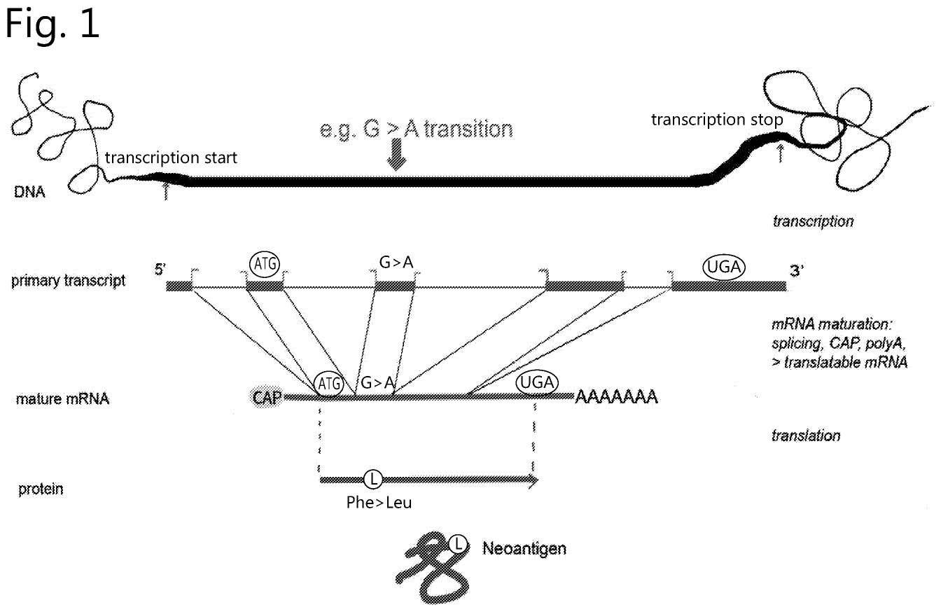

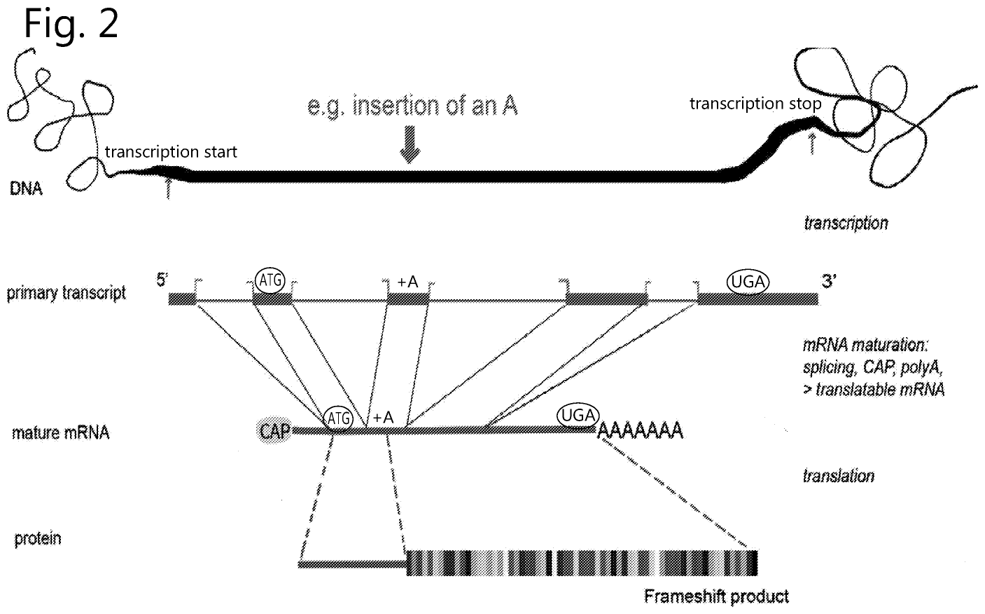

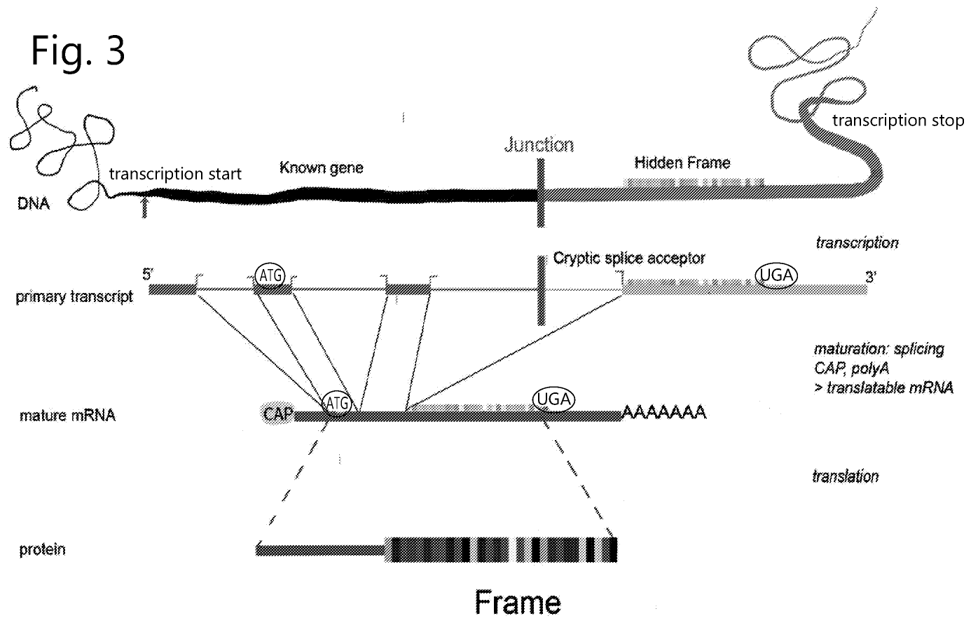

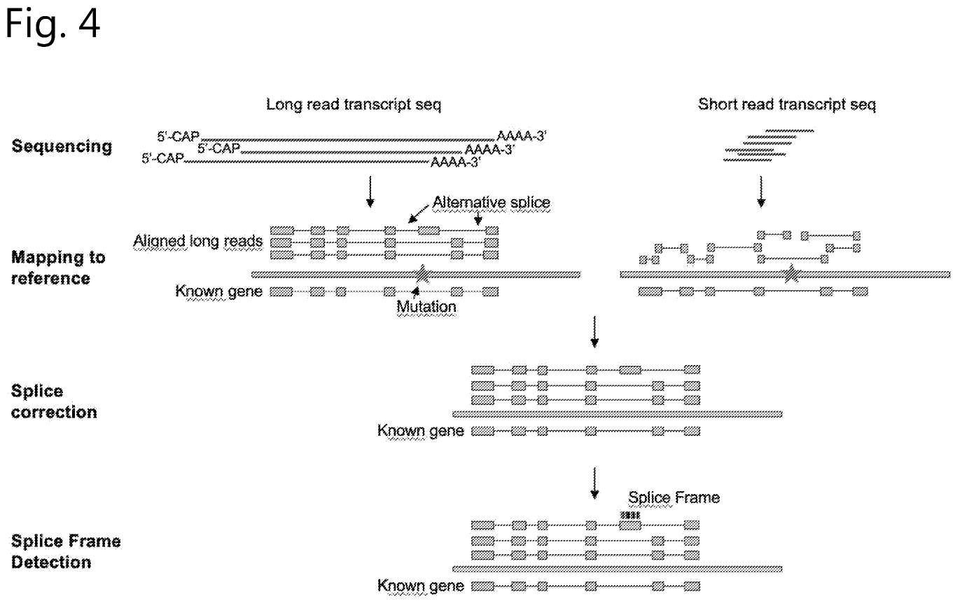

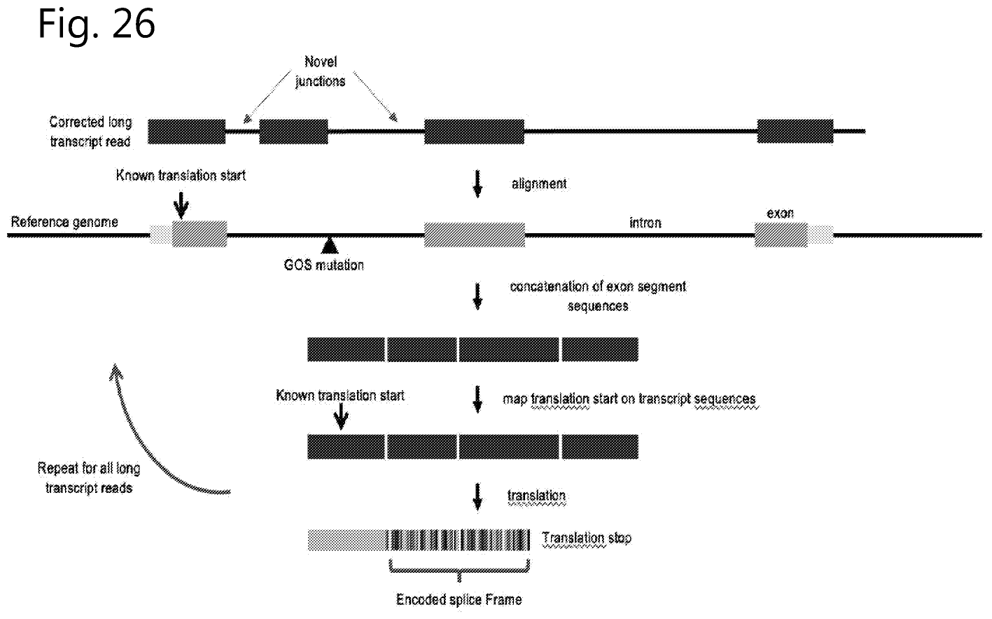

A typical neoantigen is formed by a non-synonymous point mutation in a coding exon, which changes one amino acid of a protein to another amino acid (FIG. 1; FIG. 26 Missense). If, instead, a frameshifting short insertion or deletion occurs in a coding sequence, a new stretch of amino acids may arise in a protein (FIG. 2; FIG. 26; Out-of-frame indel). The inventors previously described a new type of neoantigen that results from structural genomic rearrangements, whereby the 5′ part of a coding gene is fused to a non-coding genomic region, and whereby a novel chimeric mRNA molecule is produced that consists of part of the coding region of a known gene and one or more novel exons that are spliced out of the primary mRNA transcript (FIG. 3; FIG. 26 Hidden frame) (see also WO2021/172990).

The methods described herein identify neoantigen sequences. The use of neoantigen sequences for therapy has been described (e.g., WO2016/191545 and US2016/331822). However, the present methods determine the presence of single nucleotide variants (SNVs), indels, and structural variants that result in tumor specific open reading frames. In particular, the methods comprise determining the presence of cis-splicing mutations, determining the presence of intragenic frameshift mutations, determining the presence of DNA rearrangements and determining the presence of a mutation in a stop codon; wherein the mutations result in a tumor specific open reading frame. The methods combine whole genome sequencing and long-read RNA sequencing

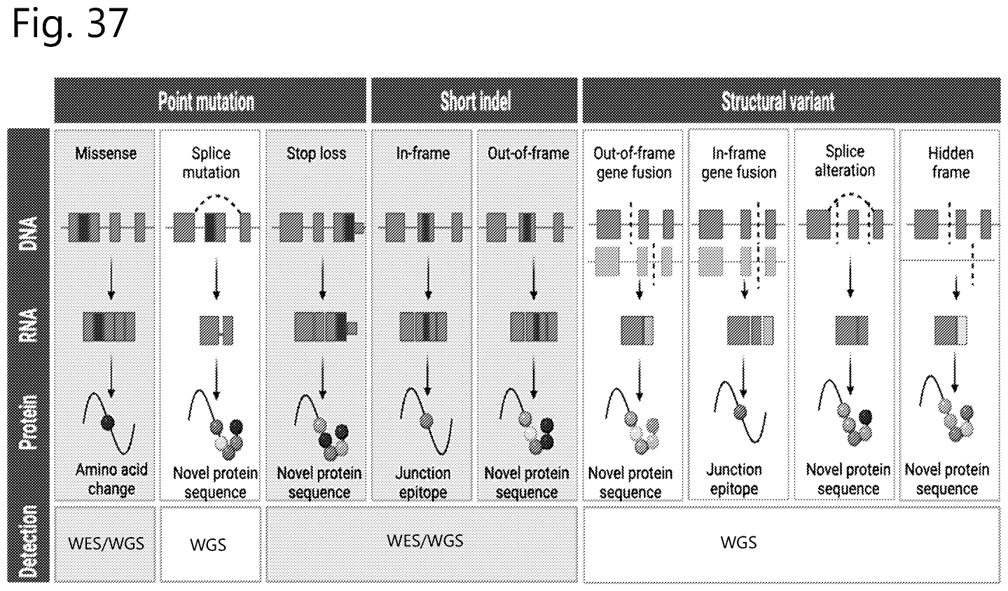

Neoantigens resulting from structural variants, such as frameshifts and “Hidden Frames” are known from WO2021172990 (see, e.g., FIG. 37). However, WO2021172990 fails to describe determining new Frame neoantigens due to SNVs. Tools such as pVAC (Hundal et al. (Cancer Immunology Research 2020) are known for calling SNVs. However, Hundal et al. fails to recognize the importance of identifying novel Frames that result from such SNVs nor does Hundal et al. recognize the importance of performing both whole genome sequencing and long-read RNA sequencing. As described further herein, given the complex and diverse patterns of transcript isoforms that are expressed in human tissues, an end-to-end sequence of the entire structure of a transcript is required to predict the translated sequences that may emerge from aberrant (mis-spliced) transcripts resulting from genetic mutations.

Neoantigens Resulting from Mis-Splicing of mRNA

Recent work has described yet another category of neoantigens, which are a consequence of genetic mutations that alter splice donor and acceptor sites, thereby giving rise to novel (alternatively spliced) transcripts. Mis-splicing mutations have been comprehensively described by Jung et al (Oncogene volume 40:1347-1361 (2021)), who used a combination of whole-genome sequencing (WGS) and short-read transcriptome sequencing (RNA-seq) to classify the effects of genetic mutations on splicing. Related work has characterized splicing-associated genetic variants (SAVs) based on exome sequencing (Shirashi Genome Res. 2018 August; 28(8): 1111-1125). However, both of these studies merely described the splicing effects of mutations, without recognizing the neoantigenic potential of mis-splicing. Possible neoantigenic effects of mis-splicing mutations have been described in related work by Jayasinghe et al (Cell Rep 2018 Apr. 3; 23(1):270-281 reviewed in Smith et al. (Nature Reviews Cancer 2019 19:465-478)) which made use of a set of 8,656 whole-genome (WGS) and transcriptome sequencing data (short-read RNAseq) to characterize splice-creating mutations. Splice-creating mutations were identified using a novel bioinformatic tool (MiSplice, https://github.com/ding-lab/misplice), which integrates mutation data (from whole genome sequencing) and short-read RNA sequencing data and searches for alternative splice-junctions in the vicinity of genetic mutations. In this work, the prediction of neoantigenic peptides derived from novel splice-junctions is based on the transcript structures available in the RefSeq database. However, the actual expressed transcripts are not taken into account, leading to uncertainties and possible errors in the in silico translation process.

A Novel Approach for Detection of Neoantigens from Mis-Splicing Causing Mutations

Short-read RNA sequencing technology only provides a local view on transcript structure, which is mostly restricted to accurate measurement of the connection between consecutive exons. However, from such individual exon-exon connections, the entire structure of a transcript cannot be reliably determined (Hardwick et al, Front. Genet., 16 Aug. 2019). Given the complex and diverse patterns of transcript isoforms that are expressed in human tissues, an end-to-end sequence of the entire structure of a transcript is required to predict the translated sequences that may emerge from aberrant (mis-spliced) transcripts resulting from genetic mutations in cancer cells. Herein, we propose to use long-read cDNA or mRNA sequencing to detect neoantigens derived from genetic mutations that cause mis-splicing of transcripts in tumor cells. Such mutations also include SNVs resulting in the generation of neoantigens.

In one aspect, the disclosure provides a method for identifying candidate neoantigen sequences (“Frames”). The neoantigen sequences are identified from a tumor sample of an individual afflicted with cancer. As described further herein, such neoantigens may be used to prepare a vaccine or other form of immunotherapy for the treatment of cancer.

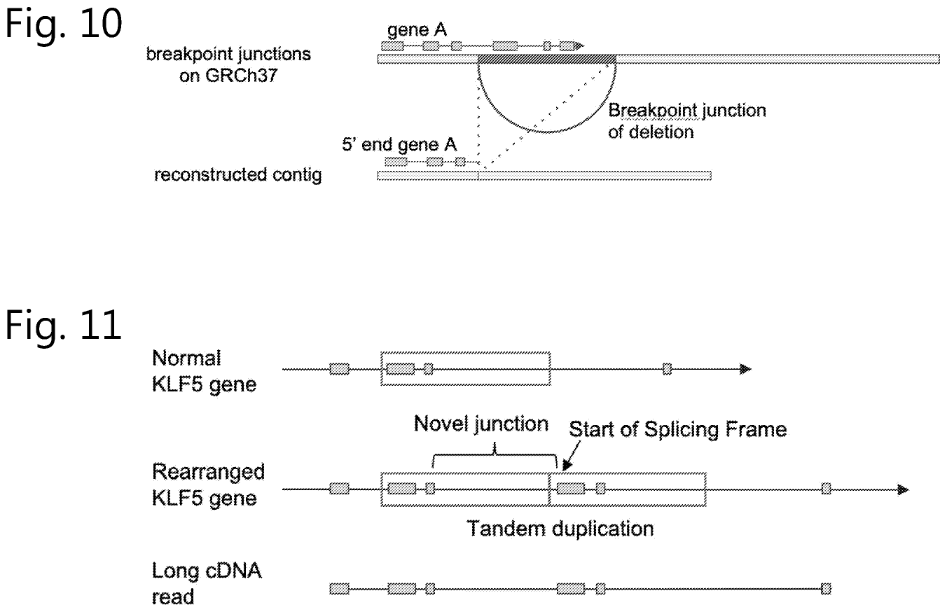

There are two major advantages of using the Framome, i.e. the entire collection of Frames expressed by a tumor, as target of therapeutic anti-cancer vaccines or other forms of immunotherapy. Firstly, Frames are presumed to be the most antigenic neoantigens encoded by tumor genomes as compared to SNV-antigens. As used herein, the term “SNV-antigen” refers to antigens having a single amino acid change. If the potential antigenicity of a tumor were to be expressed as the number of newly encoded amino acids, the Framome covers much, if not the majority of all antigenicity (see FIG. 2, FIG. 9, and FIG. 10 of WO 2021/172990), and thus largely takes the selection process for the best possible neoantigens out of vaccine or immunotherapy development.

Secondly, Frames have an additional advantage over SNV-antigens in regards to HLA-restriction. Small peptides containing a single amino acid change will be presented within the MHC with only few options for a productive presentation, and thus the precise fit of the chosen peptide within the MHC of the specific HLA type of the patient is a point of serious attention. For long viral antigens it has long been concluded that such concern about HLA-matching is of less importance, since the long and entirely foreign (non-self) sequence will be degraded by the proteasome in so many different ways that along the full length of the neoantigen there will always be stretches that match and are thus productive antigens. This also applies to Frames, which are in this respect no different than e.g. the HPV16 and HPV-17 antigens encoded by the Human Papilloma Virus, and which are used successfully for anti-tumor vaccination (Massarelli et al. JAMA Oncol 2019 5:67-73).

While cancer-specific frameshift mutations and SNV-antigens have previously been described, one object of the disclosure is to identify a larger source of potential neoantigens. This includes, e.g., Frames derived from SNVs. Such mutations may, e.g., cause mis-splicing of transcripts in tumor cells or mutate a stop codon, resulting in a tumor specific open reading frame. The present disclosure is not concerned with neoantigens comprising a single amino acid difference resulting from a SNV (i.e., “SNV-antigens”). Rather, only SNVs that result in the expression of novel Frames are encompassed by the present disclosure.

The methods comprise identifying somatic genomic changes in nucleic acid sequences from at least one tumor sample from the individual, wherein the somatic genomic changes result in new open reading frames. In particular, the methods may comprise determining the presence of single nucleotide variants (SNVs), indels, and structural variants that result in tumor specific open reading frames. It is clear to a skilled person that determining the presence of SNVs also includes determining multiple SNVs, or rather multi-nucleotide variants (MNVs). A number of different mechanisms by which genomic mutations can lead to the encoding of novel neoantigen sequences are discussed below.

### Splicing Mutations

Newly synthesized eukaryotic mRNA molecules (i.e., pre-mRNA) are processed by the addition of a 5′ methylated cap and a 3′ poly(A) tail and introns are removed by splicing to form the mature mRNA sequence. Splice junctions are also referred to as splice sites with the 5′ side of the junction often called the “5′ splice site,” or “splice donor site” and the 3′ side the “3′ splice site” or “splice acceptor site.” Donor and acceptor sites are evolutionary conserved and are usually defined by GT and AG nucleotides at the 5′ and 3′ ends of the intron, respectively. After an intron is removed, the exons are contiguous at what is sometimes referred to as the exon/exon junction or boundary in the mature mRNA.

Mutations leading to splice aberrancies of mRNA can be formed by any type of genomic alteration that is found in the genome of cancer cells, e.g., Single nucleotide variants (SNVs), Structural variants (SVs), Short insertions and deletions (indels), and Multi-nucleotide variants (MNVs). Splicing mutations may occur in either introns or exons and may, e.g., disrupt existing splice sites, create new splice sites, or activate cryptic splice sites. In a preferred embodiment, a splice mutation occurs within the coding region (including introns and exons) of a gene. Splice mutations may potentially also occur downstream of the stop codon or at the 3′-end of the gene. Such mutation may induce novel splicing from transcription that continues past the gene 3′ end (read through transcription). Mutations at donor and acceptor sites as well as within 20 nucleotides of said sites are a large source of splicing-mutations. Mutations occurring more than 20 bp away from the nearest intron/exon junction are referred to herein as “deep intronic mutations”. While most deep intronic mutations are silent, some affect canonical and auxiliary splicing cis-elements or generate cryptic GT-AG dinucleotides.

Whether a mutation is directly causal to a splice aberrancy (i.e. a cis-effect), is primarily determined by the genomic proximity of the mutation to the mis-spliced RNA junction. Hence, a combination of whole genome sequencing and RNA (or cDNA) sequencing is required to effectively identify mutations causing mis-splicing, as well as the exact effects of the mis-splicing on mRNA structure. Herein, we characterize three types of exemplary mutations causing mis-splicing:

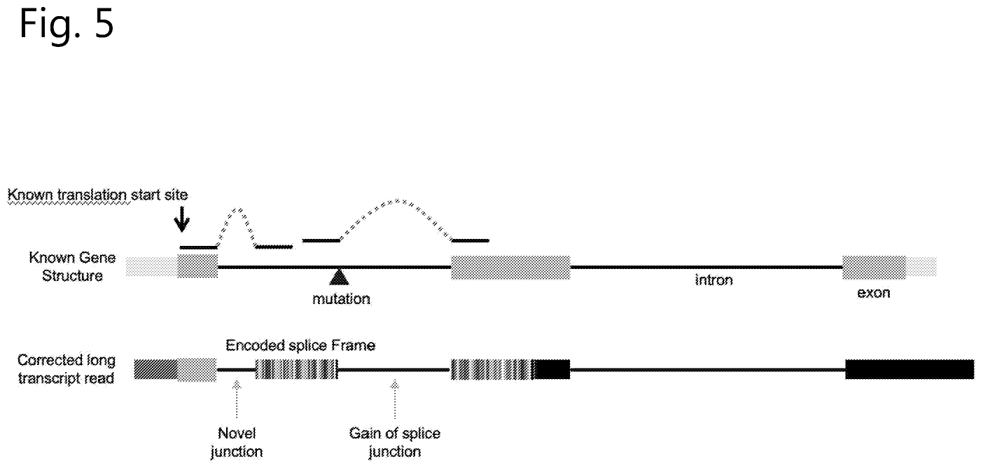

Firstly, small mutations (e.g., indels, SNVs, MNVs) that result in gain of a splice site (gain-of-splice [GOS] mutations, FIG. 5). Such mutations create a splice-donor or splice-acceptor site, thereby creating a novel splice-junction in the RNA. In preferred embodiments, such mutations are within 50 bp of a novel splice-junction, more preferably within 20 bp.

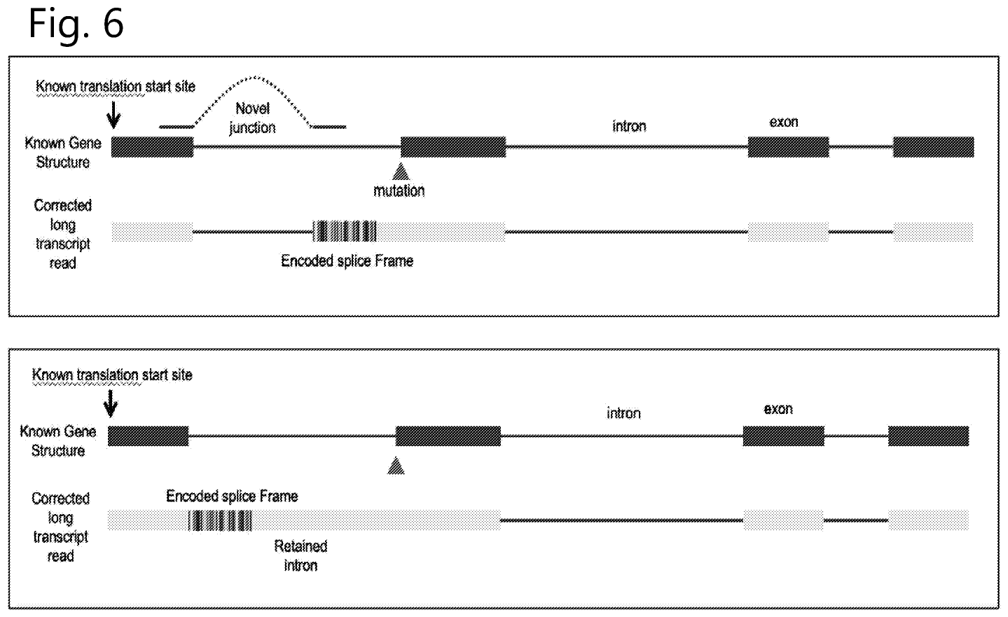

Secondly, small mutations (indels, SNVs, MNVs) that result in loss of a splice site (loss-of-splice [LOS] mutations, FIG. 6). Such mutations disrupt a known splice-donor or splice-acceptor site, thereby leading to the use of alternative splice sites by the splicing machinery. This may cause, amongst others, exon skipping, exon extension, or intron retention. In preferred embodiments, such mutations disrupt GT or AG consensus splice sequences or are within 50 bp, preferably 20 bp of said sequences.

Both GOS and LOS mutations have been described in prior work (Jung et al, Oncogene volume 40, pages 1347-1361 (2021); Jayasinghe et al, Cell Reports VOLUME 23, ISSUE 1, P270-281.E3, Apr. 3, 2018; Shiraishi et al, Genome Research 2018 August, 28(8): 1111-1125).

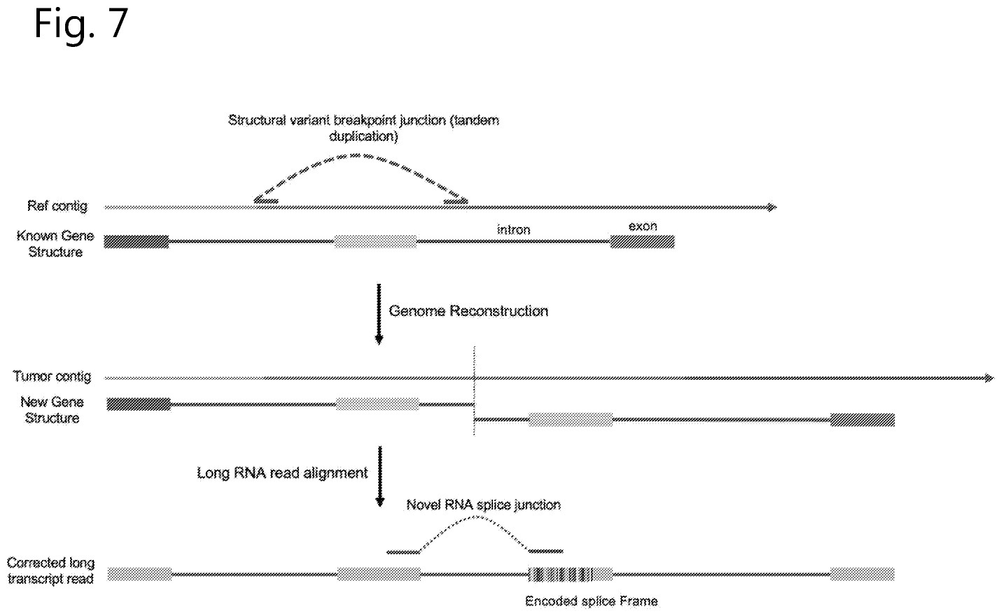

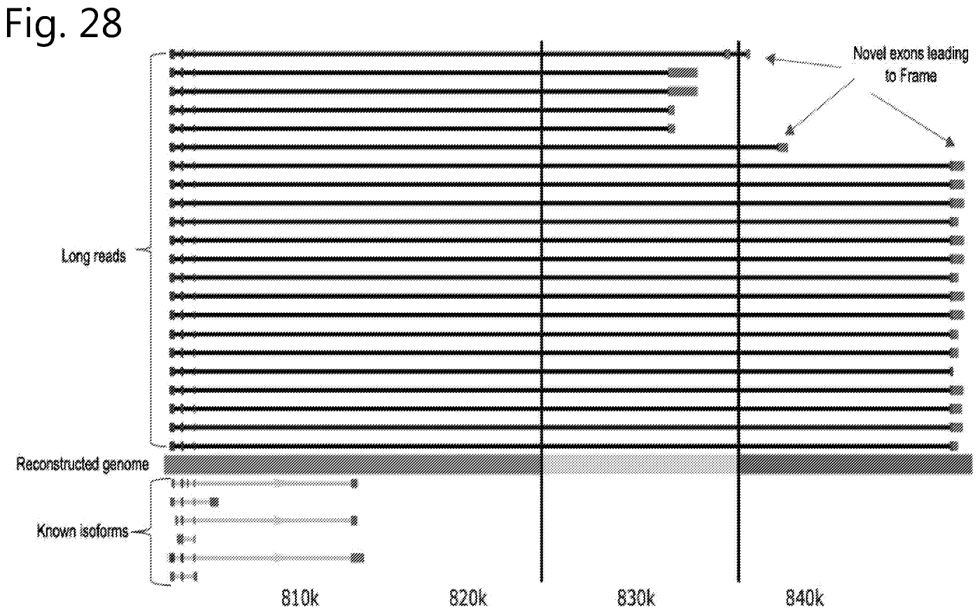

Thirdly, structural variants (SVs) that result in the rearrangement of splice sites and/or the exon-intron structure of a mRNA (FIG. 7 and FIG. 28). Structural variations are DNA rearrangements, which encompass at least 50 bp although such variations are normally around 1 kb or larger in size. SVs include, e.g., deletions, duplications, insertions, inversions, and translocations. See for a review Mahmoud et al. Genome Biology 2019 20:246. While neoantigens caused by SVs are relevant in the majority of tumors, this source of antigenicity is especially relevant in cancers having complex chromosome rearrangements such as chromothripsis, chromoplexy and chromoanasynthesis. SVs causing DNA rearrangements leading to novel Frames and subsequent formation of neoantigens are discussed in more detail further herein.

Exemplary algorithms for identification of mutations causing mis-splicing of mRNA and the neoantigenic sequences caused by such mutations are described below. A skilled person recognizes that other methods or variations of the method may also be used. As will be appreciated by a skilled person, such algorithms involve the use of computers and computer programs.

For gain-of-splice-site (GOS) mutations the preferred steps in the algorithm (herein referred to as A1) can be described as follows.
a) Mapping the sequences obtained from whole genome sequencing of a tumor sample and a corresponding healthy control sample to a human reference sequence to identify somatic genomic variations in the tumor sample as described further herein. In a preferred embodiment the genomic sequences are mapped to a reference human genome sequence (GRCh37, GRCh38, or the like). This step also distinguishes germline genetic variations (identified from the healthy tissues) from tumor-specific genetic variations (identified from the tumor tissue) as discussed herein. Mapping can be accomplished using tools known to the skilled person, such as Burrows Wheeler Alignment or the like (Li & Durbin, Bioinformatics. 2009 Jul. 15; 25(14): 1754-1760).
b) Identification of somatic mutations in the aligned genome sequencing data using existing approaches that use the sequences of multiple reads at each genomic locus to infer the presence of a mutations (Koboldt et al, Genome Research 2012 March; 22(3):568-76; Alioto et al, Nature Communications volume 6, Article number: 10001 (2015)). Comparison of the aligned sequences for tumor and normal tissue samples determines whether a mutation is somatic (i.e. tumor-specific). Potential GOS mutations are selected from the entire collection of somatic mutations identified in a tumor genome, based on their presence within a gene and distance from known splice sites (e.g., as based on annotations in databases).
c) A subsequent step involves aligning the long-read cDNA (or RNA) sequences, and optionally short-read sequences or long-read consensus sequences, to the human reference genome sequence. The mapping can be done using existing software known in the art, including but not limited to STAR (Dobin et al, Bioinformatics, Volume 29, Issue 1, January 2013, Pages 15-21) and minimap2 (Li, Bioinformatics, Volume 34, Issue 18, 15 Sep. 2018, Pages 3094-3100). The mapping places subsequences of the cDNA sequences on to the reference genome in a process known as split-alignment. The splits between aligned subsequences, typically represent splice junctions.
d) Determining the presence of aberrant splice-junctions in aligned RNA sequencing data for genes containing small somatic mutations of any type as described herein (e.g., SNVs, MNVs, and indels). Identification of aberrant splice-junctions preferably involves comparison of the measured splice-junctions in a tumor sample to sets of known splice-junctions identified from unrelated samples, such as healthy tissue samples or unrelated tumor samples. Preferably, the splice-junctions described by the GTEX consortium (available at https://gtexportal.org/home/publicationsPage) are used to remove known splice-junctions measured in a tumor sample. Preferably, splice-junctions described in a human genome database, such as Ensembl (available on the world wide web at ensembl.org) may be used to remove known junctions and identify tumor-specific junctions near genetic mutations in genes. Preferably, splice-junctions unique for a tumor sample should be observed in both long-read and short-read RNA sequencing data. RNA splice-junctions are preferably in the vicinity of a GOS mutation, e.g., within 50 bp of a GOS mutation, more preferably within 20 bp of a GOS mutation.
e) Determining sequences of the full-length RNA transcripts resulting from the GOS mutations. The present disclosure provides that when the transcription/splicing machinery encounters a GOS mutation, it may seek a new splice site in the vicinity of the mutation, resulting in an RNA transcript with a novel open reading frame. Long RNA sequencing reads described herein can be used to determine the sequence of the new RNA transcripts that are a result of a GOS mutation. The long RNA sequence reads may be generated using one or more methods for obtaining high-accuracy long transcript sequences, including but not limited to consensus sequencing, as described herein.
f) Determining the predicted amino acid sequences encoded by the full-length transcripts of d) as further described herein. The long transcript sequences are compared to existing transcript structures from known gene annotation databases (Ensembl or the like), and the annotated translation start site is used as a starting point for the in silico translation process. This method provides an improved pipeline for determining tumor neoantigens, in particular for neoantigens resulting from mutations causing mis-splicing. This method can also be used to select for such tumor neoantigens (referred to herein as Splice Frames) by:
g) Selecting, as candidate neoantigen sequences, sequences comprising at least 9 contiguous amino acids of the predicted amino acid sequence of e), wherein at least four of the contiguous amino acids are not encoded in the germline genome of the individual, as further described herein.

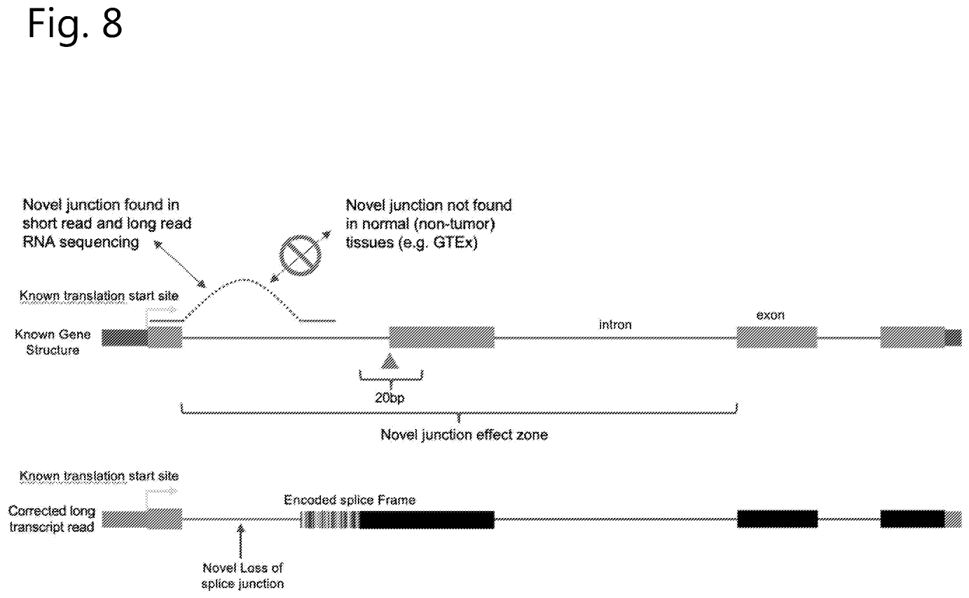

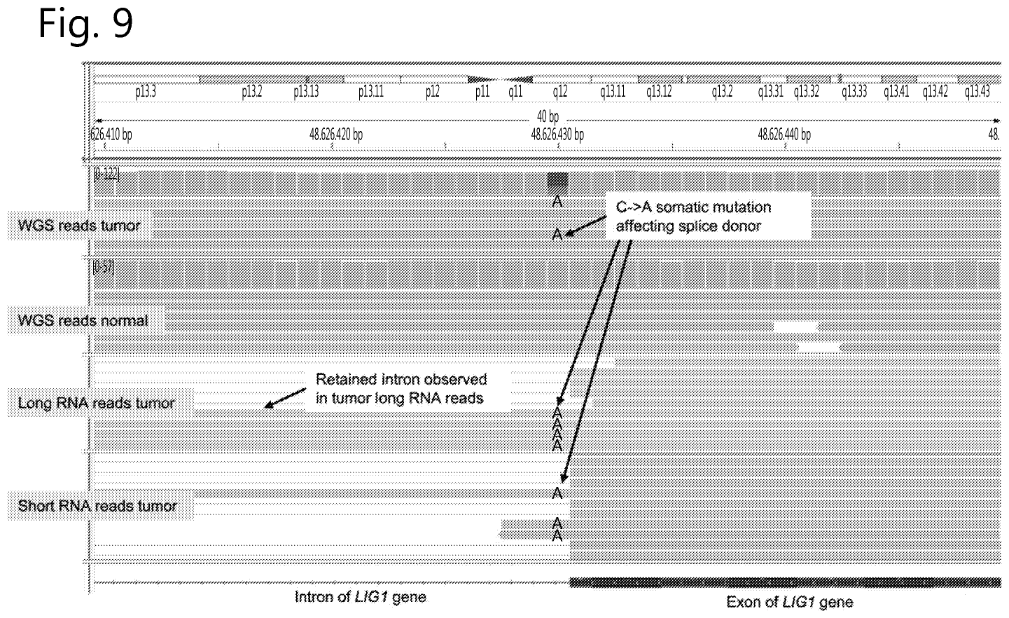

For loss-of-splice-site (LOS) mutations the preferred steps in the algorithm (herein referred to as A2) can be described as follows.
a) Mapping the sequences obtained from whole genome sequencing of a tumor sample and a corresponding healthy control sample to a human reference sequence to identify somatic genomic variations in the tumor sample as described further herein, as described in A1 step a) above.
b) Identification of somatic mutations in the aligned genome sequencing data, as described in A1 step b) above. LOS mutations are a subset from somatic mutations identified in the tumor sample and are typically confined to mutations in the vicinity of known splice sites. Preferably mutations within 50 bp of a known splice site, more preferably mutations within 20 bp of a known splice site.
c) A subsequent step involves aligning the long-read cDNA (or RNA) sequences to the human reference genome sequence, as described in A1 step c) above.
d) Determining the presence of aberrant splice-junctions in aligned RNA sequencing data, as described in A1 step d) above. RNA splice-junctions are preferably in the vicinity of a LOS mutation, i.e. between the exon preceding the LOS mutation and the exons after the LOS mutation (referred to as the effect zone) (FIG. 8). In some embodiments, the LOS mutation may lead to a retained intron (FIG. 6). A retained intron does not introduce a new splice-junction, because it results from a lack of splicing between two neighboring exons. Retained introns have previously been described and occur occasionally in normal tissues even without the presence of splice mutations (e.g. Li et al, BMC Genomics volume 21, Article number: 128 (2020)). Hence, the causal relationship between a retained intron and a splice donor or acceptor can be assessed by means of the presence of a LOS mutation in the RNA reads containing the retained intron (FIG. 9).
e) Determining sequences of the full-length RNA transcripts resulting from the LOS mutations, as described in A1 step e) above.
f) Determining the predicted amino acid sequences encoded by the full-length transcripts of d), as described in A1 step f) above. This method can also be used to select for such tumor neoantigens (referred to herein as Splice Frames) by:
g) Selecting, as candidate neoantigen sequences, sequences comprising at least 9 contiguous amino acids of the predicted amino acid sequence of e), wherein at least four of the contiguous amino acids are not encoded in the germline genome of the individual, as further described herein.

For structural variants (SVs) in the tumor genome, the effects on mRNA splicing can be diverse and not readily predicted based on the mutation in the DNA. An exemplary algorithm (herein referred to as A3) to detect splicing aberrancies caused by SVs can be defined as follows:
a) Mapping the sequences obtained from whole genome sequencing of a tumor sample and a corresponding healthy control sample to a human reference sequence to identify somatic genomic variations in the tumor sample, as described in A1 step a) above.
b) Identification of somatic SVs in the aligned genome sequencing data. Specific software is available for using read alignments for identification of large structural genomic rearrangements, including but not limited to deletions, duplications, inversions, insertions and translocations. An example of such software is GRIDSS, which uses split-read and read-pair mappings and retrieves the sequences of genomic rearrangement breakpoint-junctions through assembly of discordantly mapping sequence reads (Cameron et al. Genome Res 2017 27:2050-2060). Other existing software tools are Delly (Rausch et al. Bioinformatics 2012 28:i333-i339), or Manta (Chen et al. Bioinformatics 2016 32:1220-2), which are based on similar principles. An overview of the methods to identify genomic rearrangements in cancer genomes can be found in the paper by Kosugi et al (Kosugi et al. Genome Biol 2019 20:117). SVs having a breakpoint within the coding region of a gene or within 100 kb downstream of the coding region are selected from the entire set of somatic SVs identified specifically in a tumor sample (as compared to a corresponding normal tissue specimen).
c) Generating in silico a reconstructed tumor-specific reference genome comprising the identified somatic structural genomic variations. As will be understood by the skilled person, it is not necessary to generate a complete tumor-specific reference genome. Rather, contigs which span the structural genomic variations can be generated (see, e.g., FIG. 10). Such contigs are generally around 100 kb but can be longer, e.g., 300-400 kb. Longer contigs may be useful in genomic regions which comprise a large number of re-arrangements. The reconstructed tumor-specific reference genome contigs can be generated by any method known to a skilled person. For example, the genomic DNA segments from the reference human genome sequence can be joined based on the information on breakpoint junctions derived from the WGS (e.g., using SV variant calling). Alternatively, the WGS data comprising the SVs may be directly used in an assembly algorithm to generate assembled contigs covering the rearranged segments.
d) Aligning the RNA sequences to the reconstructed tumor-specific reference genome. This step is useful when mapping RNA sequencing data to the genome. The cancer tumor often comprises complex rearrangements which complicate the mapping of RNA sequences, in particular as the order and orientation of exonic sequences in the tumor genome may be different than in the human reference genome. As shown in FIG. 11, mapping short-read RNA sequencing data to the human GRCh37 reference failed to identify transcript reads derived from an intragenic tandem duplication in the KLF5 gene. However, the novel RNA junctions and transcript structure is found when mapping long-read RNA sequencing reads to a reconstructed tumor-specific contig. The mapping can be done using existing software known in the art, including but not limited to STAR (Dobin et al, Bioinformatics, Volume 29, Issue 1, January 2013, Pages 15-21) and minimap2 (Li, Bioinformatics, Volume 34, Issue 18, 15 Sep. 2018, Pages 3094-3100). The mapping places subsequences of the cDNA sequences on to the reference genome in a process known as split-alignment. The splits between aligned subsequences, typically represent splice junctions.
   In some embodiments, this step is an iterative process comprising mapping of long-read sequencing data (and optionally short-read sequencing data) to the reconstructed contigs. The short-read data can be used to polish (i.e., correct) the long-read data. The long-read data is particularly useful to determine the correct splicing pattern of the transcripts. In turn, the short-read data precisely determine each separate splice-junction, enabling polishing of the long RNA sequencing reads and the splice-junction patterns identified therein. Long-read data also allows the identification of multiple, alternative transcripts (e.g. Hu et al, Genome Biology volume 22, Article number: 182 (2021)).
e) Determining the presence of aberrant splice-junctions in aligned RNA sequencing data for genes containing SVs as described herein. Identification of aberrant splice-junctions preferably involves comparison of the measured splice-junctions in a tumor sample to sets of known splice-junctions identified from unrelated samples, such as healthy tissue samples or unrelated tumor samples. Preferably, the splice-junctions described by the GTEX consortium (https://gtexportal.org/home/publicationsPage) are used to remove known splice-junctions measured in a tumor sample. Preferably, splice-junctions described in a human genome database, such as Ensembl (www.ensembl.org) may be used to remove known junctions and identify tumor-specific junctions near genetic mutations in genes. Preferably, splice-junctions unique for a tumor sample should be observed in both long-read and short-read RNA sequencing data. RNA splice-junctions are preferably in the vicinity of a SV breakpoint, i.e. between the exon preceding the SV breakpoint and the exons after the SV breakpoint (referred to as the effect zone) (FIG. 8). In some embodiments, the SV may lead to a retained intron (FIG. 6, FIG. 9). A retained intron does not introduce a new splice-junction, because it results from a lack of splicing between two neighboring exons. Retained introns are described in prior art and occur occasionally in normal tissues even without the presence of splice mutations (e.g. Li et al, BMC Genomics volume 21, Article number: 128 (2020)). Hence, the causal relationship between a retained intron and a splice donor must be assessed by means of the presence of an SV in the RNA reads containing the retained intron (FIG. 9).
f) Determining sequences of the full-length RNA transcripts resulting from the SV. The present disclosure provides that when the transcription/splicing machinery encounters a SV that disrupt a known splice site, it may seek a new splice site in the vicinity of the mutation, resulting in an RNA transcript with a novel open reading frame. Alternatively, an SV may introduce a new splice site without disruption of a known splice site. Long RNA sequencing reads described herein, can be used to determine the sequence of the new RNA transcripts that are a result of an SV. The long RNA sequence reads may be generated using one or more methods for obtaining high-accuracy long transcript sequences, including but not limited to consensus sequencing, as described herein.
g) Determining the predicted amino acid sequences encoded by the full-length transcripts of d), as described in A1 step f) above. This method can also be used to select for such tumor neoantigens (referred to herein as Splice Frames) by:
h) Selecting, as candidate neoantigen sequences, sequences comprising at least 9 contiguous amino acids of the predicted amino acid sequence of e), wherein at least four of the contiguous amino acids are not encoded in the germline genome of the individual, as further described herein.

### Frameshift Mutations

The methods described herein may also be used to identify intragenic frameshift mutations in polypeptide encoding sequences, wherein the mutation results in a change of the reading frame of said polypeptide encoding sequence. Such neoantigens (i.e., Frames) result from insertions and deletions within coding exons of a single gene. As is well-known to a skilled person, a “frame shift mutation” is a mutation causing a change in the frame of the protein, for example as the consequence of an insertion or deletion mutation (other than insertion or deletion of 3 nucleotides, or multitudes thereof). Such frameshift mutations result in new amino acid sequences in the C-terminal part of the protein. These new amino acid sequences (encoded by the new open reading frame) generally do not exist in the absence of the frameshift mutation and thus only exist in cells having the mutation (e.g., in tumor cells and pre-malignant progenitor cells).

Frameshift mutations can be identified based on the exome from the tumor, although whole genome sequencing may be preferred. Expression of relevant Frames resulting from frameshift mutations can be determined by RNA sequencing. Exemplary methods for identifying frameshift mutations and identifying neoantigens resulting from said mutations are also described in WO2021/172990.

### Stop Codon Mutations

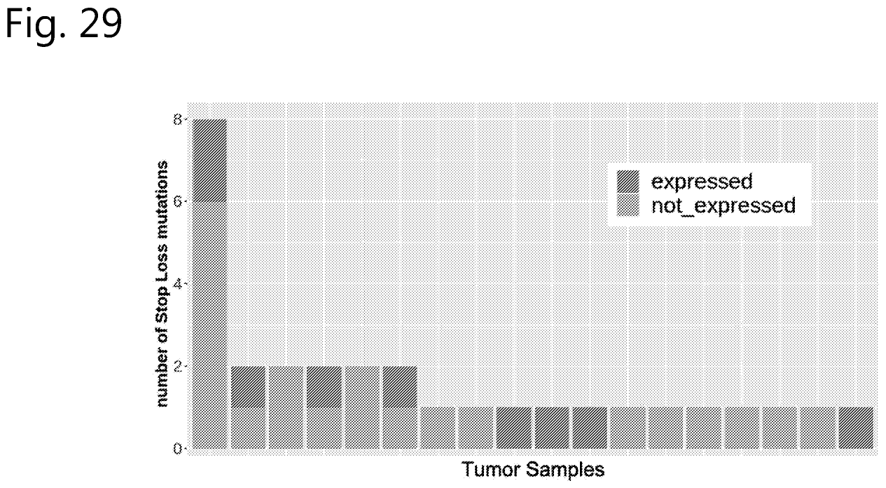

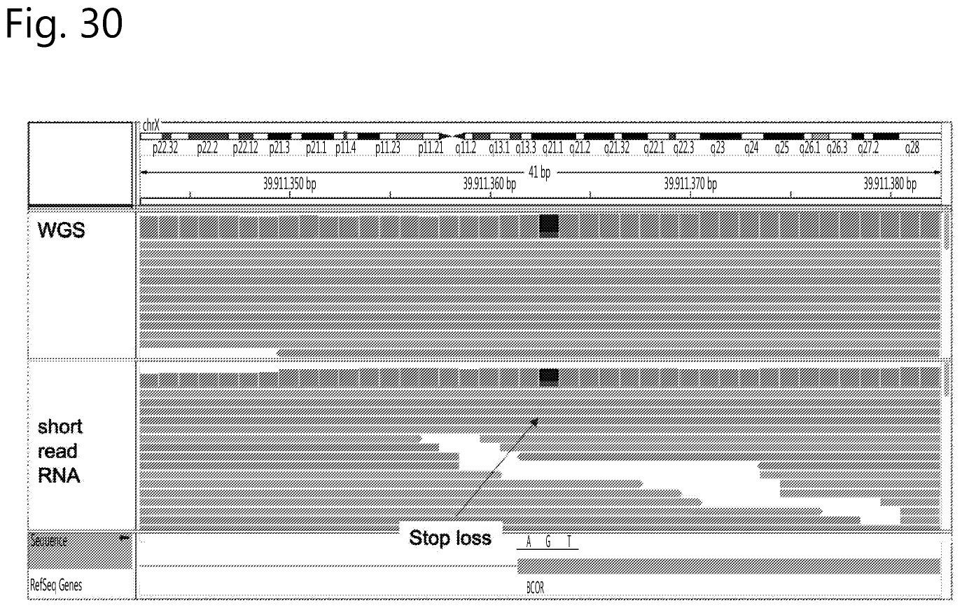

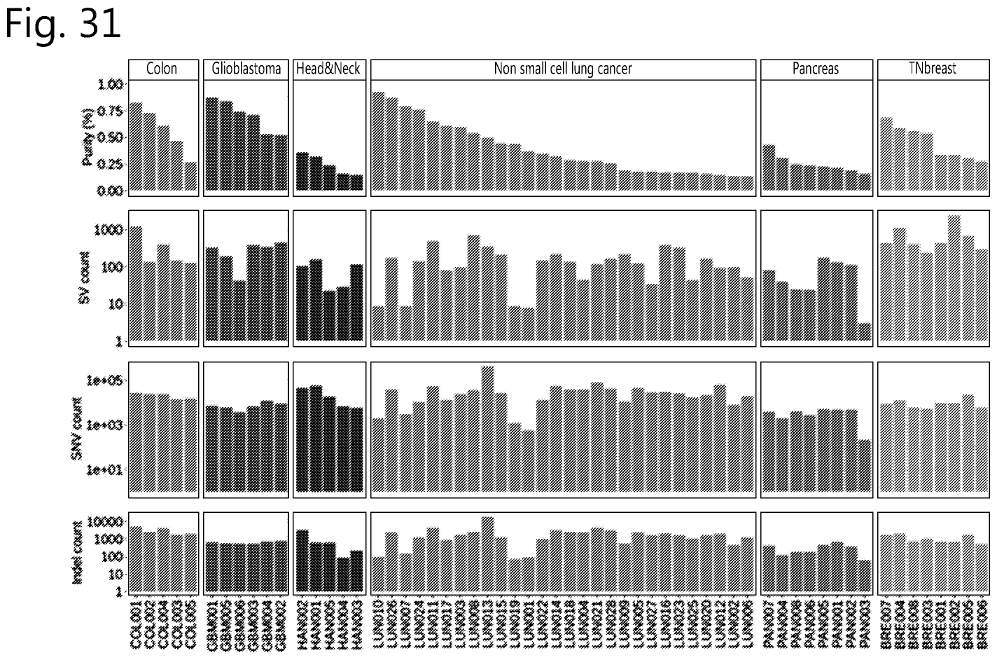

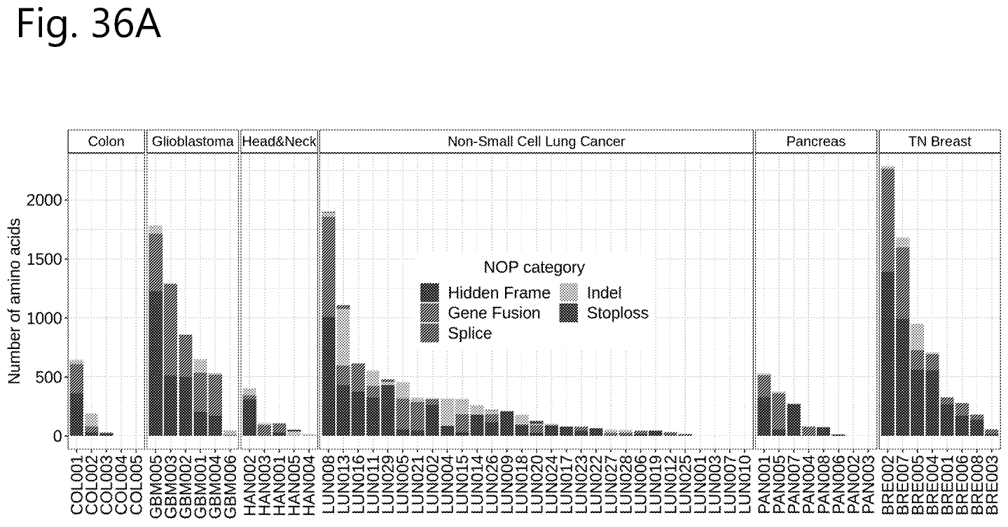

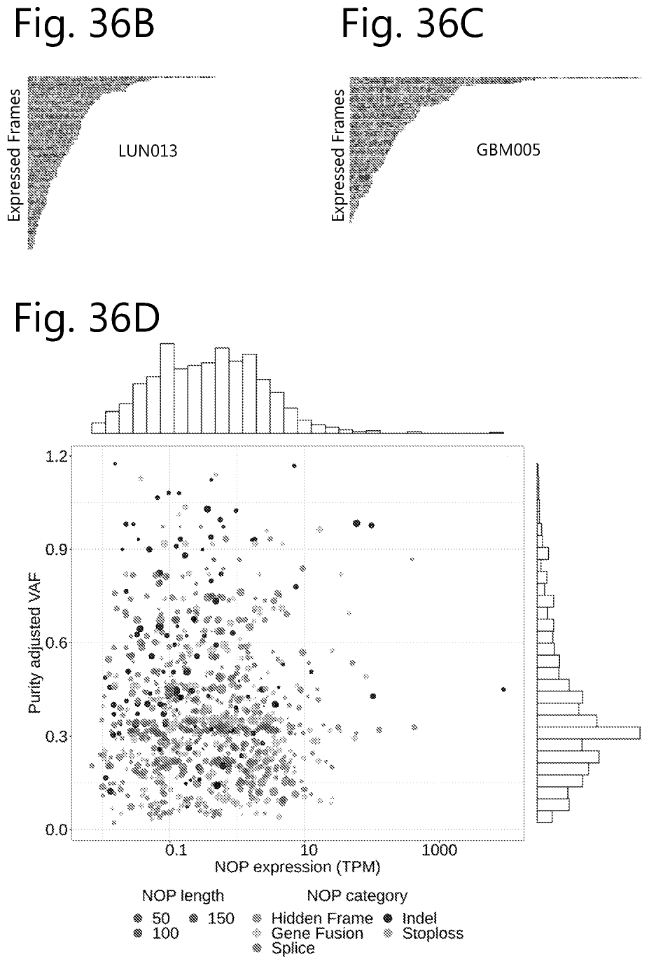

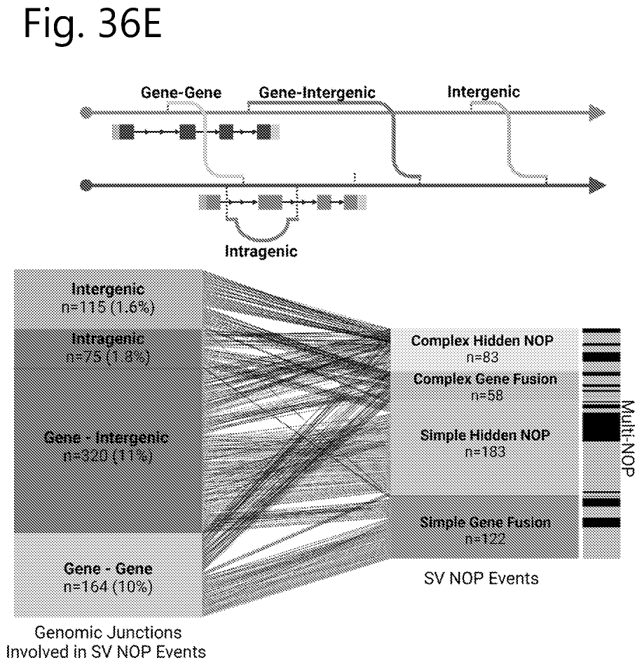

Another type of mutation that leads to novel Frames are mutations in stop codons. For example, a SNV can result in the mutation of a stop codon to a codon encoding an amino acid (FIG. 29, FIG. 30, FIG. 36). A novel Frame is generated comprising the new codon as well as downstream sequences until the occurrence of the next stop codon.

Such mutations can be identified based on the exome from the tumor, although whole genome sequencing may be preferred. Expression of relevant Frames resulting from such mutations can be determined by RNA sequencing. Expression analysis involves the identification of the stop codon mutation in individual (long) poly-adenylated and 5′-capped transcript reads. Those transcript reads containing the stop codon mutation are then subjected to in silico translation as outlined herein.

### Structural Variations (SV)

Another type of mutation that leads to novel Frames are DNA rearrangements, in particular structural variations. SVs may result in DNA gain (e.g., copy number variations, such as tandem duplications), DNA loss (e.g., deletions which may disrupt gene function), as well as balanced rearrangements that do not involve loss or gain of chromosomal sequence (e.g. inversions, reciprocal translocations). Each of the possible SV types may possibly lead to new open reading frames.

We have found that in many cases during transcription of a ‘proper’ gene that spans a genomic breakpoint-junction which connects the gene to another piece of the genome, the transcription machinery will seek and find a preferred place for transcription termination and polyadenylation of the RNA and the splicing machinery will seek and find splice sites. The result is a fully processed and translatable mRNA, complete with 5′-CAP and poly-(A)-tail. In our results, we observe that there is often either one or only a few dominant mRNA variants that emerge from the process of transcription across somatic genomic breakpoint-junctions and RNA-processing. These variants result in new open frames and are a large source of tumor antigenicity.

### Out of Frame Fusions

One type of structural variant refers to DNA rearrangements resulting in new junctions of DNA sequences, wherein the rearrangement results in the fusion of at least part of the coding strand of a first gene to at least part of the coding strand of a second gene. Alternatively, the rearrangement results in an intragenic rearrangement, such as an intragenic deletion or (tandem) duplication, thereby creating an intra-genic fusion, between the upstream (5′) part of a gene and the downstream (3′) part (including the poly-(A) signal). In particular, the DNA rearrangement results in a change of the reading frame of a polypeptide encoding sequence, herein referred to as ‘out of frame gene fusions’.

In some embodiments, such mutations result in the fusion of at least part of the coding strand of a first gene to at least part of the coding strand of a second gene (i.e., intergenic genomic rearrangement). The reading frames of the first and second gene are different at the position of the junction in the mRNA, resulting in a novel open reading frame. Such mutations may result from various DNA rearrangements including but not limited to inversions, deletions, or translocations. As is understood by a skilled person, the coding strand (i.e., sense strand) of a gene is the strand comprising the sequence corresponding to the mRNA sequence. Out of frame gene fusions may encode the entire protein corresponding to the first gene or only a part thereof. The out of frame fusion with the coding strand of the second gene results in a Frame (i.e., neoORF). Given that for most genes the introns are much larger than the exons, in some embodiments the mutation results from the fusion of two genes with a genomic junction that maps for each gene within an intron. If splicing were to proceed using the splice sites of the parental genes, the splice product may fuse the downstream partner within the frame of the upstream partner, which can lead to a neoORF. In preferred embodiments, the mutations result in a nucleic acid sequence encoding an mRNA comprising a start codon encoded by the first gene and a poly-(A) signal encoded by the second gene.

In some embodiments, the mutations are intragenic genomic rearrangements which result in a neoORF. For example, such mutations may lead to the fusion of exons of the same gene having different reading frames. Intragenic genomic rearrangements are known to a skilled person and include, but are not limited to, intragenic deletions, intragenic tandem duplications, intragenic dispersed duplications, intragenic inverted duplications, intragenic insertions, and intragenic inversions.

In some embodiments, the said intragenic genomic rearrangements lead to a rearrangement of the natural exon-intron structure of a known gene in the human genome. There are multiple types of rearrangements that can affect the proper splicing of a gene, including, for example, deletions, duplications, inversions, and insertions.

### Hidden Frames

Another type of structural variant refers to DNA rearrangements resulting in new junctions of DNA sequences, wherein the rearrangement results in the fusion at least part of the coding strand (most often an intronic sequence, but exonic or other sequence is also possible) of a first gene to a second sequence selected from intergenic non-coding DNA or to the noncoding strand of a second gene. The fusion results in the coding strand of the first gene being 5′ of the second sequence. Unlike ‘out of frame fusions’ mutations discussed above which fuse two genetic sequences having the same orientation (i.e., the coding strands from two genes are fused), ‘Hidden Frame’ mutations refer to the fusion of a first gene with a second sequence that does not encode for a gene or does not encode for a gene in the same orientation as the first gene. We refer to these neoantigens as “Hidden Frame Neoantigens” since they cannot be accurately predicted based solely on the genomic DNA sequence because the transcription termination and splicing after fusion of two DNA segments is inherently unpredictable.

This second sequence may be (intergenic) non-coding DNA. (Intergenic) non-coding DNA includes DNA which is not predicted to encode a protein. Such non-coding DNA includes repetitive DNA, as well as DNA that regulates expression (e.g., promoters, enhancer elements, etc) and DNA that encodes non-coding RNA (ncRNA). ncRNA refers to RNA that is not translated into protein and includes tRNA, rRNA, microRNAs, etc. See, e.g., FIG. 8 of WO2021/172990 as an exemplary embodiment. The second sequence may be the noncoding strand of a second gene.

See Example 7 of WO2021/172990, which is incorporated by reference herein, for an exemplary embodiment of for carrying out the FramePro method for identifying tumor neoantigens.

In preferred embodiments, the Hidden Frame mutations result in a nucleic acid sequence encoding an mRNA comprising a start codon encoded by the first gene and a poly-(A) signal encoded by the second sequence. The poly-(A) signal encoded by the second sequence may also be referred to as a ‘cryptic’ polyadenylation signal since the poly-(A) signal (without the mutation) is not normally associated with mRNA or a protein encoding sequence.

Another example of a Hidden Frame is the result of a genomic rearrangement outside of a gene resulting in the change of the genomic sequences flanking the 3′ end of a gene. The altered genomic sequences flanking the 3′ end of a gene may contain cryptic splicing signals, which lead to new mRNA structures. In such an embodiment, the SV breakpoint resides downstream of the stop codon, e.g. within 100 kb downstream of the stop codon. Such rearrangement fuses the coding strand of a first gene to a second sequence. The second sequence may be any sequence, e.g., intergenic non-coding DNA or the coding or noncoding strand of a second gene. The mutation results in novel splicing and the expression of a tumor specific open reading frame. An example of such Hidden Frames is depicted in FIG. 28.

As is known to a skilled person, messenger RNA is polyadenylated with the addition of a 3′ poly-(A) tail. The poly-(A) tail is involved in a number of processes including nuclear export and protein translation. Polyadenylation signals near the 3′ end of mRNA direct the cell machinery to add a poly-(A) tail. The most common polyadenylation signal on the RNA is AAUAAA. However other variants also exist.

The sequences of such signals and methods for identifying such signals in nucleic acid sequences are well-known in the art and can be predicted by a number of different in silico methods. For example, the genomic sequence of the non-coding second sequence may be analyzed by a sequencing method, such as Illumina sequencing, or the like. In a second step the entire sequence assembled from individual sequencing reads may be screened in silico for the presence of known polyadenylation motifs/signal, e.g. using pattern matching, such as regular expressions, known by persons skilled in the art. Alternatively, one can experimentally test the presence of a poly-(A) tail at the 3′ end of an mRNA, by selecting the mRNAs by binding them to polyT oligonucleotides and removing all non-bound RNA. Using such selected mRNAs for high-throughput sequencing, preferably long-read sequencing, for example Nanopore sequencing, one can determine the sequences of all polyadenylated mRNAs in a tumor specimen or tumor cell. In preferred embodiments, the methods comprise selecting poly(A)-RNA. Such methods do not require a priori any knowledge of whether the corresponding encoding nucleic acid sequence comprises a poly(A) signal.

As is known to a skilled person, messenger RNA normally comprises a five-prime cap (5′ cap). In eukaryotes, mRNA is “capped” at the 5′ end with 7-methylguanylate during transcription. Methods for selecting and enriching for 5′ capped RNA are known in the art. For example, the TeloPrime Full-Length cDNA Amplification Kit V2 from Lexogen uses Cap-Dependent Linker Ligation (CDLL) and long reverse transcription (long RT) technology to select full-length RNA molecules that are both capped and polyadenylated. Other methods include the use of a mRNA 5′ Cap Structure Affinity Column Preparation as described in U.S. Pat. No. 6,187,544B1.

A skilled person will recognize that all classes of mutations discussed above may not be present in a particular tumor or that not all classes of mutations will be represented in the RNA of a tumor sample. However, the methods are suitable for identifying the presence or absence of such mutations.

Neoantigens resulting from many of the classes of mutations described above cannot be predicted based solely on the DNA sequence. This is particularly relevant for neoantigens resulting from structural rearrangement. In a preferred embodiment, the method of the disclosure combines whole genome sequences with whole full-length transcriptome sequencing (in order to obtain the full-length sequence of intact mRNA). Preferably, the method uses three datasets:
1) whole genome sequencing to identify somatic structural variants from a tumor
2) full-length mRNA sequencing (usually between 20-100 million reads) from the tumor, preferably mRNAs having a 5′ cap and poly-A tail and
3) (short) cDNA sequencing reads from the tumor.

In some embodiments, the candidate neoantigen sequences described herein may be identified by a method, comprising
a) performing whole genome sequencing of a tumor sample and a healthy sample from the individual,
   optionally performing long-read whole genome sequencing of a tumor sample and a healthy sample from the individual,
b) performing long-read RNA sequencing on RNA or long-read sequencing on the corresponding cDNA from at least one tumor sample, preferably wherein RNA is poly-(A) selected mRNA and/or 5′ cap containing mRNA;
c) identifying structural genomic variations in the tumor sample, using the whole genome sequencing data from (a);
d) determining the sequences of full-length RNA transcripts encoded by nucleic acid sequences comprising (or overlapping with) the somatic structural genomic variations;
e) determining the (predicted) amino acid sequences encoded by the full-length transcripts. Neoantigens useful for treatment comprise at least 8, preferably at least 9 contiguous amino acids of the (predicted) amino acid sequences, wherein at least one, two, three, or preferably at least four of the contiguous amino acids are not encoded in the germline genome of the individual.

A skilled person can readily identify genomic changes in a sequence. While partial sequencing/targeted/exome sequencing is often used on tumor tissue, such methods primarily identify single nucleotide variants (SNVs), or other small genetic variations present in (protein) coding sequences of the genome. In contrast, the present methods rely on whole genome sequencing.

In order to determine whether such genomic changes are somatic, the sequences obtained from the tumor sample can be compared to sequences from non-tumor tissue (also referred to herein as a “healthy sample”) of the patient, e.g., blood. The comparison of tumor sequences and sequences from non-tumor tissue are often compared via mapping of the sequences to a human reference genome, as is known by a person skilled in the art.

In some embodiments, the method further comprises performing whole genome sequencing of a healthy sample (i.e., a non-tumorous sample) from the individual. Whole genome sequencing is generally performed using a short-read sequencing library (e.g., shotgun sequencing with paired-end sequencing reads of 2×150 bp). In preferred embodiments, the method comprises performing long-read whole genome sequencing on the tumor sample, either alone or preferably in combination with short-read whole genome sequencing. Long-read sequencing is especially useful for tumors having complex genomic rearrangements. Long-read sequencing may also be used to sequence a healthy sample. As described further herein, long-read sequencing methods are often referred to as “third generation sequencing” and include systems from Pacific Biosciences and Oxford Nanopore technologies. As a skilled person will recognize, when using highly accurate long-read sequencing techniques, short-read sequencing is redundant.

The methods identify somatic genomic changes that result in new open reading frames. The new open reading frames are not present in the germline genome of the individual. In some embodiments, the methods comprise comparing the nucleic acid sequences from at least one tumor sample with reference sequences. Sequence comparison can be performed by any suitable means available to the skilled person. Indeed, the skilled person is well equipped with methods to perform such comparison, for example using software tools like BLAST and the like, or specific software to align short or long sequence reads.

In some embodiments, the reference sequences are obtained from sequencing healthy tissue from said individual. A comparison of the sequences between a tumor sample and healthy tissue will identify somatic genomic mutations present in the tumor sample. This comparison often makes use of a comparison of the tumor and the healthy tissue sample to a reference human genome sequence (GRCh37, GRCh38, or the like). The differences with respect to the reference human genome sequence are subsequently compared between tumor and healthy tissue. This provides a list of genetic changes that solely occur in the tumor genome, often referred to as somatic genetic changes. In some embodiments, the reference sequence is a human reference genome such as GRCh37 (the Genome Reference Consortium human genome (build 37) date of release February 2009) or GRCh38 the Genome Reference Consortium human genome (build 38) date of release December 2013.

Analysis of sequence reads and identification of mutations will occur through standard methods in the field. For sequence alignment, aligners specific for short or long reads can be used, e.g. BWA (Li and Durbin, Bioinformatics. 2009 Jul. 15; 25(14):1754-60) or Minimap2 (Li, Bioinformatics. 2018 Sep. 15; 34(18):3094-3100). Subsequently, mutations can be derived from the read alignments and their comparison to a reference sequence using variant calling tools, for example Genome Analysis ToolKit (GATK), MuTect, Varscan, and the like (McKenna et al. Genome Res. 2010 September; 20(9):1297-303), which are often used for identification of short insertions and deletions (indels) or single nucleotide variations. Specific software is available for using read alignments for identification of large structural genomic rearrangements, including but not limited to deletions, duplications, inversions, insertions and translocations. An example of such software is GRIDSS, which uses split-read and read-pair mappings and retrieves the sequences of genomic rearrangement breakpoint-junctions through assembly of discordantly mapping sequence reads (Cameron et al. Genome Res 2017 27:2050-2060). Other existing software tools are Delly (Rausch et al. Bioinformatics 2012 28:i333-i339), or Manta (Chen et al. Bioinformatics 2016 32:1220-2), which are based on similar principles. An overview of the methods to identify genomic rearrangements in cancer genomes can be found in the paper by Kosugi et al (Kosugi et al. Genome Biol 2019 20:117). Following the identification of breakpoint-junctions of genomic rearrangements, one can perform an annotation step to identify Frames, i.e. determining the effects of the genomic rearrangement on the protein sequences, using known information on gene structure, transcript sequences, as available in e.g. the Ensembl database (http://www.ensembl.org/index.html). Methods for annotation of indels and genomic rearrangements resulting in frameshift neoORFs and out of frame fusions are (for example) Annovar (Wang et al. Nucleic Acids Res 2010 38:e164) or Integrate-Neo (Zhang et al. Bioinformatics 2017 33:555-557).

A preferred method for identification of neoantigens, in particular Frames resulting from SVs, comprises the in silico reconstruction of rearranged genomic regions and resulting mRNA sequences by using whole genome sequencing, or more preferably a combination of whole genome sequencing and RNA sequencing. In some embodiments the method uses a combination of whole genome sequencing and ribosome profiling and RNA sequencing, or a combination of whole genome sequencing, long-read whole genome sequencing and ribosome profiling and short-read RNA sequencing and long-read RNA sequencing. An approach for analysis of the neoantigens based on such sequencing data, then may involve the following steps, or variations of these steps:
(i) mapping of genome sequencing data of tumor and healthy tissue to a reference human genome sequence, (ii) identification of genomic rearrangement breakpoint junctions from discordantly mapped sequence reads, (iii) assembling full length transcripts from RNA sequence reads that are spanning or in close vicinity to rearrangement breakpoint-junctions, (iv) identification of translation start sites in the assembled transcript sequences, (v) translation of neoORFs present in said assembled transcript sequences to predict associated protein sequences, and (vi) checking that said protein sequences are not present in any known human protein databases, by BLAST searches, or the like.

The identification of neoantigens can be difficult if the identification method only makes used of DNA sequencing, in particular if a new junction is in the mature mRNA is created by a novel splicing event. In many cases it is not possible to predict the neoantigen based solely on the DNA sequence.

For example, Hidden Frames cannot be predicted based solely on DNA sequence using standard methods. The resulting Frame will depend not only on the DNA rearrangement (i.e., structural variation) but also on the splicing machinery. The vast majority of DNA rearrangements occur in non-coding DNA, e.g., in the non-coding region of a gene (e.g., an intron). The sequences immediately surrounding the rearrangement junction will therefore normally not correspond to the splicing junction in the resulting mRNA and will normally not be present in the resulting corresponding mRNA. Similarly, Splicing Frames (resulting from point mutations, indels, or SVs) are also difficult to predict as the resulting Frames also depend on the splicing machinery. In order to address these problems, the methods provided herein comprise both whole genome sequencing as well as long-read RNA sequencing.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using a purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns. Numerous RNA isolation kits are commercially available and can be used in the methods of the invention.

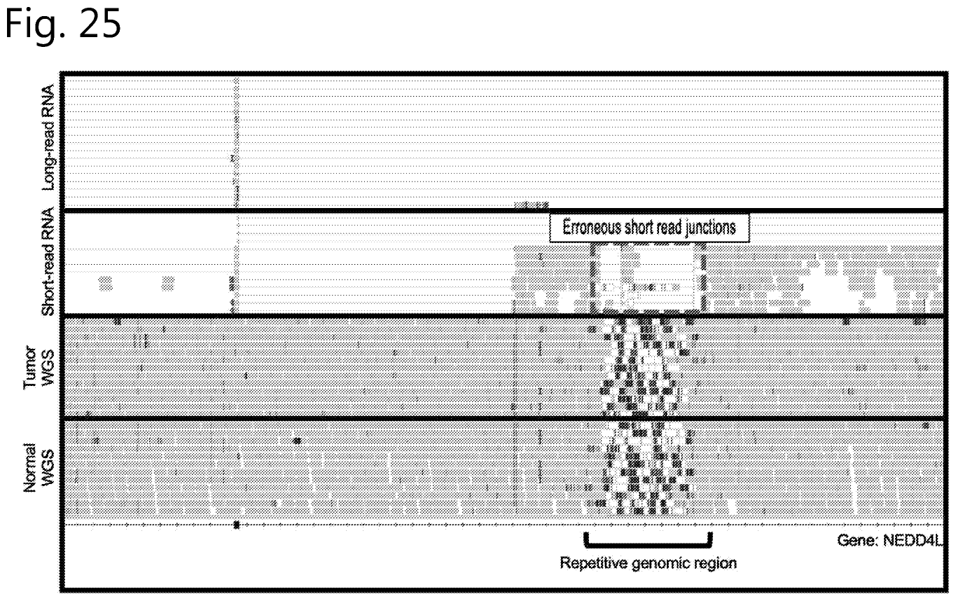

Preferably, the RNA isolated for sequencing is cytosolic RNA that is not tRNA or rRNA. Preferably, the RNA is poly-(A)RNA. Methods for selecting poly-(A) RNA are known to a skilled person and include mixing total RNA with poly-(T) oligomers and retaining only the RNA that is bound to the poly-(T) oligomers. Preferably, the RNA is selected for having a 5′-CAP. More preferably, the RNA is selected for having a 5′-CAP and a 3′-poly-(A) tail (FIG. 25). Preferably the mRNA is poly-(A) RNA having a 5′ CAP. Suitable methods are known to a skilled person. For example, the TeloPrime Full-Length cDNA Amplification Kit V2 from Lexogen uses Cap-Dependent Linker Ligation (CDLL) and long reverse transcription (long RT) technology to select full-length RNA molecules that are both capped and polyadenylated. Other methods include the use of a mRNA 5′ Cap Structure Affinity Column Preparation as described in U.S. Pat. No. 6,187,544B1. Preferably, the methods disclosed herein further comprise a purification step of enriching for or selecting for mRNA that is poly-(A) RNA or having a 5′ CAP. Preferably, the methods disclosed herein further comprise a purification step of enriching for or selecting for mRNA that is poly-(A) RNA having a 5′ CAP.

In some embodiments, the RNA is reversed transcribed to cDNA and the cDNA is sequenced. In some embodiments direct RNA sequencing is performed. “RNA sequencing” and “RNA sequences” as used herein encompass both direct RNA sequencing and cDNA sequences from the corresponding RNA.

While second-generation (or short-read) sequencing provides highly accurate sequence information, in some cases it can be difficult to correctly annotate longer stretches of sequences, in particular when such sequences involve repetitive elements or complex rearrangements. Long-read sequencing has the advantage that longer stretches of nucleic acid can be sequenced. The methods of the disclosure comprise performing long-read RNA sequencing on RNA or long-read sequencing on the corresponding cDNA. Preferably, long-read sequencing methods are also used to determine DNA sequence. Such methods are often referred to as “third generation sequencing” and include systems from Pacific Biosciences and Oxford Nanopore technologies.

Long read sequencing offers the advantage that the structure of the entire mRNA molecule can be determined. Determining the full-length structure of mRNA molecules resulting from the genomic mutations is useful for identifying Frame neopeptide sequences. This is especially useful for complex rearrangements as well as mutations affecting splicing. For example, the splicing pattern of a gene depends on the structure of the primary transcript. Preferably, long read sequencing is used to confirm the splicing events for gene fusions. In regards to Hidden and Splicing Frames, long read sequencing is preferably also used to confirm that a polyadenylated RNA is produced, and to determine possible (cryptic) splicing patterns. Long-read sequencing is also useful to confirm that the mRNA is not subject to extensive non-sense mediated decay. Long read sequencing is preferably also used to confirm the poly-adenylation of RNA products containing stop loss Frames.

Preferably, the long-read molecules that are sequenced are at least 300 nucleotides in length, more preferably at least 500 nucleotides in length, more preferably covering the full-length mRNA molecules for each expressed gene in a tumor sample. To obtain molecules for long read sequencing the RNA is generally not fragmented during isolation and purification. Methods for sequencing long-read RNA molecules are well-known in the art and are disclosed in publications such as Tilgner, H. et al., Proc. Nat'l Acad. Sci., USA, 111(27):9869-9874 (2014), Tseng, E. and Underwood, J., J. Biomol. Techniques., 24 Supplement:545 (2013), Sharon, D., et al., Nature Biotech. 31(10):1009-1014 (2013), Pan. Q., et al., Nature Genetics, 40:1413-1415 (2008), Steijger, T., et al., Nature Methods, 10:1177-1184 (2013) and U.S. Pat. Nos. 8,192,961, 8,501,405 and 8,940,507, all of which are incorporated by reference. Similar methods are useful for long-read whole genome sequencing (see also Logsdon, Nature Reviews Genetics 2020). Preferably, long-read single molecule DNA and/or RNA sequencing technologies are used in the present methods. Such methods can generate reads of at least 1 kb even tens to thousands of kilobases in length. The accuracy of such methods is constantly improving and, as a skilled person will appreciate, if highly accurate long-read sequence data is available, then short-read sequencing is redundant.

To improve the quality of the long RNA sequences, multiple approaches known to the skilled person may be used.

In one approach, the RNA sequencing preferably includes short-read cDNA sequencing, in addition to the long-read RNA/cDNA sequencing. The short-read RNA sequences are used in subsequent analytical steps to remove errors inherent to single-molecule long-read sequencing. In some embodiments, short-read sequencing methods such as sequencing-by-ligation (SBL) and sequencing-by-synthesis (SBS) are used. Generally, such short-read sequencing methods provide read lengths of around 100-200 bases. These methods are also referred to as second-generation sequencing or Next-generation sequencing.

In another approach, the long-read RNA sequencing may include consensus sequencing, i.e. repeated sequencing of the same molecule and determining a consensus sequence from the repeatedly sequenced copies. For example, long-read sequencing on a Pacific Biosciences sequencer enables Circular Consensus Sequencing (CCS), which involves repeated sequencing of the same template DNA molecule (or cDNA molecule). The repeated sequences can be collapsed to generate a highly accurate consensus sequence, which reaches a sequence accuracy competitive with short-read (RNA) sequencing methods. Circular consensus sequencing involves the generation of long sequence reads with (inverted) tandemly repeated copies of the original transcript molecule. Such concatemer reads can be used to generate a high-quality consensus sequence. Examples of such approach are described in e.g. Wenger et al, Nature Biotechnology volume 37, pages 1155-1162 (2019). Generation of high-quality mRNA transcript reads with such approach have been described (see review by Byrne et al, Philos Trans R Soc Lond B Biol Sci. 2019 Nov. 25; 374(1786): 20190097). Similar consensus sequencing approaches from long reads with repeated copies have been described in combination with Nanopore sequencing (Gigascience 2016 Aug. 2; 5(1):34 and Nucleic Acids Res 2021 Jul. 9; 49(12):e70).

An alternative approach for consensus sequencing involves the use of Unique Molecular Identifiers (UMIs) coupled to each unique and original DNA (or mRNA or cDNA) molecule. A library of nucleic acid molecules tagged with UMI sequences is subsequently amplified by PCR or the like, thereby producing copies of each unique molecule in the library. Following deep long-read sequencing of the amplified library with nucleic acid sequences each containing a UMI, the resulting sequence reads can be clustered based on the presence of (near) identical UMI sequence. The clusters of sequences are then collapsed into a single consensus sequence with higher accuracy than each of the individual sequence reads within the cluster. An example of UMI-based long-read consensus sequencing has been described by Karst et al, Nature Methods 18, page 165-169 (2021).

Once highly accurate consensus sequences are obtained, each individual consensus read (which corresponds to a single mRNA or cDNA molecule) can be directly translated.

Methods provided herein preferably comprise determining the sequences of full-length RNA transcripts encoded by nucleic acid sequences comprising (or overlapping with) the somatic mutations (e.g., DNA rearrangements or splicing mutations). As is clear to a skilled person, sequences immediately surrounding the DNA rearrangement junction will normally not be represented in the full-length RNA transcripts.

In an exemplary embodiment, a method, referred to herein as ‘FramePro’ or ‘reconstructed tumor genome mapping’, comprises the generation of a tumor-specific human reference genome, based on somatic and germline structural genome variations identified in a tumor sample, followed by mapping of long cDNA/RNA reads to the tumor-specific reference sequences. The method comprises the following steps:
a) Whole genome sequencing (WGS) of a tumor sample and a healthy sample from the individual as described further herein. Preferably, WGS of the tumor sample includes long-read sequencing.
b) Long-read RNA sequencing of RNA from at least one tumor sample as described further herein. Preferably the RNA is selected or enriched for poly-(A) mRNA and/or 5′-CAP containing mRNA as described further herein.
c) Optionally performing short-read RNA sequencing on RNA from at least one tumor sample as described further herein.
d) Mapping the genomic sequences obtained to a human reference sequence to identify somatic structural genomic variations in the tumor sample as described further herein. In a preferred embodiment the genomic sequences are mapped to a reference human genome sequence (GRCh37, GRCh38, or the like). This step also distinguishes germline genetic variations (identified from the healthy tissues) from tumor-specific genetic variations (identified from the tumor tissue) as discussed herein.
e) Generating in silico a reconstructed tumor-specific reference genome comprising the identified somatic structural genomic variations. As will be understood by the skilled person, it is not necessary to generate a complete tumor-specific reference genome. Rather, contigs which span the structural genomic variations can be generated. Such contigs are generally around 100 kb but can be longer, e.g., 300-400 kb. Longer contigs may be useful in genomic regions which comprise a large number or re-arrangements. The reconstructed tumor-specific reference genome contigs can be generated by any method known to a skilled person. For example, the genomic DNA segments from the reference human genome sequence can be joined based on the information on breakpoint junctions derived from the WGS (e.g., using SV variant calling). Alternatively, the WGS data comprising the SVs may be directly used in an assembly algorithm to generate assembled contigs covering the rearranged segments.
f) Aligning the RNA sequences to the reconstructed tumor-specific reference genome. The cancer tumor often comprises complex rearrangements which complicate that mapping of RNA sequences, in particular as the order and orientation of exonic sequences in the tumor genome may be different than in the human reference genome.

In some embodiments, this step is an iterative process comprising short-read sequencing data and long-read sequencing data to the reconstructed contigs. The short-read data can be used to polish (i.e., correct) the long-read data.
g) Determining the sequences of the full-length RNA transcripts encoded by the structural genomic variations.
h) Determining the predicted amino acid sequences encoded by the full-length transcripts of g) as further described herein.

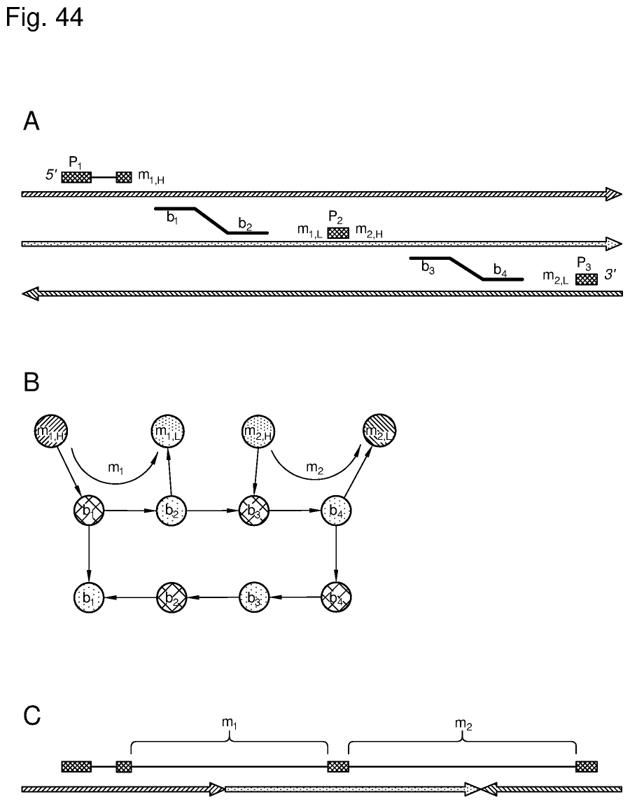

In one embodiment, a method, which we refer to herein as ‘direct-RNA Frame detection’ is provided. Said method comprises the mapping of cDNA/RNA sequencing reads to a normal human reference genome, such as GRCh37, GRCh38 or the like, followed by identification of a possible ‘path’ following genomic rearrangement breakpoint-junctions in the tumor genome that could lead to a contig that places the mapped cDNA/RNA segments together in a small genomic sequence (arbitrarily defined as smaller than e.g. 200 kb). Such method is particularly relevant for identification of Frames emerging from complex genomic rearrangements, such as chromothripsis or the like, which occurs at high-frequency in many human cancers (Cortes-ciriano et al, Nature Genetics volume 52, pages331-341(2020). Complexity of genomic rearrangements may not be fully resolved by short-read WGS or long-read WGS, which makes mapping of long cDNA/RNA reads to the normal human reference a relevant alternative option. The method may involve the following steps or combinations of steps:
a. Long-read RNA or cDNA sequencing of RNA from a tumor sample as described further herein. Preferably the RNA is selected or enriched for poly(A) mRNA and/or 5′ cap containing mRNA as described further herein.
b. Optionally performing short-read RNA or cDNA sequencing on RNA from at least one tumor sample as described further herein.
c. Aligning the RNA/cDNA sequences to the reference genome, such as GRCh37, GRCh38 or alternative human reference genomes. In some embodiments, the short-read RNA data can be used to polish (i.e., correct) the long-read RNA data before alignment to the reference genome.
d. Whole genome sequencing (WGS) of a tumor sample and a healthy sample from the individual as described further herein. Preferably, WGS of the tumor sample includes long-read sequencing, as long-read sequencing may improve the identification and resolving of complex DNA rearrangements (Cretu Stancu et al, Nature Communications 8, 1326 (2017); Nattestad et al, Genome Research 2018 August; 28(8):1126-1135).
e. Mapping the genomic sequences obtained from WGS to a human reference sequence to identify somatic structural genomic variations in the tumor sample as described further herein. In a preferred embodiment the genomic sequences are mapped to a reference human genome sequence (GRCh37, GRCh38, or the like). This step also distinguishes germline genetic variations (identified from the healthy tissues) from tumor-specific genetic variations (identified from the tumor tissue) as discussed herein.
f. In some embodiments, the method comprises identification of a possible linear contig of DNA sequence in the tumor genome sequences that comprises the genomic segments to which the long cDNA/RNA transcript sequence reads are aligned. The order and orientation of said genomic segments should be in agreement with the order and orientation of the exons that are observed in the long transcript read(s) (FIG. 44). The contig may be between 10 kb-1,000 kb, preferably at least 50 kb and on average between 100-300 kb.
g. Generating in silico a reconstructed tumor-specific reference genome comprising the identified genomic segments to which the long-read RNA/cDNA exons align. As will be understood by the skilled person, it is not necessary to generate a complete tumor-specific reference genome. Rather, contigs which span the mapped long-read RNA segments can be generated. Such contigs are generally around 100 kb but can be longer, e.g., 300-400 kb. Longer contigs may be useful if the corresponding transcripts span long distances, e.g. because of large intron sizes. The reconstructed tumor-specific reference genome contigs can be generated by any method known to a skilled person. Preferably, the genomic DNA segments (to which RNA segments align) from the reference human genome sequence can be joined based on the information on breakpoint junctions derived from the WGS (e.g., using structural variant calling). Alternatively, tumor-specific reference contigs can be generated by joining the genomic DNA segments (along with some flanking sequence) to which long-read RNA/cDNA exons align.
h. Aligning the RNA sequences to the reconstructed tumor-specific contigs. In some embodiments, this is a multi-step process comprising mapping short-read RNA/cDNA sequencing data and long-read RNA/cDNA sequencing data to the reconstructed contigs. The short-read RNA data can be used to polish (i.e., correct) the long-read RNA data before the mapping of the long-read RNA/cDNA data and/or after the mapping of the long-read RNA/cDNA data.
i. Determining the sequences of the full-length RNA transcripts encoded by the structural genomic variations. The present disclosure provides that when the transcription/splicing machinery encounters a DNA rearrangement, it will often seek new splice sites resulting in an RNA transcript with a novel open reading frame. Based on the WGS and RNA sequencing data provided above, the sequence of these new RNA transcripts can be determined. In some embodiments, the step involves determining the sequence of the full-length RNA transcripts directly from the (polished) RNA sequencing data. This may be accomplished, e.g., when highly accurate long-read sequence data is available. In some embodiments, this step involves determining the sequence of the full-length RNA transcripts based on the reconstructed tumor-specific reference genome using the information regarding splice junctions obtained from the RNA sequencing data.
j. Determining the predicted amino acid sequences encoded by the full-length transcripts of i) as further described herein.

In preferred embodiments, the method disclosed herein comprises selecting as candidate neoantigen peptide sequences, peptide sequences whose corresponding RNA, preferably poly-(A) and 5′-capped RNA, sequence is present in the tumor sample.

The methods further comprise determining the (predicted) amino acid sequences encoded by the new open reading frames. As is clear to a skilled person, this step may be performed when identifying somatic genomic changes.

In some embodiments, the method comprises defining tumor specific open reading frames by determining strings of one or more consecutive tumor specific amino acids. One or more of the following criteria may be used to consider an amino acid occurring at the relevant position to be tumor specific.
A) An amino acid may be considered tumor specific if the position of the first nucleotide of the triplet encoding the amino acid does not align to a genomic position which is a known wild-type P-site. The term “wild-type P-site” refers to a peptidyl site or the second binding site for tRNA in the ribosome that synthesizes the wild-type protein. A P-site genome may be pre-compiled, e.g., by annotating each position of each reference chromosome as either not overlapping with any known P-site, overlapping a P-site in the sense strand, overlapping a P-site in the antisense strand, or overlapping in both strands. See also Example 7 section 4.5.5 herein.
B) An amino acid may be considered tumor specific if the amino acid is part of at least one k-mer amino acid sequence which does not correspond to a known wild-type human peptide, wherein k is at least 8, preferably 8, 9, 10, or 11. Wild-type human peptide sequences can be extracted from databases known in the art such as ENSEMBL or the RefSeq human database.
C) An amino acid may be considered tumor specific if the amino acid is encoded by a genomic sequence that is downstream of the somatic genomic change, wherein for a cis-splicing mutation each amino acid of said string of one or more consecutive novel amino acids is encoded by a genomic sequence that is downstream of the first novel splice junction.

In some embodiments, criteria A, B, or C may be used to consider an amino acid occurring at the relevant position to be tumor specific. In some embodiments, criteria A and B; B and C; A and C; or A, B, and C may be used to consider an amino acid occurring at the relevant position to be tumor specific.

In order to identify candidate neoantigen peptide sequences with the potential to induce an immune response, neoORFs comprising at least 8, preferably at least 9 contiguous amino acids are selected. A candidate neoantigen peptide sequence preferably comprises at least 8, preferably at least 9 contiguous amino acids encoded by a neoORF. Preferably, the candidate neoantigen peptide sequences comprise at least 15 or at least 20 or at least 25 or more contiguous amino acids encoded by a neoORF. In some embodiments, shorter neoantigen sequences comprising at least 1, 2, 3 or 4 amino acids encoded by a neoORF may also be useful. In those cases, candidate neoantigen peptide sequences comprise additional sequences flanking the neoORF encoded amino acids such that the candidate neoantigen peptide sequences comprise at least 8, preferably at least 9 amino acids (for binding to MHC class I), or up to 25 or more amino acids (for binding to MHC class II). While not wishing to be bound by theory, 8-9 amino acids is considered to be the minimum length of an MHC epitope and peptides having this length are likely to be more amenable to cellular processing and antigen presentation. In some embodiments, candidate neoantigen peptide sequences comprise at least 8 amino acids, wherein at most 7 contiguous amino acids are encoded by the upstream wildtype sequence preceding the tumor-specific neo open reading frame.

In preferred embodiments, the methods further comprise determining whether said neoORFs are expressed in a tumor sample. Expression of neoORFs can be determined by, e.g., determining the presence of the amino acids or peptides encoded by the neoORFs. Methods for determining the sequence of peptides, e.g., using mass spectrometry, are known to a skilled person.

Expression can also be determined by sequencing RNA from at least one tumor sample from the individual. In some embodiments, the sequence of the RNA overlapping the new junctions of DNA sequences resulting from said DNA rearrangements and/or the sequence of the RNA overlapping the mutation is determined. In some embodiments, the entire RNA molecule comprising a neoORF is sequenced.

In some embodiments, neoantigen peptide sequences encoded by RNA sequences that are expressed in the tumor sample at a level of at least 0.1 transcript per million (tpm) are selected. In some embodiments, the transcripts are expressed at a level of at least 1, at least 5, at least 10, or even at a level of at least 100 tpm. TPM represents a relative expression level that is comparable between samples (see, e.g., Zhao et al. 2021 J Translational Medicine 19, 269 (2021). https://doi.org/10.1186/s12967-021-02936-w).

As will be apparent to one of skill in the art, the methods described herein are preferably performed with the aid of a computer. In particular, as is clear to a skilled person, the mapping and/or aligning of such extensive sequencing reads requires the use of computer programs, which are known in the art. In some embodiments, the methods comprise performing whole genome sequencing of a tumor sample to produce at least 100,000, more preferably at least 1,000,000 sequencing reads. In an exemplary embodiment, around 1 billion sequencing reads are produced. In some embodiments, the methods comprise performing long read RNA sequencing to produce at least 10,000, more preferably at least 100,000 sequencing reads. In some embodiments, the methods comprise performing long read RNA sequencing to produce at least 1,000,000, more preferably at least 10,000,000 sequencing reads. In an exemplary embodiment, around 100 million sequencing reads are produced.

As described further herein, the methods described above are particularly useful for identifying the “Framome” of a tumor, which can then be used in the preparation of a vaccine, or other form of immunotherapy, including but not limited to cellular immunotherapy.

The disclosure further provides methods for preparing a vaccine, collection of vaccines, or collection of neoantigens for the immunotherapy-based treatment of cancer in an individual, comprising identifying candidate neoantigen peptide sequences as disclosed herein. Vaccine or collections are prepared comprising peptides having the candidate neoantigen amino acid sequences or comprising nucleic acids encoding said amino acid sequences. Preferably, the vaccine or collection comprises at least 3, at least 4, at least 5, at least 10, at least 15, or at least 20, or at least 50 neoantigens/Frames.

The disclosure further provides methods for preparing antigen or a collection of antigens comprising identifying candidate neoantigen peptide sequences as disclosed herein. The antigens comprise peptides having the candidate neoantigen amino acid sequences or nucleic acids encoding said amino acid sequences. Preferably, the antigen or collection comprises at least 3, at least 4, at least 5, at least 10, at least 15, or at least 20, or at least 50 neoantigens/Frames.

The disclosure provides vaccines, collections of vaccines, and collection of neoantigens for the treatment of cancer obtainable by identifying candidate neoantigens as disclosed herein. The vaccines and collections may comprise peptides having said candidate neoantigen peptide sequences or nucleic acids encoding said peptide sequences. As described herein, said candidate neoantigen peptide sequences may include the entire, or essentially the entire, Framome, or a selection may be made as described herein.

Preferably, vaccines and collections disclosed herein induce an immune response, or rather the neoantigens are immunogenic. Preferably, the neoantigens bind to an antibody or a T-cell receptor. In preferred embodiments, the neoantigens comprise an MHCI or MHCII ligand/epitope.

The major histocompatibility complex (MHC) is a set of cell surface molecules encoded by a large gene family in vertebrates. In humans, MHC is also referred to as human leukocyte antigen (HLA). An MHC molecule displays an antigen and presents it to the immune system of the vertebrate. Antigens (also referred to herein as ‘MHC ligands’) bind MHC molecules via a binding motif specific for the MHC molecule. Such binding motifs have been characterized and can be identified in proteins. See for a review Meydan et al. 2013 BMC Bioinformatics 14:S13.

MHC-class I molecules typically present the antigen to CD8 positive T-cells whereas MHC-class II molecules present the antigen to CD4 positive T-cells. The terms “cellular immune response” and “cellular response” or similar terms refer to an immune response directed to cells characterized by presentation of an antigen with class I or class II MHC involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

In preferred embodiments, the present disclosure involves the stimulation of an anti-tumor CTL response against tumor cells expressing one or more tumor-expressed antigens (i.e., Frames) and preferably presenting such tumor-expressed antigens with class I MHC.

Frames may be analysed by known means in the art in order to identify potential MHC binding peptides (i.e., MHC ligands). Suitable methods are described herein in the examples and include in silico prediction methods (e.g., ANNPRED, BIMAS, EPIMHC, HLABIND, IEDB, KISS, MULTIPRED, NetMHC, PEPVAC, POPI, PREDEP, RANKPEP, SVMHC, SVRMHC, and SYFFPEITHI, see Lundegaard 2010 130:309-318 for a review). MHC binding predictions depend on HLA genotypes, furthermore it is well known in the art that different MHC binding prediction programs predict different MHC affinities for a given epitope. See also Schmidt et al, Cell Reports Medicine, February 2021.

As will be clear to a skilled person, the neoantigen sequences may also be provided as a collection of tiled sequences, wherein such a collection comprises two or more peptides that have an overlapping sequence. Such ‘tiled’ peptides have the advantage that several peptides can be easily synthetically produced, while still covering a large portion of the Frame. In an exemplary embodiment, a collection comprising at least 3, 4, 5, 6, 10, or more tiled peptides each having between 10-50, preferably 12-45, more preferably 15-35 amino acids, is provided. As will be clear to a skilled person, a collection of tiled peptides comprising a candidate neoantigen peptide sequence indicates that when aligning the tiled peptides and removing the overlapping sequences, the resulting tiled peptides provide the amino acid sequence of the candidate sequence, albeit present on separate peptides.

In some embodiments, the entire candidate neoantigen peptide sequence (i.e., Frame) may be provided as the vaccine (e.g., peptide or nucleic acid). Preferred Frames are at least 8, preferably at least 9 amino acids in length, more preferably at least 20 amino acids in length, more preferably at least 30 amino acids, and most preferably at least 50 amino acids in length. While not wishing to be bound by theory, it is believed that neoantigens longer than 10 amino acids can be processed into shorter peptides, e.g., by antigen presenting cells, which then bind to MHC molecules.

In some embodiments, fragments of a Frame can also be presented as the neoantigen. The fragments comprise at least 8 consecutive amino acids of the Frame, preferably at least 10 consecutive amino acids, and more preferably at least 20 consecutive amino acids, and most preferably at least 30 amino acids. In some embodiments, the fragments can be about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, or about 120 amino acids or greater. Preferably, the fragment is between 8-50, between 8-30, or between 10-20 amino acids. As will be understood by the skilled person, fragments greater than about 10 amino acids can be processed to shorter peptides, e.g., by antigen presenting cells. In an exemplary embodiment, a fragment of a neoantigen peptide sequence as identified herein may be selected based on MHC binding prediction.

In some embodiments, the neoantigens (i.e., peptides) are directly linked. Preferably, the neoantigens are linked by peptide bonds, or rather, the neoantigens are present in a single polypeptide. Accordingly, the disclosure provides polypeptides comprising at least two peptides (i.e., neoantigens). In some embodiments, the polypeptide comprises 3, 4, 5, 6, 7, 8, 9, 10 or more peptides (i.e., neoantigens). In an exemplary embodiment, a polypeptide may comprise 10 different neoantigens, each neoantigen having between 10-400 amino acids. Thus, the polypeptide may comprise between 100-4000 amino acids, or more. As is clear to a skilled person, the final length of the polypeptide is determined by the number of neoantigens selected and their respective lengths. A collection may comprise two or more polypeptides comprising the neoantigens which can be used to reduce the size of each of the polypeptides.

In some embodiments, the amino acid sequences of the neoantigens are located directly adjacent to each other in the polypeptide. For example, a nucleic acid molecule may be provided that encodes multiple neoantigens in the same reading frame. In some embodiments, a linker amino acid sequence may be present. Preferably a linker has a length of 1, 2, 3, 4 or 5, or more amino acids. The use of linker may be beneficial, for example for introducing, among others, signal peptides or cleavage sites. In some embodiments at least one, preferably all of the linker amino acid sequences have the amino acid sequence VDD.

As will be appreciated by the skilled person, the peptides and polypeptides disclosed herein may contain additional amino acids, for example at the N- or C-terminus. Such additional amino acids include, e.g., purification or affinity tags or hydrophilic amino acids in order to decrease the hydrophobicity of the peptide. In some embodiments, the neoantigens may comprise amino acids corresponding to the adjacent, wild-type amino acid sequences of the relevant gene, e.g., amino acid sequences located 5′ to the frame shift mutation that results in the neo open reading frame. Preferably, each neoantigen comprises no more than 20, more preferably no more than 10, and most preferably no more than 5 of such wild-type amino acid sequences.

The peptides and polypeptides can be produced by any method known to a skilled person. In some embodiments, the peptides and polypeptide are chemically synthesized. The peptides and polypeptide can also be produced using molecular genetic techniques, such as by inserting a nucleic acid into an expression vector, introducing the expression vector into a host cell, and expressing the peptide. Preferably, such peptides and polypeptide are isolated, or rather, substantially isolated from other polypeptides, cellular components, or impurities. The peptide and polypeptide can be isolated from other (poly)peptides as a result of solid phase protein synthesis, for example. Alternatively, the peptides and polypeptide can be substantially isolated from other proteins after cell lysis from recombinant production (e.g., using HPLC).

The disclosure further provides nucleic acid molecules encoding the peptides and polypeptide disclosed herein. Based on the genetic code, a skilled person can determine the nucleic acid sequences which encode the (poly)peptides disclosed herein. Based on the degeneracy of the genetic code, sixty-four codons may be used to encode twenty amino acids and translation termination signal.

The nucleic acid molecule may comprise deoxyribonucleic acid (DNA), ribonucleic acid (RNA), or the combination thereof. Nucleic acid molecules include genomic DNA, cDNA, mRNA, recombinantly produced and chemically synthesized molecules. Preferably, the nucleic acid molecule is mRNA. The nucleic acid molecule may be single-stranded or double-stranded and linear or covalently circularly closed molecule. Preferably the nucleic acid molecule is isolated. The nucleic acid molecule may be recombinantly produced or chemically synthesized. RNA, e.g., can be prepared by in vitro transcription from a DNA template.

In some embodiments, the nucleic acid molecule is modified. The chemical modification may comprise replacing or substituting an atom of a pyrimidine base with an amine, SH, an alkyl (e.g., methyl, or ethyl), or a halo (e.g., chloro or fluoro). The chemical modification may also comprise modifications of the sugar moiety and/or phosphate backbone. Chemical modification of the phosphate backbone comprising phosphorothioate linkages can increase nuclease resistance and ensure a longer half-life in the cellular environment. Preferably, the nucleic acid molecule is RNA (preferably mRNA) having one or more modifications. In some embodiments, the nucleic acid is RNA and comprises pseudouridine or another modified nucleoside. Preferably, the nucleic acid molecule is not modified or comprises one or more modified nucleosides selected from 1-methylpseudouridine. Preferably, the nucleosides of the nucleic acid molecule are not modified, except for the optional 5′ cap structure.

Modified nucleosides optionally comprise 1-methyl-3-(3-amino-3-carboxypropyl) pseudouridine, 2′-O-methylpseudouridine, 5-methyldihydrouridine, 5-methoxyuridine, 5-methylcytidine, 2′-O-methyuridine, 1-methylpseudouridine, pyridin-4-one ribonucleoside, 5-aza-uridine, 2-thio-5-aza-uridine, 2-thiouridine, 4-thio-pseudouridine, 2-thio-pseudouri-dine, 5-hydroxyuridine, 3-methyluridine, 5-carboxymethyluridine, 1-carboxymethyl-pseudouridine, 5-propynyluridine, 1-propynyl-pseudouridine, 5-taurinomethyluridine, 1-taurinomethylpseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, Zebularine, 5-aza-Zebularine, 5-methyl-Zebularine, 5-aza-2-thio-Zebularine, 2-thio-Zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, 2-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6.N6-dimethyladenosine, 7-methyladenine, 2-methylthio-adenine, 2-methoxy-adenine, inosine, 1-methyl-inosine, Wyosine, Wybutosine, 7-deaza guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2.N2-dimethylguanosine, 8-oxo-guanosine, 7-methyl-8-oxo-guanosine, 1-methyl-6-thioguanosine, N2-methyl-6-thio-guanosine, and N2.N2-dimethyl-6-thio-guanosine.

Particularly preferred modifications and methods for generating nucleic acid molecules are described in US2020030460, which is hereby incorporated by reference, including the modifications described at paragraphs 0072 and 0292 of US2020030460. Such modifications reduce the immunogenicity of RNA. In preferred embodiments, the modified nucleotide is 2′-O-methylpseudouridine, 2′-O-methyluridine, 5-methoxyuridine, 1-methylpseudouridine, N6-methyladenosine, 2-thiouridine, 5-methylcytidine, 5-methyluridine, pseudouridine, or a combination thereof. In a preferred embodiment, mRNA is provided wherein at least a portion of uridine nucleotides are replaced by 1-methylpseudouridine, 2′-O-methyluridine, 2-thiouridine, 5-methyluridine, 5-methoxyuridine, pseudouridine, or a combination thereof. In some embodiments, mRNA is provided wherein at least a portion of cytidine nucleotides are replaced by 5-methylcytidine.

In a preferred embodiment, the nucleic acid molecules are codon optimized. As is known to a skilled person, codon usage bias in different organisms can affect gene expression level. Various computational tools are available to the skilled person in order to optimize codon usage depending on which organism the desired nucleic acid will be expressed. Preferably, the nucleic acid molecules are optimized for expression in mammalian cells, preferably in human cells. Table 2 lists for each acid amino acid (and the stop codon) the most frequently used codon as encountered in the human exome.

TABLE 2
most frequently used codon for each amino acid
and most frequently used stop codon.

| | |
|---|---|
| A | GCC |
| C | TGC |
| D | GAC |
| E | GAG |
| F | TTC |
| G | GGC |
| H | CAC |
| I | ATC |
| K | AAG |
| L | CTG |
| M | ATG |
| N | AAC |
| P | CCC |
| Q | CAG |
| R | CGG |
| S | AGC |
| T | ACC |
| V | GTG |
| W | TGG |
| Y | TAC |
| Stop | TGA |

In preferred embodiments, at least 50%, 60%, 70%, 80%, 90%, or 100% of the amino acids are encoded by a codon corresponding to a codon presented in Table 2.

In some embodiments, the nucleic acid molecule is mRNA, self-amplifying replicon RNA, circular RNA, or viral RNA. Preferably, the nucleic acid molecule is mRNA. The disclosure further provides vectors comprising the nucleic acids molecules disclosed herein. A “vector” is a recombinant nucleic acid construct, such as plasmid, phase genome, virus genome, cosmid, or artificial chromosome, to which another nucleic acid segment may be attached. The term “vector” includes both viral and non-viral means for introducing the nucleic acid into a cell in vitro, ex vivo or in vivo. The disclosure contemplates both DNA and RNA vectors. The disclosure further includes self-replicating RNA with (virus-derived) replicons, including but not limited to mRNA molecules derived from mRNA molecules from alphavirus genomes, such as the Sindbis, Semliki Forest and Venezuelan equine encephalitis viruses.

Vectors, including plasmid vectors, eukaryotic viral vectors and expression vectors are known to the skilled person. Vectors may be used to express a recombinant gene construct in eukaryotic cells depending on the preference and judgment of the skilled practitioner (see, for example, Sambrook et al., Chapter 16). For example, many viral vectors are known in the art including, for example, retroviruses, adeno-associated viruses, and adenoviruses. Other viruses useful for introduction of a gene into a cell include, but are not limited to, adenovirus, arenavirus, herpes virus, mumps virus, poliovirus, Sindbis virus, and vaccinia virus, such as, canary pox virus. The methods for producing replication-deficient viral particles and for manipulating the viral genomes are well known. In preferred embodiments, the vaccine comprises an attenuated or inactivated viral vector comprising a nucleic acid disclosed herein.

Preferred vectors are expression vectors. It is within the purview of a skilled person to prepare suitable expression vectors for expressing the antigens disclosed herein. An “expression vector” is generally a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. Suitable regulatory sequences including enhancers, promoters, translation initiation signals, and polyadenylation signals may be included. Additionally, depending on the host cell chosen and the vector employed, other sequences, such as an origin of replication, additional DNA restriction sites, enhancers, and sequences conferring inducibility of transcription may be incorporated into the expression vector. The expression vectors may also contain a selectable marker gene which facilitates the selection of host cells transformed or transfected. Examples of selectable marker genes are genes encoding a protein such as G418 and hygromycin which confer resistance to certain drugs, β-galactosidase, chloramphenicol acetyltransferase, and firefly luciferase.

The expression vector can also be an RNA element that contains the sequences required to initiate translation in the desired reading frame, and possibly additional elements that are known to stabilize or contribute to replicate the RNA molecules after administration. Therefore, when used herein, the terms DNA and RNA when referring to an isolated nucleic acid encoding a neoantigen peptide should be interpreted as referring to DNA from which the peptide can be transcribed or RNA molecules from which the peptide can be translated.

The nucleic acid molecule according to the present disclosure optionally comprises a 5′ untranslated region (UTR) and/or a 3′ UTR. The nucleic acid molecule may comprise a poly-A tail. A poly-A tail sequence may mostly or entirely be of adenine nucleotides, analogs or derivates thereof. A poly-A tail may be located adjacent to a 3′ UTR. The nucleic acid molecule may comprise a 5′ cap structure. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A 5′ cap may also be an anti-reverse cap analog. Cap species include m⁷GpppG, m⁷Gpppm⁷G, m⁷3′ dGpppG, m₂^{7,O3,}GpppG, m₂^{7,O3,}GppppG, m₂^{7,O2,}GppppG, m⁷Gpppm⁷G, etc. Preferably, the cap structure is a Cap-1, e.g., a m7G(5′)ppp(5′)(2′OMeA)pG cap. Such a cap can be produced using the CleanCap technology from TriLink Biotechnologies. A cap structure may be located adjacent to a 5′ UTR. Preferably, the nucleic acid molecule according to the present disclosure is mRNA comprising a poly-A tail or a 5′ cap structure. Preferably, the nucleic acid molecule according to the present disclosure is mRNA comprising a poly-A tail and a 5′ cap structure.

Also provided for is a host cell comprising a nucleic acid molecule or a vector as disclosed herein. The nucleic acid molecule may be introduced into a cell (prokaryotic or eukaryotic) by standard methods. As used herein, the terms “transformation” and “transfection” are intended to refer to a variety of art recognized techniques to introduce a DNA into a host cell. Such methods include, for example, transfection, including, but not limited to, liposome-polybrene, DEAE dextran-mediated transfection, electroporation, calcium phosphate precipitation, microinjection, or velocity driven microprojectiles (“biolistics”). Such techniques are well known by one skilled in the art. See, Sambrook et al. (1989) Molecular Cloning: A Laboratory Manual (2 ed. Cold Spring Harbor Lab Press, Plainview, N.Y.). Alternatively, one could use a system that delivers the DNA construct in a gene delivery vehicle. The gene delivery vehicle may be viral or chemical. Various viral gene delivery vehicles can be used with the present invention. In general, viral vectors are composed of viral particles derived from naturally occurring viruses. The naturally occurring virus has been genetically modified to be replication defective and does not generate additional infectious viruses, or it may be a virus that is known to be attenuated and does not have unacceptable side effects.

Preferably, the host cell is a mammalian cell, such as MRC5 cells (human cell line derived from lung tissue), HuH7 cells (human liver cell line), CHO-cells (Chinese Hamster Ovary), COS-cells (derived from monkey kidney (African green monkey), Vero-cells (kidney epithelial cells extracted from African green monkey), Hela-cells (human cell line), BHK-cells (baby hamster kidney cells, HEK-cells (Human Embryonic Kidney), NSO-cells (Murine myeloma cell line), C127-cells (nontumorigenic mouse cell line), PerC6®-cells (human cell line, Crucell), and Madin-Darby Canine Kidney (MDCK) cells. In some embodiments, the disclosure comprises an in vitro cell culture of mammalian cells expressing the neoantigens obtained as disclosed herein. Such cultures are useful, for example, in the production of cell-based vaccines, such as viral vectors expressing the neoantigens disclosed herein.

As is clear to a skilled person, if multiple neoantigens are used, they may be provided in a single composition (e.g., a single vaccine composition) or in several different compositions to make up a collection (such as a vaccine collection). The disclosure thus provides collections (such as a vaccine collection) comprising a collection of tiled peptides, collection of peptides, as well as nucleic acid molecules, vectors, or host cells. As is clear to a skilled person, such collections may be administered to an individual simultaneously or consecutively (e.g., on the same day) or they may be administered several days or weeks apart.

Various known methods may be used to administer the vaccines and other therapeutic compounds disclosed herein to an individual in need thereof. For instance, one or more neoantigens can be provided as a nucleic acid molecule directly, as “naked DNA”. Neoantigens can also be expressed by attenuated viral hosts, such as vaccinia or fowlpox. This approach involves the use of a virus as a vector to express nucleotide sequences that encode the neoantigen. Upon introduction into the individual, the recombinant virus expresses the neoantigen peptide, and thereby elicits a host CTL response. Vaccination using viral vectors is well-known to a skilled person and vaccinia vectors and methods useful in immunization protocols are described in, e.g., U.S. Pat. No. 4,722,848. Another vector is BCG (Bacille Calmette Guerin) as described in Stover et al. (Nature 351:456-460 (1991)).

In preferred embodiments, the neoantigens are provided as one or more RNA or DNA vaccines. Such RNA and DNA based vaccines, as well as their preparation, formulation, and therapeutic administration are well-known to a skilled person. See, e.g., U.S. Pat. No. 9,334,328, which is hereby incorporated by reference, which describes pharmaceutical compositions comprising modified nucleosides, nucleotides, and nucleic acids for treating disorders and diseases. The vaccines may also include one or more so-called IRES (“internal ribosomal entry site) An IRES can be used to allow the translation of several peptides or polypeptides independently of one another (“multicistronic” or “polycistronic” mRNA).

Preferably, the vaccine and other therapeutic compositions disclosed herein comprise a pharmaceutically acceptable excipient and/or an adjuvant. The compositions may contain pharmaceutically acceptable auxiliary substances as required to approximate physiological conditions, such as pH adjusting and buffering agents, tonicity adjusting agents, wetting agents and the like. Suitable adjuvants are well-known in the art and include, aluminum (or a salt thereof, e.g., aluminium phosphate and aluminium hydroxide), monophosphoryl lipid A, squalene (e.g., MF59), and cytosine phosphoguanine (CpG). A skilled person is able to determine the appropriate adjuvant, if necessary, and an immune-effective amount thereof. As used herein, an immune-effective amount of adjuvant refers to the amount needed to increase the vaccine's immunogenicity in order to achieve the desired effect.

The disclosure further provides a pharmaceutical composition comprising the nucleic acid molecule as disclosed herein and a lipid-based carrier. Natural lipid-based carriers include cells and cellular membranes. Artificial lipid-based carriers include liposomes, nanoliposomes, micelles, nanoparticles, and lipoplexes. Preferably the lipid-based carrier is selected from lipid nanoparticles, liposomes, lipoplexes, and nanoliposomes. Preferably, the lipid based carrier is a lipid nanoparticle.

In some embodiments, the lipid-based carriers comprise at least one lipid selected from a cationic lipid or ionizable lipid, a neutral lipid or phospholipid, a steroid or steroid analog, an aggregation-reducing lipid, or any combinations thereof. In a preferred embodiment the lipid based carriers comprise i) at least one cationic or cationizable lipid, ii) at least one neutral lipid or phospholipid, iii) at least one steroid or steroid analogue, and iv) at least one aggregation-reducing lipid.

Preferably, the vaccine, peptide antigen, nucleic acid molecule encoding said peptide antigen or collection of vaccines, antigens, and nucleic acid molecules; respectively comprises all of the candidate neoantigen peptide sequences identified in the tumor sample of an individual. Preferably, the vaccine, peptide antigen, nucleic acid molecule encoding said peptide antigen or collection of vaccines, antigens, and nucleic acid molecules; respectively comprises all of the candidate neoantigen peptide sequences identified in the tumor sample of an individual which are also expressed in the tumor (e.g., RNA encoding said neoantigens is present in the tumor). While not wishing to be bound by theory, the use of the full Framome as a vaccine is believed to increase the success rate of the vaccine.

The therapeutic compounds and compositions disclosed herein (e.g., vaccine, peptide antigen, and nucleic acid molecule encoding said peptide antigen) are preferably designed to maximize the number of neoantigen amino acids provided (either as peptides or nucleic acids encoding said peptides) to an individual afflicted with cancer. In some embodiments, the vaccine is an F50 or F100 product, i.e, the vaccine comprises at least 50 or at least 100 neoantigen amino acids encoded in the tumor genome and resulting from neoORFs (Framome), preferably, detected in the RNA of the tumor. In some embodiments, the vaccine is an F200, F500, or F1000 product, i.e, the vaccine comprises at least 200, 500, or 1000, respectively, neoantigen amino acids encoded in the tumor genome and, preferably, detected in the RNA of the tumor. Similarly, in some embodiments, a peptide antigen or a collection of peptide antigens comprises at least 50, at least 100, at least 200, at least 500, or at least 1000 amino acids encoded by the tumor specific open reading frames. The disclosure further provides nucleic acid molecules encoding said antigens.

In some embodiments, there may be reasons to select a subset of the Framome for preparation of a vaccine. For example, if the vaccine is produced as a peptide, or collection of peptides, then a set of between 5-20 peptides preferably having between 20-30 amino acids per peptide may be used. In which case, such an exemplary vaccine would cover a Framome of between 100-500 amino acids.

In some embodiments, the neoantigens are selected based on cysteine content. As known to a skilled person, when the vaccine is a synthetic peptide, or collection of synthetic peptides, the amino acid content may be evaluated to determine whether peptide synthesis and mixing of peptides is possible. Peptide cysteine content is an important factor since cysteines can form disulfide bridges, which may lower solubility and trigger clutting. Frames with the lowest cysteine content are therefore preferred. The simplest method for determining cysteine content is defined as Qcys=N/L, where N is defined as the number of Cysteines in a Frame and L the total length in amino acids of the Frame. However, other methods are considered as well, for example the number of subsequences of a Frame of defined length L, which have a cysteine content (Q) larger than a predefined value, where L∈{5, 6, 7, 8, 9, 10, 11, . . . , n} with n being the entire length of the Frame sequence in amino acids, and Q being the cysteine content of a Frame subsequence defined as above (N/L). In preferred embodiments, the cysteine content for each peptide is 30% or less, more preferably, 5% or less. In preferred embodiments, methods are provided for identifying neoantigen sequences wherein the cysteine content for each peptide is 30% or less, where cysteine content (Qcys) is defined as the number of cysteines in said sequence divided by the total number of amino acids in said sequence.

In some embodiments, “self-peptides” are not included in the neoantigen vaccine or collection. In preferred embodiments, methods are provided for identifying neoantigen sequences wherein the tumor specific open reading frames do not share a contiguous stretch of at least 4 amino acids with human protein reference sequences. Preferably, the candidate neoantigen peptide sequences, or rather the sequence encoded by the tumor specific open reading frames, do not share a contiguous stretch of at least 4, preferably at least 6, amino acids with human protein reference sequences. Such human reference sequences are available at the NCBI RefSeq database. Other protein databases for identifying a matching pattern include, for example uniprot (https://www.uniprot.org/) or proteomics databases (https://www.proteomicsdb.org/).

In some embodiments, candidate neoantigen sequences are selected on the basis of genomic variant allele frequency (VAF), to select clonal (or truncal) neoantigen sequences, i.e. neoantigens present in all tumor cells of a tumor and not in only a subset of the tumor cells. As used herein, VAF is defined as: VAF=Rmut/Rtot where Rmut is the number of sequencing reads in the genome sequencing data containing the frameshift mutation or genomic rearrangement breakpoint junctions, and Rtot is the total number of sequencing reads covering the frameshift mutation locus. A corrected VAF (VAFcor) can be subsequently calculated based on the estimated tumor purity. Preferably, candidate sequences have a VAF or VAFcor of at least 0.1, more preferably >0.1, more preferably >0.2. In preferred embodiments, methods are provided for identifying neoantigen sequences wherein the genomic variant allele frequency of the respective somatic mutation in the tumor cells of a tumor sample is at least 0.1.

In some embodiments, candidate neoantigen sequences are selected which are predicted to comprise an MHC I or MHC II binding epitope, as disclosed further herein. In preferred embodiments, methods are provided for identifying neoantigen sequences wherein the peptides are predicted to comprise one or more MHC I and/or MHC II binding epitopes.

In some embodiments, candidate neoantigen sequences are selected to optimize the physical spread of Frames across the chromosomes. In particular, candidate neoantigen sequences are selected for which the underlying somatic mutations have a maximum distance with regard to chromosomal location. While not wishing to be bound by theory, a single neoORF may be lost, for example via chromosome loss or deletion. However, the chance that two neoORFs located on different chromosomal arms are both lost is highly unlikely. The use of neoORFs distally located from each other is therefore a useful strategy to reduce the risk of antigen loss. The selection of such neoORFs may be useful if the use of the full Framome as a vaccine or other therapeutic composition has practical limitations. In some embodiments, methods are provided for identifying and selecting neoantigen sequences for which the underlying somatic mutations have a maximum distance with regard to chromosomal location, preferably wherein each mutation is separated by at least 20 Mb, at least 50 Mb, or at least 100 Mb In preferred embodiments, methods are provided for identifying and selecting neoantigen sequences for which the underlying somatic mutations have a maximum distance with regard to chromosomal location, preferably wherein each mutation is located on a different chromosomal arm.

There are multiple ways to choose a set of Frames based on their chromosomal locations. One possible approach is as follows. Let d be the number of Frames to be selected. Let F={f₁, f₂, . . . , fₙ} be the set of all Frames within a patient. Let c_{f}_{ᵢ} correspond to the chromosome of frame fᵢ. Let

$$V = ( \begin{matrix}F \\ d\end{matrix} ) = \{ {v}_{1} , {v}_{2} , ... . , {v}_{k} \}$$

be the set of unique subsets of d Frames taken from F. The preferred combination of Frames is

$${v}_{preffered} = {arg max}_{v ∈ V} {\sum }_{a ∈ v} {\sum }_{b ∈ v} ( 1 - {δ}_{{c}_{a} , {c}_{b}} )$$

In some embodiments, neoantigen peptide sequences are selected wherein each somatic mutation corresponding to the neoantigen is located on a different chromosomal arm.

In preferred embodiments, the vaccine, peptide antigen, nucleic acid encoding said peptide antigen or collection of same, respectively; comprises all of the candidate neoantigen peptide sequences identified in the tumor sample of an individual which are also expressed in the tumor (e.g., RNA encoding said neoantigens is present in the tumor) and which are not “self-peptides” as disclosed herein.

In preferred embodiments, the vaccine, peptide antigen, nucleic acid encoding said peptide antigen or collection of same, respectively; comprises all of the candidate neoantigen peptide sequences identified in the tumor sample of an individual which are also expressed in the tumor (e.g., RNA encoding said neoantigens is present in the tumor), which are not “self-peptides” as disclosed herein, and have a VAF or VAFcor of at least 0.1.

In preferred embodiments, the vaccine, peptide antigen, nucleic acid encoding said peptide antigen or collection of same, respectively; comprises all of the candidate neoantigen peptide sequences identified in the tumor sample of an individual which are also expressed in the tumor (e.g., RNA encoding said neoantigens is present in the tumor) and have a VAF or VAFcor of at least 0.1.

In preferred embodiments, the vaccine, peptide antigen, nucleic acid encoding said peptide antigen or collection of same, respectively; comprises all of the candidate neoantigen peptide sequences identified in the tumor sample of an individual which are also expressed in the tumor (e.g., RNA encoding said neoantigens is present in the tumor), which are not “self-peptides” as disclosed herein, have a VAF or VAFcor of at least 0.1, and comprise a predicted MHC I or MHC II binding epitope.

The methods describe determining the presence of cis-splicing mutations that result in tumor specific open reading frames. In some embodiments, the methods further comprise comparing the splice junction resulting from the cis-splicing mutation with a database of mRNA wild-type splice junctions and selecting as candidate neoantigen peptide sequences those sequences where said splice junction is not present in the database of mRNA wild-type splice junctions. Databases comprising human mRNA wild-type splice junctions are known to the skilled person and include the GTex database (see the world wide web at gtexportal.org/home), the RJunBase database (see the world wide web at rjunbase.org), H-DBAS—Human-transcriptome DataBase for Alternative Splicing (see the world wide web at h-invitational.jp/h-dbas/), and the Alternative Splicing Database (ASD) (see Stefan Stamm, et al. ASD: a bioinformatics resource on alternative splicing, Nucleic Acids Research, Volume 34, Issue suppl_1, 1 Jan. 2006, Pages D46-D55, https://doi.org/10.1093/nar/gkj031).

In some embodiments, the disclosure provides neoantigen sequences that are shared by cancer patients. In some embodiments, methods are provides comprising identifying candidate neoantigen sequences from a plurality of individuals. Such neoantigen sequences may be identified from, e.g., newly diagnosed cancer patients or from tumor sequence databases (e.g., TCGA database). Shared neoantigens identified from at least two individuals are selected.

Such shared neoantigens are useful in the treatment of cancer and may be used, e.g., in the treatments disclosed herein. In an exemplary embodiment, one or more shared neoantigens (or treatments based on said shared neoantigens, e.g., one or more nucleic acid molecules encoding one or more shared neoantigens, one or more binding molecules that binds the one or more shared neoantigens, one or more T-cells expressing T-cell receptors or chimeric antigen receptors with specificity for one or more shared neoantigens, etc.) are administered to an individual afflicted with cancer.

The disclosure also provides the use of the neoantigens disclosed herein for the treatment of disease, in particular for the treatment of cancer in an individual. It is within the purview of a skilled person to diagnose an individual with as having cancer.

In a preferred embodiment, the cancer is not Microsatellite instable (MSI), in particular the cancer is not MSI-H (i.e., high amount of microsatellite instability). MSI is due to defects in DNA mismatch repair. MSI screening tests are available which analyse changes in the DNA sequence between normal tissue and tumor tissue and can identify the level of instability. In some embodiments, MSI-H cancer is defined as the presence of mutations in 30% or more of microsatellites. In some embodiments, the case is MSI.

In some embodiments, the cancer is colorectal cancer, lung cancer, stomach cancer, non-small lung cancer, pancreatic cancer (i.e. pancreatic ductal adenocarcinoma), head and neck cancer, colorectal cancer, glioblastoma, triple-negative breast cancer, melanoma, breast adenocarcinoma, or renal cell carcinoma.

As used herein, the terms “treatment,” “treat,” and “treating” refer to reversing, alleviating, or inhibiting the progress of a disease, or reversing, alleviating, delaying the onset of, or inhibiting one or more symptoms thereof. Treatment includes, e.g., slowing the growth of a tumor, reducing the size of a tumor, and/or slowing or preventing tumor metastasis.

Suitable compounds for treatment are as disclosed herein and include neoantigen vaccines, peptide antigens, and nucleic acid molecules encoding said peptide antigens and are referred to herein as “the therapeutic compounds”.

As used herein, administration or administering in the context of treatment or therapy of a subject is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners.

The optimum amount of each neoantigen to be included in the vaccine or other therapeutic composition and the optimum dosing regimen can be determined by one skilled in the art without undue experimentation. The composition may be prepared for injection of the peptide, nucleic acid molecule encoding the peptide, or any other carrier comprising such (such as a virus or liposomes). For example, doses of between 1 and 500 mg 50 μg and 1.5 mg, preferably 125 μg to 500 μg, of peptide or DNA may be given and will depend from the respective peptide or nucleic-acid vaccine. Other methods of administration are known to the skilled person. Preferably, the vaccines and other therapeutic composition may be administered parenterally, e.g., intravenously, subcutaneously, intradermally, intramuscularly, or otherwise.

For therapeutic use, administration may begin at or shortly after the surgical removal of tumors. This can be followed by boosting doses until at least symptoms are substantially abated and for a period thereafter.

In some embodiments, the vaccines and other therapeutic compounds disclosed herein may be provided as a neoadjuvant therapy, e.g., prior to the removal of tumors or prior to treatment with radiation or chemotherapy. Neoadjuvant therapy is intended to reduce the size of the tumor before more radical treatment is used.

The vaccines and other therapeutic compounds are preferably capable of initiating a specific T-cell response. It is within the purview of a skilled person to measure such T-cell responses either in vivo or in vitro, e.g. by analyzing IFN-γ production or tumor killing by T-cells. In therapeutic applications, vaccines and other therapeutic compounds are administered to a patient in an amount sufficient to elicit an effective CTL response to the tumor antigen and to cure or at least partially arrest symptoms and/or complications.

The vaccines and other therapeutic compounds can be administered alone or in combination with other therapeutic agents. The therapeutic agent is for example, a chemotherapeutic agent, radiation, or immunotherapy, including but not limited to checkpoint inhibitors, such as nivolumab, ipilimumab, pembrolizumab, or the like. Any suitable therapeutic treatment for a particular, cancer may be administered.

The term “chemotherapeutic agent” refers to a compound that inhibits or prevents the viability and/or function of cells, and/or causes destruction of cells (cell death), and/or exerts anti-tumor/anti-proliferative effects. The term also includes agents that cause a cytostatic effect only and not a mere cytotoxic effect. Examples of chemotherapeutic agents include, but are not limited to bleomycin, capecitabine, carboplatin, cisplatin, cyclophosphamide, docetaxel, doxorubicin, etoposide, interferon alpha, irinotecan, lansoprazole, levamisole, methotrexate, metoclopramide, mitomycin, omeprazole, ondansetron, paclitaxel, pilocarpine, rituxitnab, tamoxifen, taxol, trastuzumab, vinblastine, and vinorelbine tartrate.

Preferably, the other therapeutic agent is an anti-immunosuppressive/immunostimulatory agent, such as anti-CTLA antibody or anti-PD-1 or anti-PD-L1. Blockade of CTLA-4 or PD-L1 by antibodies can enhance the immune response to cancerous cells. In particular, CTLA-4 blockade has been shown effective when following a vaccination protocol.

As is understood by a skilled person the vaccine or other therapeutic compounds as disclosed herein and other therapeutic agents may be provided simultaneously, separately, or sequentially. In some embodiments, the vaccine may be provided several days or several weeks prior to or following treatment with one or more other therapeutic agents. The combination therapy may result in an additive or synergistic therapeutic effect.

The compounds and compositions disclosed herein are useful as therapy and in therapeutic treatments and may thus be useful as medicaments and used in a method of preparing a medicament.

In some embodiments, the disclosure provides methods for the preparation of a cellular immunotherapy, such as personalized neoantigen-specific T-cell therapy. Such cellular immunotherapy is directed against the tumor cells with expressed Frames where Frame-derived peptides are presented in complexes with HLA molecules on the cell surface.

Various methods for the use of neoantigen-specific T-cells or neoantigen-specific T-cell receptors in cancer immunotherapy have been described. T-cell receptors (TCRs) are expressed on the surface of T-cells and consist of an α chain and a β chain. TCRs recognize antigens bound to MHC molecules expressed on the surface of antigen-presenting cells. The T-cell receptor (TCR) is a heterodimeric protein, in the majority of cases (95%) consisting of a variable alpha (α) and beta (β) chain, and is expressed on the plasma membrane of T-cells. The TCR is subdivided in three domains: an extracellular domain, a transmembrane domain and a short intracellular domain. The extracellular domain of both α and β chains have an immunoglobulin-like structure, containing a variable and a constant region. The variable region recognizes processed peptides, among which neoantigens, presented by major histocompatibility complex (MHC) molecules, and is highly variable. The intracellular domain of the TCR is very short, and needs to interact with CD3ζ to allow for signal propagation upon ligation of the extracellular domain.

The major histocompatibility complex (MHC) is a set of cell surface molecules encoded by a large gene family in vertebrates. In humans, MHC is also referred to as human leukocyte antigen (HLA). An MHC molecule displays an antigen and presents it to the immune system of the vertebrate. Antigens (also referred to herein as ‘MHC ligands’) bind MHC molecules via a binding motif specific for the MHC molecule. Such binding motifs have been characterized and can be identified in proteins. See for a review Meydan et al. 2013 BMC Bioinformatics 14:S13.

MHC-class I molecules typically present the antigen to CD8 positive T-cells whereas MHC-class II molecules present the antigen to CD4 positive T-cells. The terms “cellular immune response” and “cellular response” or similar terms refer to an immune response directed to cells characterized by presentation of an antigen with class I or class II MHC involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

With the focus of cancer treatment shifted towards more targeted therapies, among which immunotherapy, the potential of therapeutic application of tumor-directed T-cells is increasingly explored. Such strategies involve the analysis of T-cell receptors (TCRs), either based on T-cells obtained from a tumor specimen, or based on peripheral T-cells from a cancer patient. In vitro characterization of TCRs present on T cells found in tumor specimens or peripheral blood, for their specificity against specific Frame neoantigens could be used to select specific TCR sequences that can be used for development of immunotherapy. Such TCR sequences can, for example, be used for development of TCR-like antibodies (Støkken Høydahl et al, Antibodies 2019, 8, 32). Identified and isolated TCR sequences can also be used for engineering of T-cells, so as to provide them with a specific TCR that recognizes a neoantigen. Several methods for T-cell engineering have been described in the art, including methods to improve the function of T-cells with regard to safety, tumor infiltration and immune stimulation (Rath et al, Cells 2020, 9, 1485).

The disclosure provides methods comprising contacting T-cells with HLA molecules, preferably MHC-I, bound to one or more of the candidate neoantigen peptide sequences identified from an individual according to the methods described herein. In particular, such methods for identifying neoantigens combine whole genome sequencing with long-read RNA/cDNA sequencing to identify neoantigen sequences. The neoantigen peptides used as “bait” are preferably selected based on the potential to bind MHC. Suitable methods to predict MHC binding include in silico prediction methods (e.g., ANNPRED, BIMAS, EPIMHC, HLABIND, IEDB, KISS, MULTIPRED, NetMHC, PEPVAC, POPI, PREDEP, RANKPEP, SVMHC, SVRMHC, and SYFFPEITHI, see Lundegaard 2010 130:309-318 for a review). In some embodiments, T-cells are contacted with neoantigen peptide sequences. The peptide sequences may be provided bound to HLA molecules. In some embodiments, antigen-presenting cells (such as dendritic cells) are transfected with one or more nucleic acid molecules encoding one or more candidate neoantigen peptide sequences and T-cells are contacted with said APCs. The T-cells as well as the mixture of T-cells and APCs can be further cultured and used as an immunotherapy.

In some embodiments, a method is provided that comprises the (i) isolation of T-cells from a tumor specimen (e.g. tumor-infiltrating lymphocytes), peripheral blood, bone marrow, lymph node tissue, or spleen tissue from an individual afflicted with cancer, (ii) identification of Frame neoantigens using methods as described herein, (iii) prediction of MHC class I binding epitopes within the Frame neoantigens sequences, (iv) preparation of Frame peptide—MHC (pMHC) multimers, (v) selection of T-cells using the pMHC molecules. Preferably, the method further comprises the (vi) expansion of selected T-cells using appropriate culture conditions. More preferable the method comprises the infusion of the selected or expanded T-cells back into the patient. In a further exemplary embodiment, neoantigen sequences from an individual are identified as described herein. The neoantigen sequences are screened against a library of TCRs for binding. TCRs identified as positive binders are transfected into the T-cells of said individual and transfected back into said individual.

Methods for the selection and identification of immune cells, preferably T-cells or T-cell receptors with specificity for neoantigens are well-known in the art (see e.g. reviews by Bianchi et al, Front Immunol. 2020; 11: 1215 and Zhao and Cao, Frontiers in Immunology, 2019, https://doi.org/10.3389/fimmu.2019.02250, as well as US20180000913, which is hereby incorporated by reference). For example, predicted MHC-I binding epitopes from the Frame neoantigens are bound to synthetic tetrameric forms of fluorescently labelled MHC Class I molecules. CD8+ T-cells with the appropriate T cell receptor will bind to the labelled tetramers and can be selected by flow cytometry. Other suitable methods include those described in U.S. Pat. No. 7,125,964. Briefly, recombinantly produced biotinylated MHC molecules are attached to avidin coated magnetic beads. Peptides and T-cells are added to the beads. T-cells absorbed to the beads (via the interaction with a peptide-MHC complex) are selected.

In some embodiments, the disclosure provides methods which are not a treatment of the human or animal body and/or methods that do not comprise a process for modifying the germ line genetic identity of a human being.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The invention is further explained in the following examples. These examples do not limit the scope of the invention, but merely serve to clarify the invention.

### EXAMPLES

### Example 1: Correction of Splice Junctions of Long Transcript Reads Based on Short-Read RNA

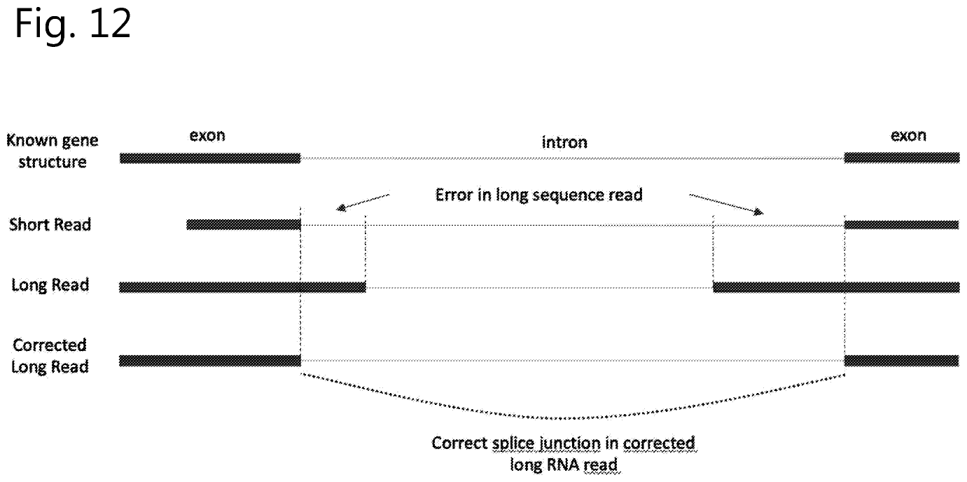

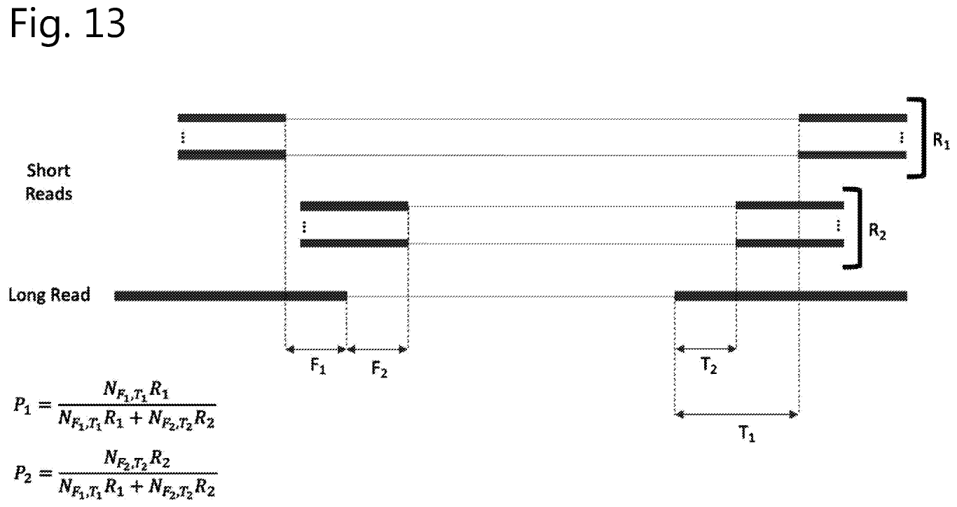

Long read transcript sequences derived from long read single molecule sequencing instruments, such as Oxford Nanopore, have a considerable sequence error rate in the order of 1-10% for molecules that have only been read once. Therefore, identification of splice junctions in long read transcripts is inherently error-prone. To correctly identify splice junctions from long read transcript sequences, we propose to use short read transcript sequencing data (FIG. 12). To improve such correction compared to methods described in prior work (e.g. Broseus et al, Bioinformatics, Volume 36, Issue 20, 15 Oct. 2020), we developed improved methodology as outlined herein (FIG. 13).

Splice junctions in long transcript sequencing reads were corrected using short transcript read junctions for which both the 5′ and 3′ splice sites were within a 15 bp window of the respective long read 5′ and 3′ splice sites. For cases in which multiple different short read splice junctions satisfied this criterion for a given long read junction, the most likely short read junction was chosen via a Bayesian model in which the posterior probability that an observed long read junction arose from an mRNA with a given short read junction was calculated according to:

$$P ( {s}_{i} \vert {F}_{i} , {T}_{i} ) = \frac{P ( {F}_{i} , {T}_{i} \vert {s}_{i} ) P ( {s}_{i} )}{P ( {F}_{i} , {T}_{i} )}$$

Where the event sᵢ is the long read arising from the splice junction i, and the event Fᵢ, Tᵢ is the observation of a long read having a given 5′/3′ distance pair from its underlying original splice sites. The prior probability that a long read arose from an mRNA with splice junction i was calculated according to:

$$P ( {s}_{i} ) = \frac{{R}_{i}}{R}$$

where Rᵢ is the number of short reads supporting junction i and R is the total spliced short reads within the long read splice site window. The probability of observing the splice offset pair Fᵢ, Tᵢ given the long read arose from an mRNA molecule with splice junction i was calculated according to:

$$P ( {F}_{i} , {T}_{i} \vert {s}_{i} ) = \frac{{N}_{{F}_{i} {T}_{i}}}{N}$$

where N_{F}_{ᵢ}_{T}_{ᵢ} is the number of times the given offset pair occurred in all other long read splice junction corrections which were unambiguous because a single short read junction was present within the correction window and N is the total number of unambiguously corrected junctions. The total probability of observing the long-read offset pair Fᵢ, Tᵢ irrespective of any given short read junction can be calculated according to:

$$P ( {F}_{i} , {T}_{i} ) = {\sum }_{j = 1}^{n} P ( {F}_{i} , {T}_{i} \vert {s}_{j} ) P ( {s}_{j} )$$

Where the summation is taken over the n splice junctions within the long-read junction window. Combining these expressions gives:

$$P ( {F}_{i} , {T}_{i} ) = \frac{{N}_{{F}_{i} {T}_{i}} {R}_{i}}{{\sum }_{j = 1}^{n} {N}_{{F}_{j} {T}_{j}} {R}_{j}}$$

Short read splice junctions with the highest probability were chosen to correct long read junctions. Long read splice junctions for which no short read junctions had a correction probability of at least 0.9 were considered uncorrected. Reads which had one or more uncorrected junctions were not considered further.

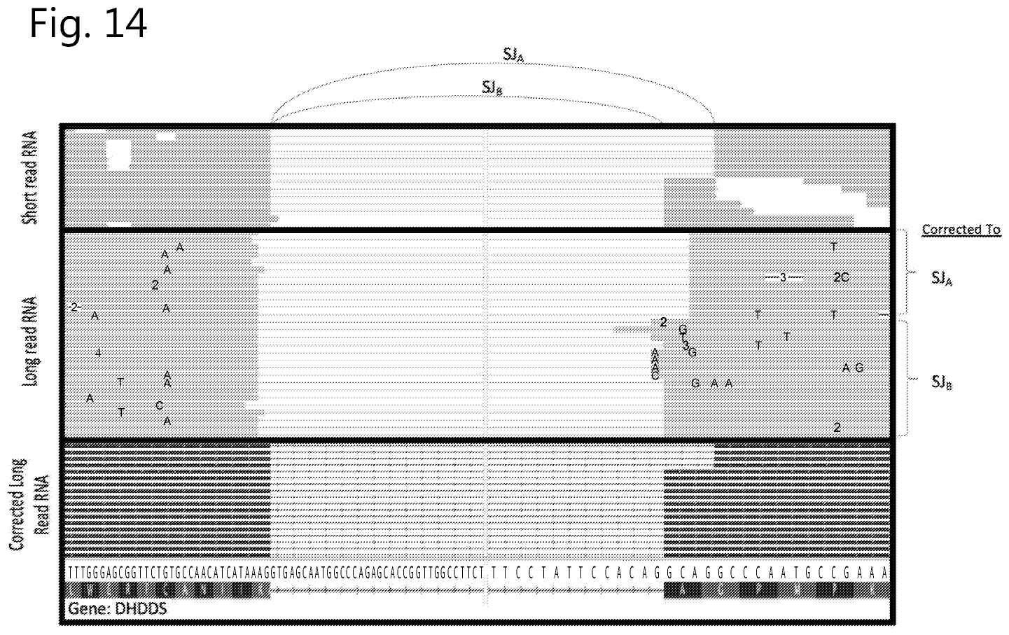

The above Bayesian model was evaluated using long-read and short-read transcriptome sequencing data of a lung cancer. The uncorrected and corrected long transcript reads are depicted in FIG. 14.

### Example 2: Identification of Expressed Splice Frames Resulting from Splice Donor and Acceptor Mutation (LOS)

We have sequenced the genomes of multiple lung tumors using short-read (2*150 bp) whole genome sequencing to a coverage depth of 100× on Illumina HiSeq. Similarly, the corresponding germline genomes were also sequenced to a coverage depth of 30×. The transcriptomes of the lung tumors were sequenced using short-read RNA-sequencing, following the preparation of a cDNA library using the Roche Kappa mRNA prep kit. The cDNA libraries were sequenced on Illumina HiSeq generating approximately 100M paired reads (2*150 bp) per tumor. In addition, we prepared the total RNA of each tumor for long-read sequencing by first performing selection of polyadenylated mRNA molecules using oligo-dT probes and subsequent generation of Capped mRNAs using the TeloPrime procedure, which generates double-stranded cDNA only for mRNA molecules with a 5′ Cap structure. Around 200 ng of polyadenylated and capped mRNA was used for preparation of an Oxford Nanopore sequencing library using kit SQK-LSK109. Between 10Gb to 100Gb of data (˜10M to 100M reads) were generated per tumor sample on a Nanopore GridION or PromethION sequencer.

All classes of genetic variations were called in the short-read whole genome sequencing data using an existing pipeline for read mapping to reference genome GRCh37 and variant calling: https://github.com/hartwigmedical/. From the somatic genomic variant calls, we extracted SNVs that are within 20 bp from a known splice donor or splice acceptor site annotated in the Ensembl database (www.ensembl.org).

Short RNA reads were mapped to the human reference genome GRCh37 using STAR (version 2.7.3a; Dobin et al, Bioinformatics, Volume 29, Issue 1, January 2013, Pages 15-21). Long RNA reads were mapped to human reference genome GRCh37 using minimap2 (version 2.17; Li, Bioinformatics, Volume 34, Issue 18, 15 Sep. 2018, Pages 3094-3100).

The alignment file (BAM) of the long-read RNA sequencing data was used together with the short-read splice junctions to correct the long-read RNA splice junctions, as described in example 1.

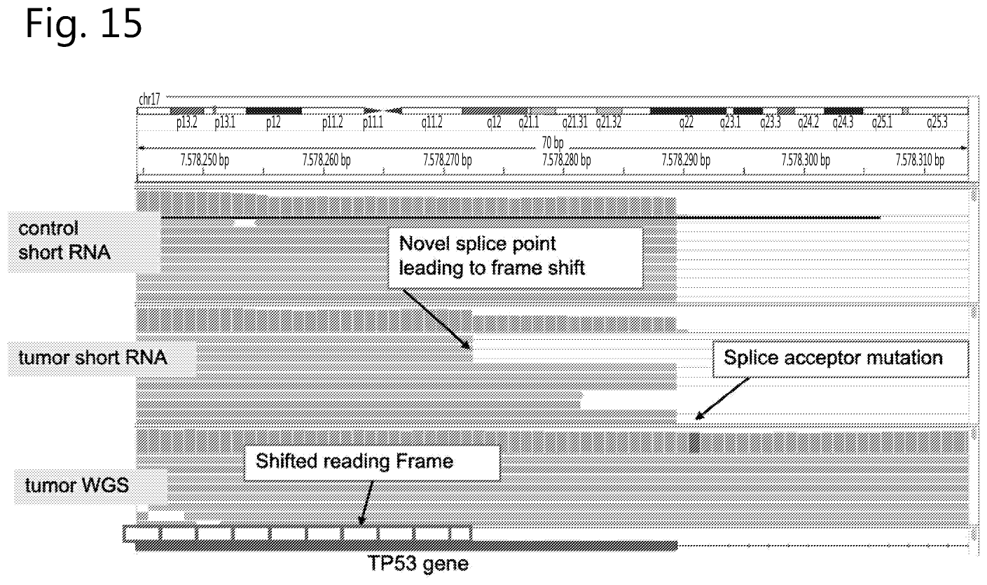

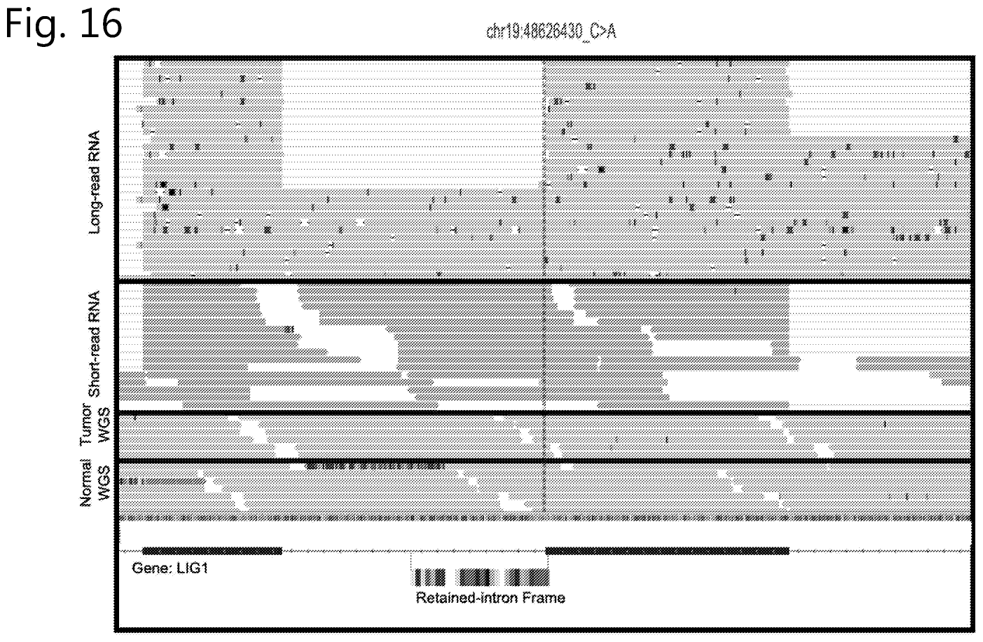

For each aligned and corrected long transcript read (Nanopore) bridging a splice-site mutation (as defined above), the (corrected) splice-junctions were examined and splice-junctions within the effect zone (i.e. between the exon before the splice mutation and the exon after the splice mutation) were checked for uniqueness with respect to known slice-junctions from Ensembl and GTEx (https://gtexportal.org/home/publicationsPage). A threshold for uniqueness with respect to GTEx was defined as a maximum of 10 samples containing the exact splice junction. Unique splice junctions (as defined above and with respect to GTEx), were furthermore required to have support in both short-read RNA and long-read RNA data. Transcript reads containing unique splice junctions according to these criteria were in silico translated by inferring the translation start site based on the overlap of the transcript read with known Ensembl transcripts and their translation start annotations. Translation was performed based on the reference genome sequence, ignoring germline genetic polymorphisms and somatic SVNs. The C-terminal novel part of the in silico predicted protein sequence that is extending beyond the known N-terminal part of the protein is regarded as the Splice Frame sequence. For one lung tumor, a splice site mutation was identified in TP53 gene, affecting a known splice acceptor site (FIG. 15). This splice acceptor mutation leads to a shift in the splice junction of an exon of TP53, causing a shift in the reading frame of the TP53 gene. In an additional lung cancer, a splice donor mutation was observed leading to retention of an intron and an expressed splice Frame (FIG. 16).

### Example 3: Identification of Expressed Splice Frames Resulting from Splice-Site Creating Mutations (GOS)

We have sequenced the genomes lung tumors using short-read (2*150 bp) whole genome sequencing to a coverage depth of 100× on Illumina HiSeq. Similarly, the corresponding germline genomes were also sequenced to a coverage depth of 30×. The transcriptomes of the lung tumors were sequenced using short-read RNA-sequencing, following the preparation of a cDNA library using the Roche Kappa mRNA prep kit. The cDNA libraries were sequenced on Illumina HiSeq generating approximately 100M paired reads (2*150 bp). In addition, we prepared the total RNA for long-read sequencing by first performing selection of polyadenylated mRNA molecules using oligo-dT probes and subsequent generation of Capped mRNAs using the TeloPrime procedure, which generates double-stranded cDNA only for mRNA molecules with a 5′ Cap structure. Around 200 ng of polyadenylated and capped mRNA was used for preparation of Oxford Nanopore sequencing libraries using kit SQK-LSK109. Approximately 68Gb of data (60M reads) were generated on a Nanopore MinION sequencer.

All classes of genetic variations were called in the short-read whole genome sequencing data using an existing pipeline for read mapping to the reference genome GRCh37 and variant calling: https://github.com/hartwigmedical/. From the somatic genomic variant calls, we extracted SNVs that are within a gene, but distant from known splice donor and splice acceptor sites, i.e. further than 20 bp away from a known splice donor or splice acceptor site annotated in the Ensembl database (www.ensembl.org).

Short RNA reads were mapped to the human reference genome GRCh37 using STAR (version 2.7.3a; Dobin et al, Bioinformatics, Volume 29, Issue 1, January 2013, Pages 15-21). Long RNA reads (Nanopore) were mapped to human reference genome GRCh37 using minimap2 (Li, Bioinformatics, Volume 34, Issue 18, 15 Sep. 2018, Pages 3094-3100). The alignment file (BAM) of the long-read RNA sequencing data was used together with the short-read splice junctions to correct the long-read RNA splice junctions, as described in example 1.

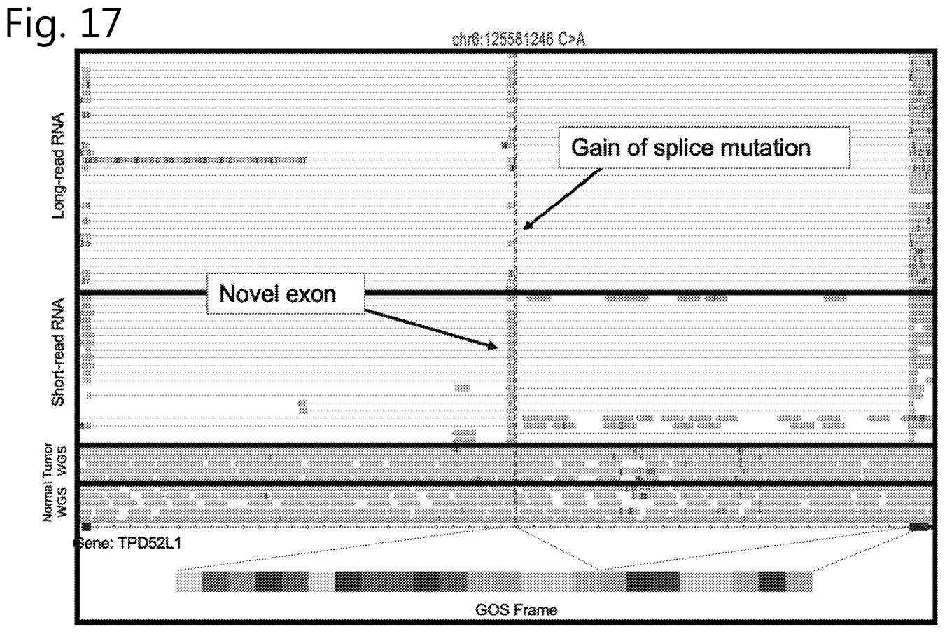

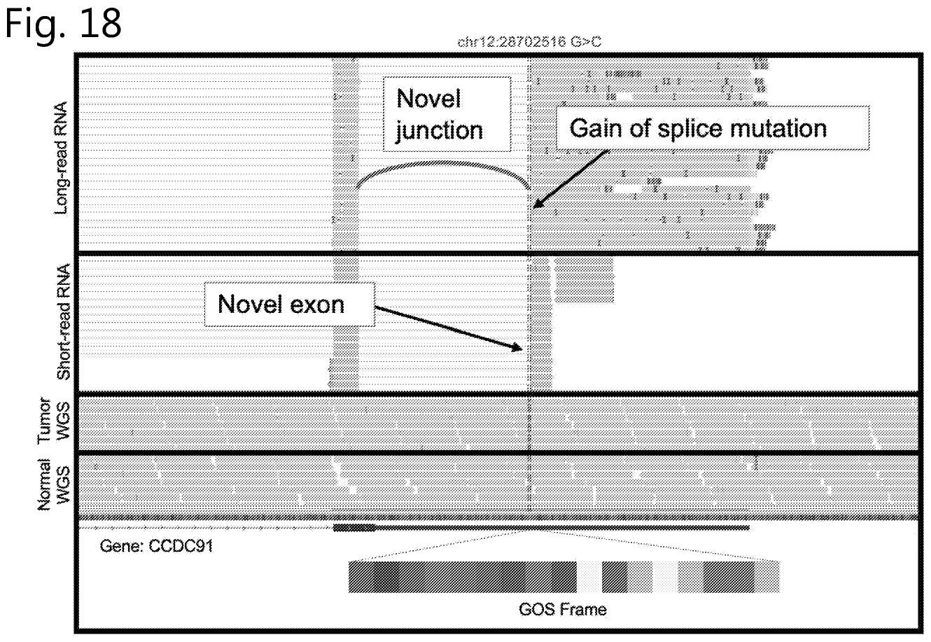

For each aligned and corrected long transcript read (Nanopore) bridging a somatic SNV (as defined above), the (corrected) splice-junctions were examined and splice-junctions within 20 bp from a somatic SNV were checked for uniqueness with respect to known splice-junctions from Ensembl and GTEx (https://gtexportal.org/home/publicationsPage). A threshold for uniqueness with respect to GTEx was defined as a maximum of 10 samples containing the exact splice junction. Unique splice junctions (as defined above and with respect to GTEx), were furthermore required to have support in both short-read RNA and long-read RNA data. Transcripts containing unique splice junctions according to these criteria were in silico translated by inferring the translation start site based on the overlap of the transcript read with known Ensembl transcripts and their translation start annotations. The C-terminal novel part of the in silico predicted protein sequence that is extending beyond the known N-terminal part of the protein is regarded as the Splice Frame sequence (FIG. 17, FIG. 18). Two examples of GOS splice Frames are depicted in FIG. 17 and FIG. 18. In both cases, a novel exon is observed as a result of a splice-site introducing mutation. The novel exon gives rise to a new open reading frame encoding the splice Frame neoantigen.

### Example 4: Identification of Expressed Splice Frames Resulting from Splicing-Affecting Intra-Genic Structural Variants

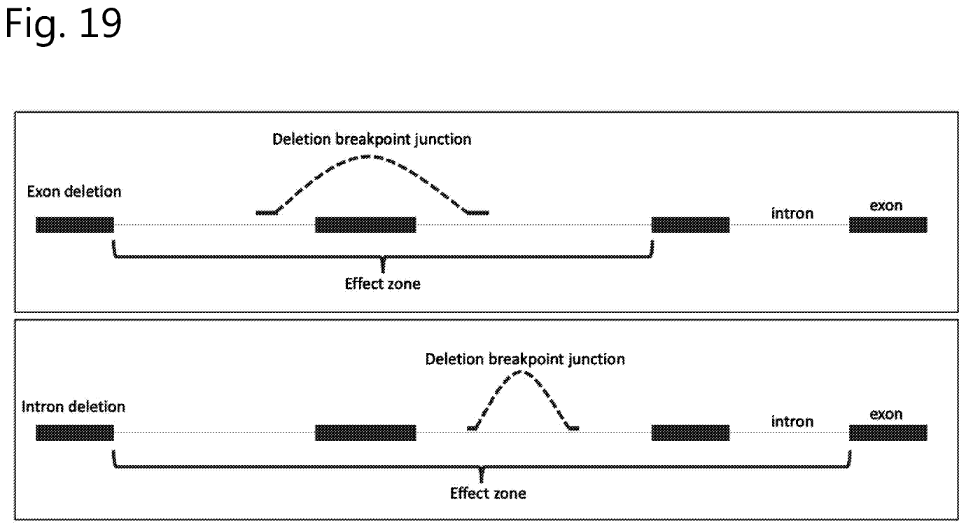

In prior work (WO2021/172990), methodology was described to identify tumor-specific expressed open reading Frame sequences caused by structural genomic variants (SVs). Here, we extend this methodology to identify SVs, primarily deletions, that alter the splicing pattern of mRNAs by creating or deleting splice acceptor or splice donor sites. We sequenced the genomes and transcriptomes of lung tumors as described in Examples 2 and 3. All classes of genetic variations were called in the short-read whole genome sequencing data using an existing pipeline for read mapping to the reference genome GRCh37 and variant calling: https://github.com/hartwigmedical/. From the somatic genomic variant calls, we extracted deletions (larger than 20 bp) for which both breakpoints are within a single gene. Smaller deletions or other types of genetic changes, such as insertions and inversions and duplications are regarded as short indels and are treated as somatic single nucleotide variants, as described in Examples 2 and 3. We discriminated between deletions that encompass exonic sequences and intron/exon boundaries, and deletions that encompass solely intronic sequences and are further than 20 bp away from a known splice junction (FIG. 19).

In a second step, a local in silico reconstruction of the tumor genome was generated based on the identified deletion breakpoint junctions within the gene (FIG. 10). In essence, new tumor-specific reference contigs were created by rearranging segments of the GRCh37 reference genome sequencing according to the orientations and positions of the deletion breakpoint junctions. Contigs size was typically limited to the size of the gene with 100 kb flanking sequences on either side. Flanking sequences were also constructed based on information of somatic SV breakpoint junctions identified in the tumor genome sequencing data. Alternatively, tumor-specific references were constructed based on an RNA-guided approach as described in WO2021/172990. Such RNA-guided approach may be preferred in scenarios where the gene structure is disturbed as a result of (multiple) complex chromosomal rearrangements.

Short RNA reads were mapped to a GRCh37 human reference genome appended with the reconstructed tumor-specific contigs using STAR (version 2.7.3a; Dobin et al, Bioinformatics, Volume 29, Issue 1, January 2013, Pages 15-21). Long RNA reads (Nanopore) were mapped to the same extended reference genome using minimap2 (version 2.17; Li, Bioinformatics, Volume 34, Issue 18, 15 Sep. 2018, Pages 3094-3100). The alignment file (BAM) of the long-read RNA sequencing data was used together with the short-read splice junctions to correct the long-read RNA splice junctions as described in Example 1.

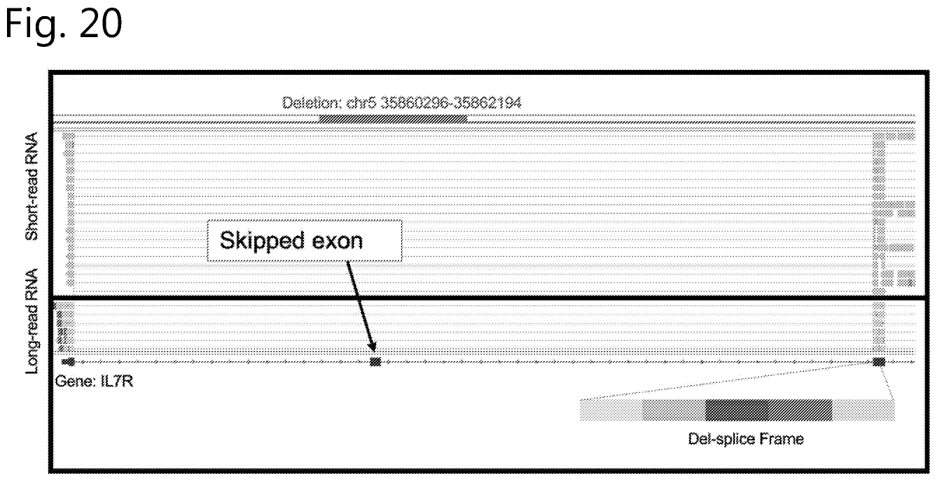

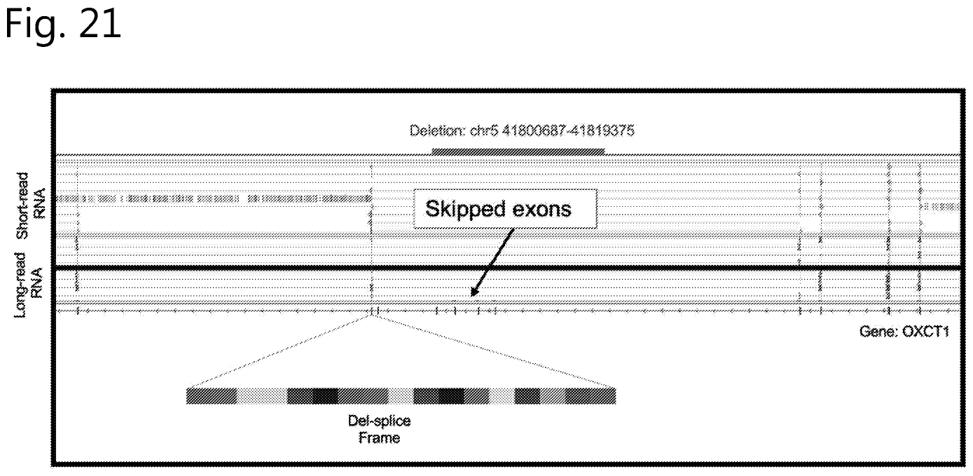

For each aligned and corrected long transcript read (Nanopore) aligning to the rearranged gene and bridging the deletion breakpoint junction, each of the (corrected) splice-junctions was examined. Splice-junctions were checked for uniqueness with respect to known splice-junctions from Ensembl and GTEx (https://gtexportal.org/home/publicationsPage). A threshold for uniqueness with respect to GTEx was defined as a maximum of 10 samples containing the exact splice junction. Unique splice junctions (as defined above and with respect to GTEx), were furthermore required to have support in both short-read RNA and long-read RNA data. Transcripts containing unique splice junctions according to these criteria were in silico translated by inferring the translation start site based on the overlap of the transcript read with known Ensembl transcripts and their translation start annotations. The C-terminal novel part of the in silico predicted protein sequence that is extending beyond the known N-terminal part of the protein is regarded as the Splice Frame sequence. Two examples of tumor-specific intragenic deletions identified in lung tumors and affecting splicing are depicted in FIG. 20 and FIG. 21. In both cases coding exons are deleted leading to novel exon-exon splice junctions and a concomitant novel open reading frame that encodes splice Frame neoantigens.

### Example 5: Contribution of Splice-Frames to the Framomes of Tumours

The presence of expressed Splice Frames was determined in 14 advanced tumors. In addition, we determined other categories of Frames, previously described in WO2021/172990. Tumor samples were analyzed using a combination of multiple sequencing technologies. Genomic DNA was extracted from the tumor sample and the corresponding blood cells of the same patient, using established procedures (Macherey Nagel NuceoSpin or Qiagen DNeasy spin columns). DNA was used for whole genome paired-end sequencing (2×150 bp reads) on Illumina NovaSeq instruments to an average coverage depth of 100× for the tumor sample and 30× for the corresponding blood (control) sample.

In addition, total RNA was isolated from the tumor sample using Macherey Nagel NucleoSpin RNA extraction methods. Total RNA was used for short-read RNA sequencing on Illumina NovaSeq, following ribosomal RNA depletion of total RNA and preparation of a short-read RNA sequencing library from the ribosomal RNA depleted RNA using Illumina TruSeq protocols. Approximately 50 million short paired-end RNA sequencing reads were generated per tumor sample.

Long-read full-length cDNA sequencing was performed using Oxford Nanopore GridION or PromethION technology. Full-length mRNA molecules were selected from total RNA preparations obtained from tumor cells based on the presence of a 5′ CAP and a 3′ poly-A tail. Double-stranded cDNA was prepared from said full length mRNA molecules and the cDNA was sequenced on Oxford Nanopore GridION or PromethION using standard procedures known to skilled persons in the art. At least 10 million full-length transcripts sequences were generated for each tumor sample.

Whole genome sequencing data were analysed using existing bioinformatics methods to identify somatic genetic changes (e.g. as described by Priestley et al, Nature 575, pages 210-216, 2019 and https://github.com/hartwigmedical/), typically resulting in a few thousand somatic point mutations (single nucleotide variations), a few hundred somatic small insertions and deletions (indels), and up to a few hundred of somatic genomic rearrangements (structural variations, SVs), per tumor sample.

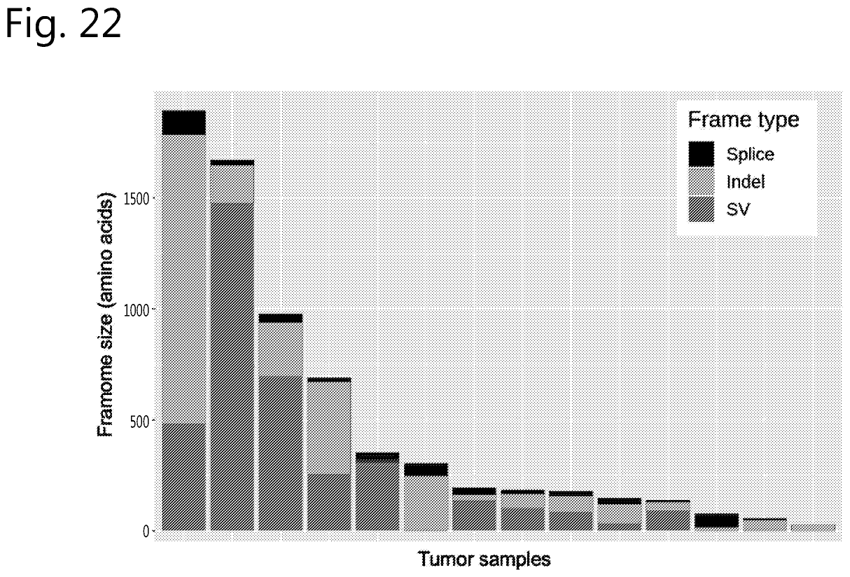

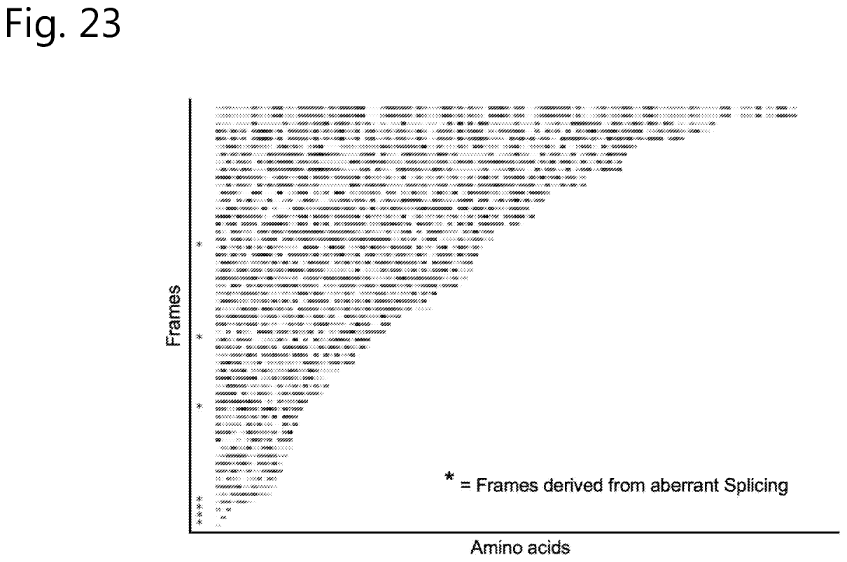

Long-read cDNA (transcript) sequence reads were mapped to the human reference genome (GRCh37) appended with tumor-specific contigs, as determined based on the detected somatic genomic rearrangement breakpoint-junctions in the tumor genome (FIG. 10), and as described herein. Transcript reads were identified that contain novel splice junctions in the vicinity of somatic mutations that were classified as gain-of-splice (GOS), loss-of-splice (LOS) and intragenic deletions, as described in Examples 2, 3 and 4. For genes containing a combination of novel transcript splice junctions (or retained introns) in the vicinity of said somatic mutations, the effects of the novel splice junctions on transcript translation was evaluated by determining the transcript translation start site based on existing annotation in the Ensembl database (www.ensembl.org). A series of novel tumor-specific splice Frames were discovered using the methodology as described herein. For these tumor samples we found that splice Frame on-average contribute 2-90% neoantigenic amino acids to the expressed Framome of a tumor. FIG. 22 demonstrates the contribution of splice Frames to the Framome of multiple different tumors. The complete set of expressed Frames derived from short intra-exonic indels (frameshift indels), large structural genomic variants and splicing mutations for a single lung tumor is depicted in FIG. 23. This analysis demonstrates that splice Frames enlarge the size of a tumor's Framome, which provides improved opportunities for design of personalized neoantigen-based immunotherapies.

### Example 5: Use of Long-Read cDNA Sequencing to Enhance Detection of Splice Frames

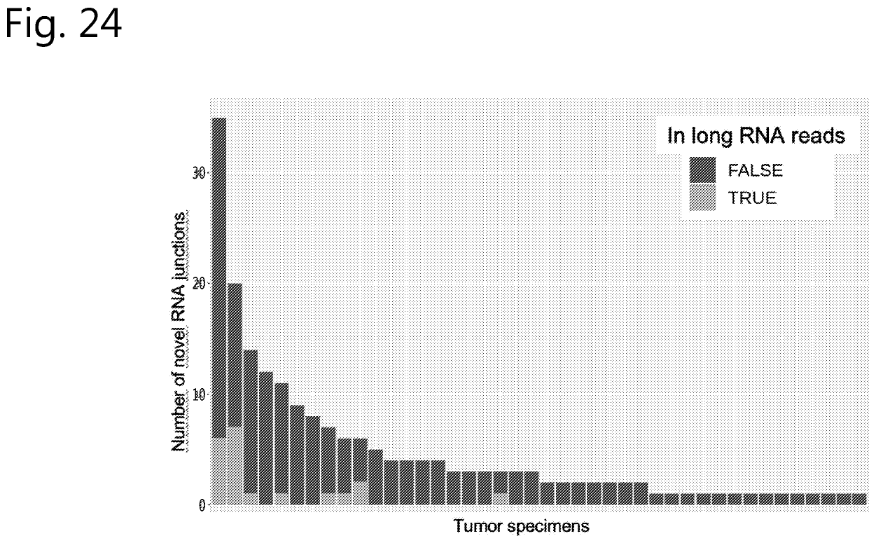

Novel methodology is provided to accurately determine the neoantigenic sequences resulting from genetic mutations that lead to splice aberrancies in a tumor sample. The use of long-read transcript sequencing to detect complete novel transcript sequences that lead to Frame neoantigens, as described herein, is a preferred method. The short-read RNA sequencing data derived from tumor specimens (amongst others lung, AML, pancreas) were evaluated for the presence of novel transcript splice junctions in the vicinity of possible gain-of-splice (GOS) mutations. In a subsequent step the presence of each novel short-read RNA junction was determined in corresponding long-read transcript sequencing data of the same sample. For only a fraction (<10%) of the novel short-read RNA GOS splice junctions, corresponding long-read transcript support could be obtained (FIG. 24). Many of the splice junctions that were uniquely found in short-read RNA data, were a result of mismapping of short-read RNA sequences in repetitive intronic genomic regions (FIG. 25). This indicates the value of an additional orthogonal validation step using long-read sequencing data to identify true-positive novel transcript splice junctions.

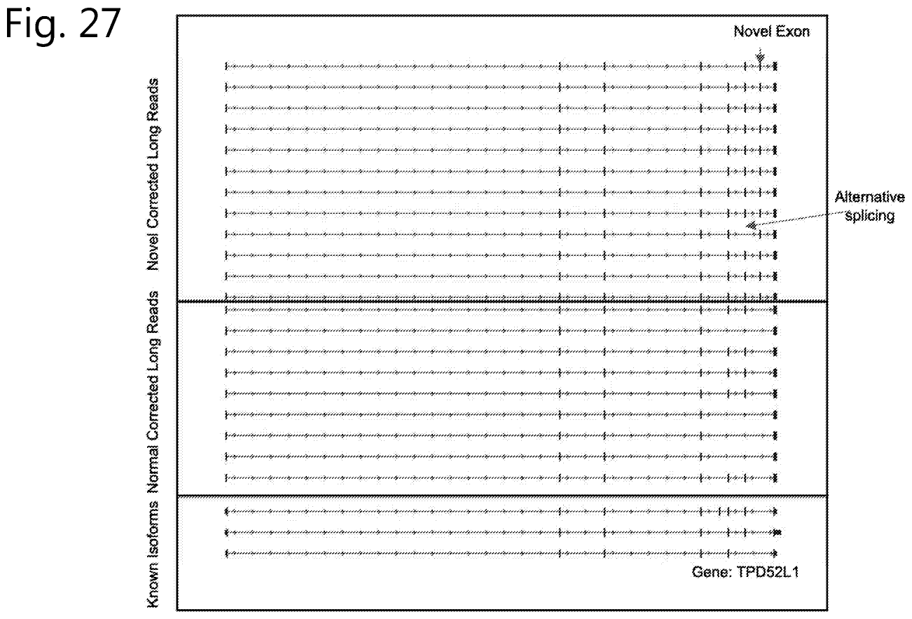

An important step with respect to the identification of splice Frames involves the prediction of the splice Frame peptide sequence. To determine the splice Frame peptide sequence, it is useful to know the translation start and the exact exonic structure of each transcript sequence (FIG. 26). Prior work has demonstrated that inferring transcript structure from short-read RNA sequencing data is a complicated process involving assembly of entire transcript sequences from short sequence reads (see e.g. Hölzer & Marz, GigaScience, Volume 8, Issue 5, May 2019). In particular, resolving alternative isoform structures from short-read RNA sequencing data is suboptimal. Instead, long-read RNA sequencing data enables immediate insight into the structure of each individual expressed transcript sequence, including rare isoforms (Workman et al, Nature Methods volume 16, pages 1297-1305 (2019)). An example of the complete exonic structure of mRNA transcripts involving novel splice junctions caused by a tumor-specific mutation is shown in FIG. 27. For each of the known and novel transcript sequences a prediction of the resulting splice Frame sequence can be accurately determined by (i) aligning the sequence of each individual transcript to the human reference genome (or a tumor-specific variant of the human reference), and (ii) determine the most 5′ translation start site for each individual transcript sequence based on translation start site annotation (e.g. from Ensembl), (iii) generating a complete transcript sequence by concatenating all exons into a single sequence string, (iv) translating the DNA sequence from the translation start site onwards until a stop codon is hit, (v) subtracting the N-terminal known part of the resulting protein sequence to determine the neoantigenic splice Frame sequence (FIG. 26).

### Example 6

The following steps describe an exemplary design of a Framome vaccine based on a cancer patient's mutation report.
1. Extract all somatic SVs, SNVs, and indels from the mutation report derived from cancer Whole Genome Sequencing.
2. Determine the expression of said mutations identified in the mutation report, by means of RNA sequencing.
3. Determine the entire transcript structures for messenger RNAs that contain frameshift mutations or other mutation types, e.g. using long-read RNA sequencing of poly-(A) selected mRNAs.
4. Project them onto the reference human genome sequence or onto tumor-specific reconstructed genome contigs to derive the resulting new open reading frame peptides Koster, J. & Plasterk, R. H. A. A library of Neo Open Reading Frame peptides (NOPs) as a sustainable resource of common neoantigens in up to 50% of cancer patients. Sci. Rep. 9, 6577 (2019).
5. Remove those that cause a new open reading frame shorter than N amino acids, where N can be set at 4, 5, 6 or more amino acids.
6. Screen the resulting newly encoded peptides against the products of the human ORFeome and filter out those that have a match of more than M amino acids, to avoid self-antigens, where M can be set at 5, 6, 7 or more amino acids. The remaining peptide sequences are referred to as Frames.
7. Rank the Frames by the criteria mentioned above.
8. When the vaccine consists of synthetic long peptides, the top ranking sequences defined under point 5 will be considered with the sum of the length of the top ranking sequences being <Q amino acids, where Q can be set at a practical number, e.g. 300 amino acids. Frames longer than 30 amino acids will be covered by a tiling array of 30-mer synthetic peptides, so that no epitope is lost because it happens to be on the edge of a single peptide.

### Example 7

### 1 Introduction

To identify the full repertoire of NOPs expressed by tumors, we developed FramePro, a genomics and bioinformatics software package that characterizes the framome—a set of all NOPs expressed by a tumor as a result of genetic mutations in cis. FramePro integrates whole genome sequencing (WGS) with long- and shortread RNA sequencing to detect full-length transcripts encoding NOPs at single-molecule resolution, thereby accounting for isoform diversity. FramePro was applied to 61 tumors across six cancer types, providing a comprehensive picture of expressed NOPs for each tumor sample. We describe an uncharacterized class of neoantigens, referred to as ‘hidden’ NOPs in which a known protein coding gene drives transcription and translation of a usually non-coding region of the genome which has been placed downstream via an SV. We demonstrate that transcripts encoding hidden NOPs are translated into proteins and that peptides derived from hidden NOPs can bind to MHC class I molecules and were found to generate memory T-cell responses in a lung cancer patient. Of note, hidden NOPs represent a major source of neoantigenic amino acids in most tumors. Taking the hidden NOPs together with those derived from frameshift indels, fusion genes, splice mutations and stoploss mutations, the framome size can reach up to ˜2000 amino acids for tumors across major cancer types. This large source of potentially highly immunogenic, long and tumor-specific peptide sequences represents an attractive target for personalized immunotherapy.

### 2 Results

### 2.1 Whole-Genome and Full-Length Transcriptome Sequencing of 61 Human Cancers

Recent studies have evaluated personalized neoantigen cancer vaccines in early-stage clinical trials with a primary focus on missense neoantigens identified from exome sequencing [21, 22, 23]. To systematically extend the repertoire of possible neoantigens expressed in tumors, we here focus on the identification of neoopen reading frame peptides (NOPs) derived from novel open reading frames (neo-ORFs), resulting from cis genomic mutations, including genomic rearrangements, indel frameshifts, splice mutations and stoploss mutations. [10, 11, 17, 18, 19, 15, 12, 13, 14, 20].

As a basis for our analysis, we collected a series of 61 tumor samples from patients with non-small lung cancer, pancreatic cancer (i.e. pancreatic ductal adenocarcinoma), head and neck cancer, colorectal cancer, glioblastoma, and triple-negative breast cancer. Tumor samples and corresponding normal tissue or blood samples were subjected to deep whole genome sequencing (tumor WGS−100X) to identify all classes of somatic genetic changes based on an existing and validated analysis pipeline (4. Methods) [8]. We identified on average 26,287 (208-418,406) single-nucleotide variants (SNVs); 1,847 (65-24,160) short indels and 261 (3-2,417) structural variants (SVs) per tumor sample (FIG. 31).

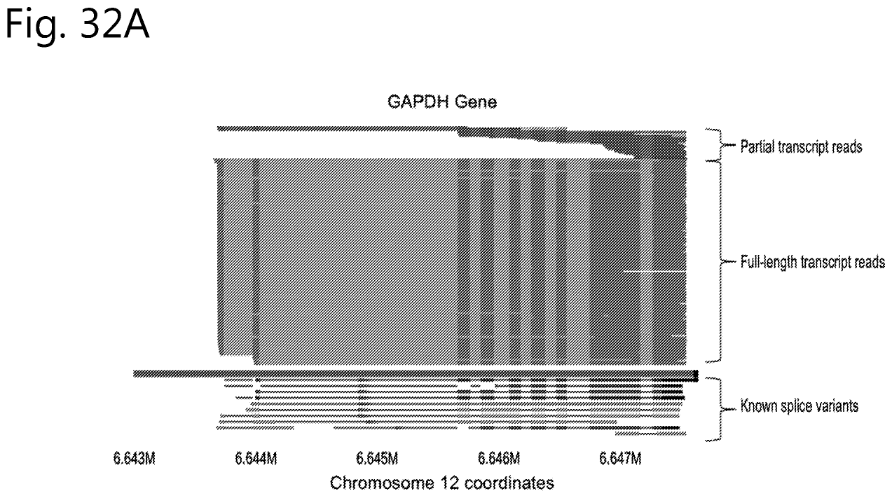

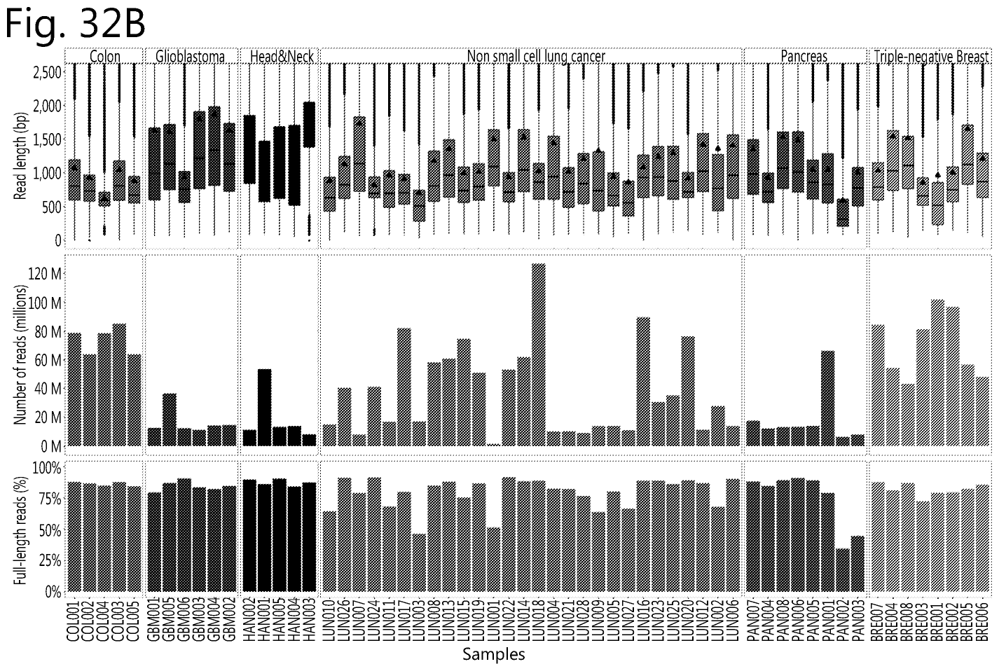

To characterize the effects of genomic changes on the tumor transcriptome, we performed RNA sequencing using a combination of conventional short-read RNA sequencing and long-read sequencing of mRNA transcripts (4. Methods). We developed a method to extract intact mRNAs from tumor samples, by a cDNA preparation process involving 3′-polyA and 5′-CAP selection. Double-stranded cDNA was sequenced on Nanopore sequencing devices reaching a throughput of about 1M-97M RNA sequences per sample. Up to 92.3% of long-read mRNA sequences spanned a full transcript molecule known in the Ensembl database, indicating the strength of the long-read data to determine complete transcript sequences at the single molecule level (FIG. 32). Short-read RNA sequences were used to correct the errors in long-read Nanopore sequences, generating high-quality sets of transcript sequences (4. Methods).

### 2.2 FramePro Identifies Expressed Neo-Open Reading Frames

Identification of possible neoantigens from sequencing data is often limited to the detection of coding mutations (e.g. by exome sequencing), followed by analysis of the expression of the identified genomic changes using short-read RNA sequencing. The neoantigenic peptide sequence is subsequently inferred from known transcript structures present in existing genome annotation data. However, a preferred method would be to directly determine peptide sequences based on the repertoire of expressed transcript isoforms in the tumor. We leveraged the WGS and short- and long-read RNA sequencing data as input for a novel bioinformatics pipeline (FramePro) to map complete tumor-specific transcript sequences caused by cis somatic mutations, including SVs, indels and SNVs within and outside coding regions.

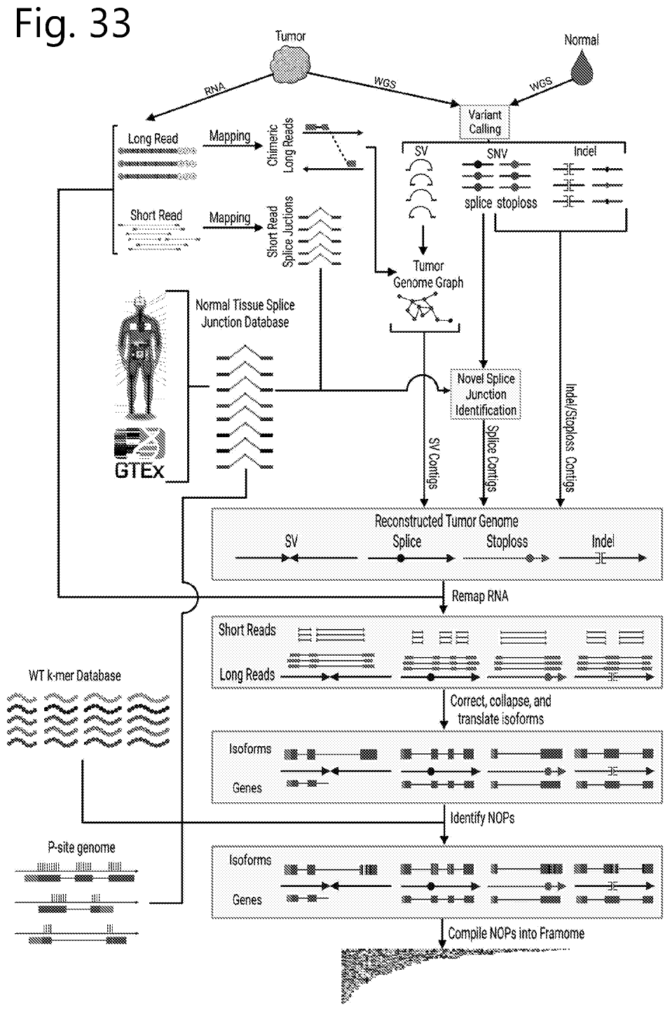

The FramePro analysis workflow comprises four steps that integrate somatic mutation data with transcriptome sequences to identify all neo-ORFs and corresponding NOPs (FIG. 33). In a first step, the collection of somatic small and structural variants is combined with chimeric long-read RNA mappings to construct tumor-specific contigs which together create a tumor-specific reference for each analyzed tumor sample. In a subsequent step, short-read and long-read RNA sequences are mapped to the tumor-specific reference to identify transcripts in the vicinity of, or overlapping a somatic mutation. In this step, the short-read RNA sequences are used to correct (splice-junction) errors inherent to long-read single-molecule Nanopore sequencing data. Subsequently, individual corrected transcript reads are used for in silico translation based on annotated translation start sites to derive entire protein sequences. In a final step, NOPs (or neo-epitopes from the NOPs) are derived from the protein sequences by trimming of the WT portions of each protein sequence. A detailed description of each step is provided in section 4.5.

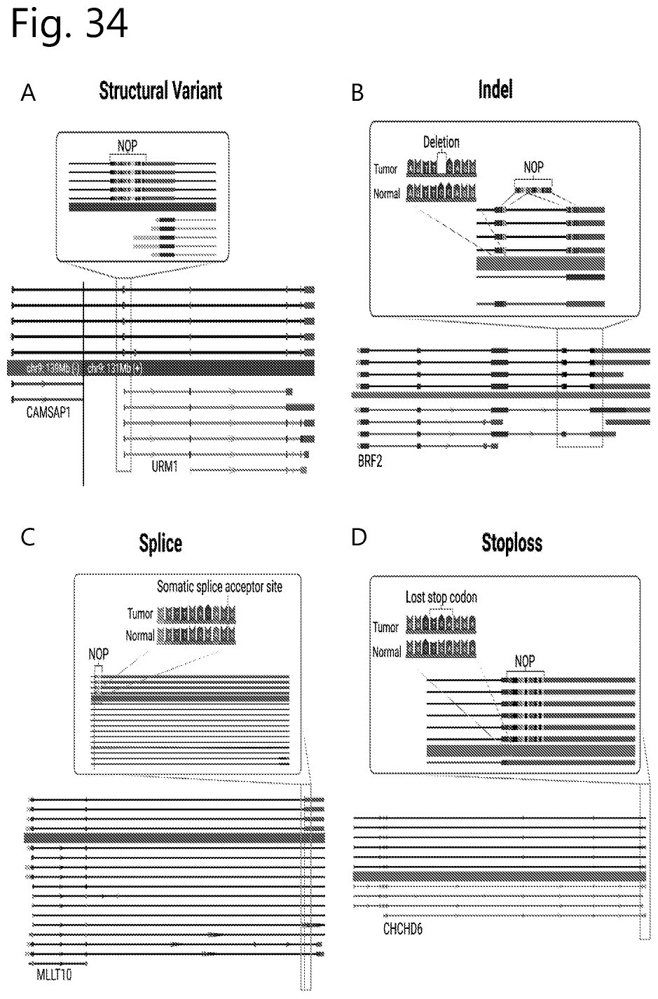

FramePro is the first tool to internally integrate full-length sample-specific transcript structures with variant protein effect prediction as well as the first tool to directly couple WGS with long-read transcriptome sequencing for the discovery and validation of SV-driven tumor specific isoforms. We used the FramePro analysis pipeline to analyze neo-ORFs and corresponding NOPs caused by SVs, frame-shift indels, splice mutations, and stoploss mutations across the entire tumor datasets of this study. An example of each class of NOP is provided in FIG. 34. One category of SV driven NOPs is a fusion gene event illustrated in FIG. 34A. While the canonical source of NOPs arising from fusion genes is through a mismatch in reading frame between the upstream and downstream genes, the example depicts translation initiation in the CAMSAP1 gene which is predicted to lead to a 27 amino acid NOP partially overlapping with the 5′-UTR of the URMI gene. An example of a NOP derived from a canonical exonic indel frame-shift is depicted in FIG. 34B, which displays a 49 amino acid NOP in the BRF2 gene in lung tumor sample LUN013 resulting from a single basepair deletion. Some of the frame-shift derived NOPs are found in tumor suppressor genes (23 NOPs across 18 samples), which form a source of shared NOP sequences [11]. We also identified NOPs caused by either mutations affecting known splice sites or mutations introducing new splice sites (FIG. 34C), as well as NOPs derived from mutations in known stop codons (FIG. 34D).

### 2.3 Hidden NOPs are a Novel Class of Neoantigens

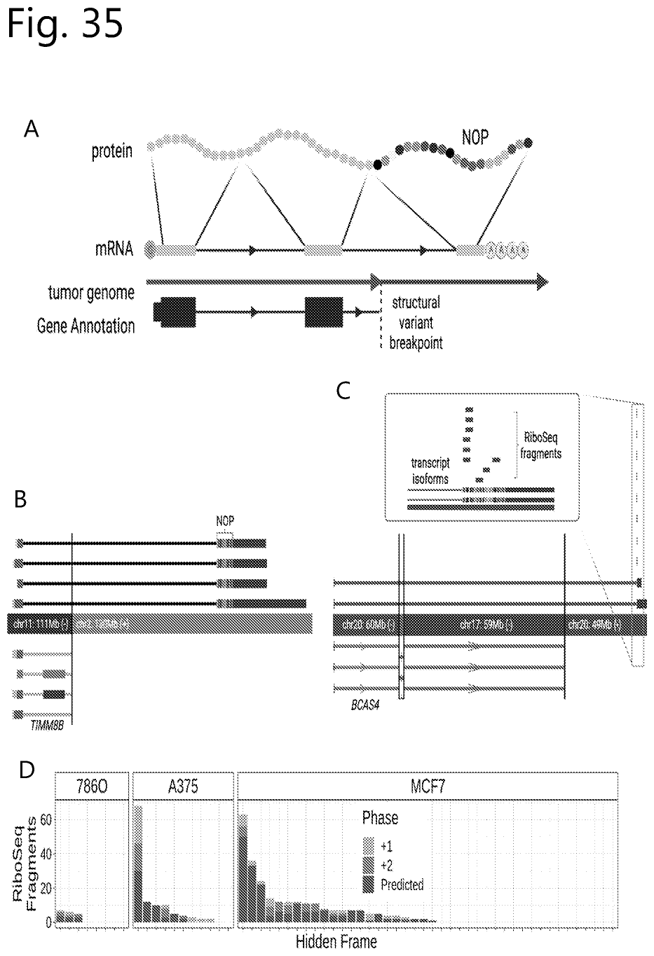

Gene fusions represent a frequent outcome of somatic SVs in cancer genomes and in-frame gene fusions can be drivers of tumorigenesis [24]. However, the majority of gene fusions represent a configuration where the 3′ partner gene is out-of-frame with the 5′ partner gene, creating a novel gene encoding a NOP [13] (FIG. 34A). We observed between 0-53 unique expressed NOPs corresponding to out-of-frame gene fusions per tumor sample, representing a substantial class of potentially neoantigenic sequences, particularly in tumors with high SV loads. An additional, yet largely uncharacterized configuration of genomic rearrangements involves the fusion between the 5′ part of a known gene and a non-coding genomic region. Based on in silico annotation of SV breakpoint junctions in the tumor samples, we observed that 2,864 (18%) of somatic SV breakpoint junctions involve a 5′-part of a known gene fused to a non-coding genomic region downstream of the SV breakpoint junction (FIG. 35A). Next, we analyzed the RNA sequences overlapping the SV breakpoint junctions in such regions and we observed that for 11% (320) of such SV junctions involving the 5′-end of a known gene and a non-coding region, breakpoint junction spanning transcripts were identified aligning with the 5′-end of a known gene and with a 3′-end that involves one or more novel cryptic exons. We have termed these chimeric transcripts and their resulting tumor-specific peptide products ‘hidden NOPs’. An example of a hidden NOP identified in lung tumor LUN004 is depicted in FIG. 35B, which shows the fusion of the 5′ exons of gene TIMM8B, located on chromosome chr11, coupled to novel cryptic exons encoded by a genomic region on chr2 which is not known to encode a gene. The novel chimeric transcript was confirmed by 25 long and corrected transcript reads.

To understand the translation of tumor-specific chimeric transcripts that encode hidden NOPs, we performed a FramePro analysis on human cell lines A375 (melanoma), MCF7 (breast adenocarcinoma), and 7860 (renal cell adenocarcinoma) resulting in the identification of 11, 22, and 8 hidden NOPs, respectively. For the majority of these hidden NOPs RiboSeq coverage was observed in the expressed non-coding region, with the majority of the RiboSeq reads indicating the expected reading frame that was inferred from the translation start site of the partner gene (FIG. 35D). An example of a highly-expressed hidden NOP is depicted in (FIG. 35C), showing RiboSeq reads in the expected reading Frame across the novel exons triggered by a set of genomic SVs in MCF7. In addition, we performed intracellular proteomics and immunopeptidomics analysis of A375 cells, demonstrating the presence of hidden NOP protein sequences and epitopes within and on the surface of A375 cells, respectively (FIG. 35D).

2.4 Many Tumors have Large Framomes

We identified 946 unique NOPs amongst the 61 tumor samples described in this work, and we classified the NOPs according to their genomic origin (FIG. 34, FIG. 35A). On average we identified 16 NOPs with a combined length of 369 amino acids per tumor sample (FIG. 36A). Across all analyzed cancers, we found that hidden frames were the major source of NOPs with 54% of novel amino acids arising from this source. Fusion genes and indels contributed 34% and 10% respectively, with the remaining 1% made up of the stoploss and splice NOPs. Taken together, hidden frames and fusion genes made up 88% of all NOPs identified. Different cancer types express NOP classes at different frequencies. We observed that glioblastomas often express hidden NOPs and gene-fusion NOPs (99% of novel amino acids), as a result of the high load of somatic SVs and a low number of exonic indels. For lung cancer, we found a higher amount of indel NOPs (19% of novel amino acids) than average reflecting the relative amount of frame-shift indels and SVs in this cancer type.

We have termed the entire collection of NOPs expressed by a specific tumor sample ‘the framome’. Representative examples of tumor framomes are given in FIG. 36B,C. Glioblastoma sample GBM005 expresses 80 unique NOPs, for a total of 1,785 amino acids, almost all of which are derived from somatic SVs. In contrast, the Framome of non-small cell lung cancer LUN013 represents 1,106 amino acids across 46 NOPs, many of which are a result of canonical frame-shift indels.

Expression level and clonality are features that can be used for selection of neoantigens as immunotherapy targets [25, 26]. The expression levels of mRNAs encoding NOPs were measured based on the long-read RNA sequencing data generated for each tumor sample and quantified as transcripts per million (TPM) FIG. 36D. Expression levels of NOPs largely fall into the distribution of the expression levels observed for other genes (FIG. 37A). A slight shift in the average expression of NOPs compared to missense variants was observed for some tumor samples which is likely an effect of nonsense-mediated decay (FIG. 37B) [27]. The variant allele frequency of the underlying somatic genetic changes was determined from the whole genome sequencing data and corrected for tumor purity FIG. 36D [8]. We observed that variants encoding NOPs have an average purity corrected VAF of 0.37, similar as other somatic variants in the analyzed tumor genomes (FIG. 37C).

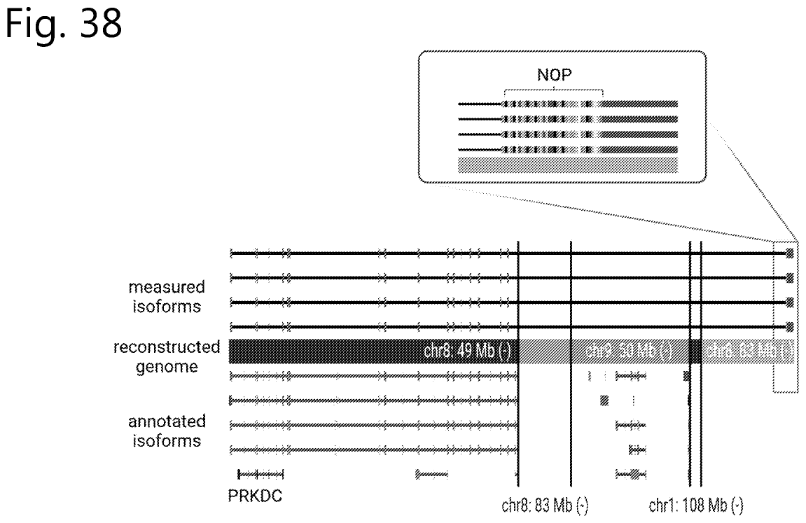

Complex chromosomal rearrangements, such as chromothripsis, are a frequent phenomenon in cancer genomes [28]. For 32% of the genomic events leading to hidden NOPs or out-of-frame gene fusions, the genomic connection between the 5′-end of the known gene and the non-coding genomic segment or downstream out-of-frame gene was formed by more than one genomic breakpoint-junction (FIG. 36E, FIG. 38).

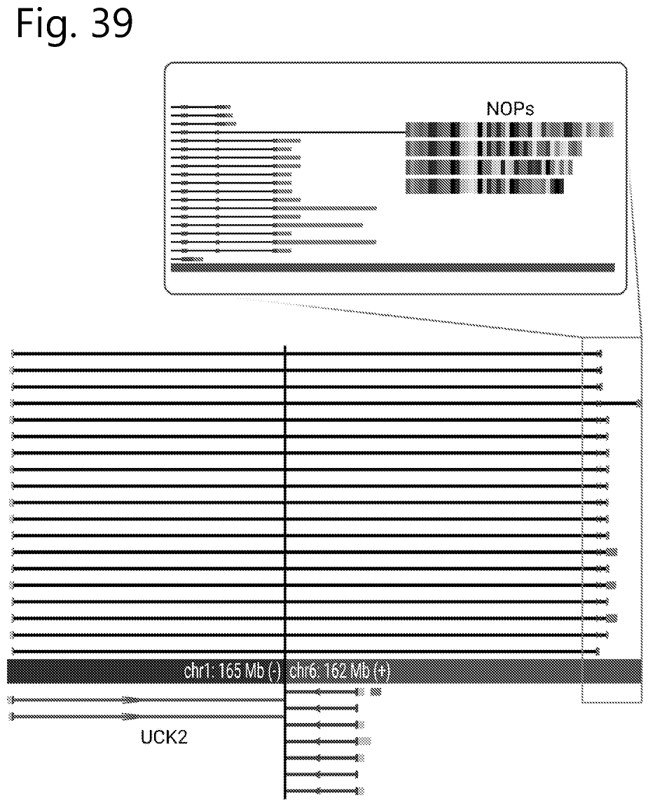

The analysis of single full-length transcript molecules using FramePro enabled us to identify the entire spectrum of transcript isoforms encoding a hidden NOP. The majority (67%) of SVs leading to hidden NOPs involve transcripts that encode a single unique NOP. However, we observed multiple instances of hidden NOPs that were caused by different transcript isoforms derived from the same genomic SV. For example, a hidden NOP in a triple negative breast tumor involved multiple splice isoforms encoding 4 different unique NOP sequences (FIG. 39). Isoform diversity may thus enlarge the neoantigenic potential of hidden NOPs.

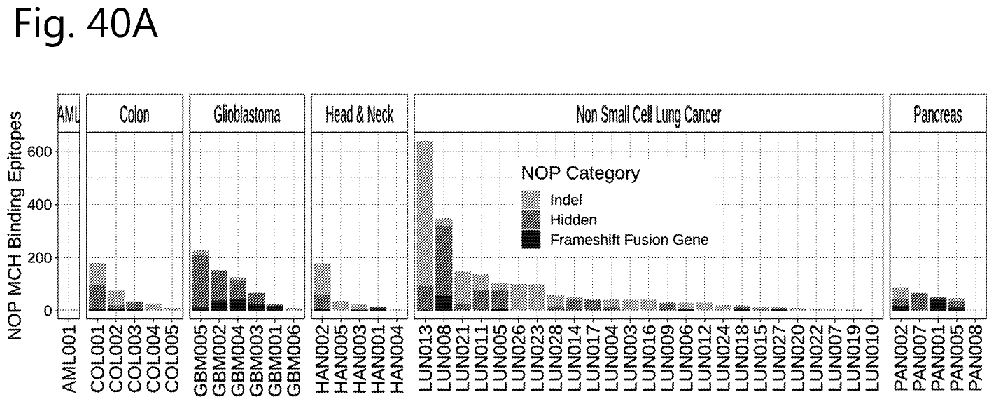

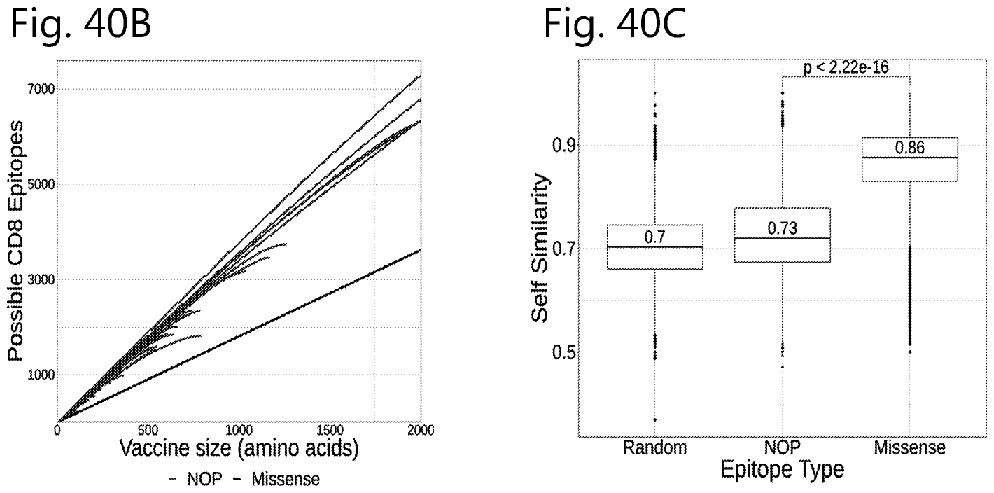

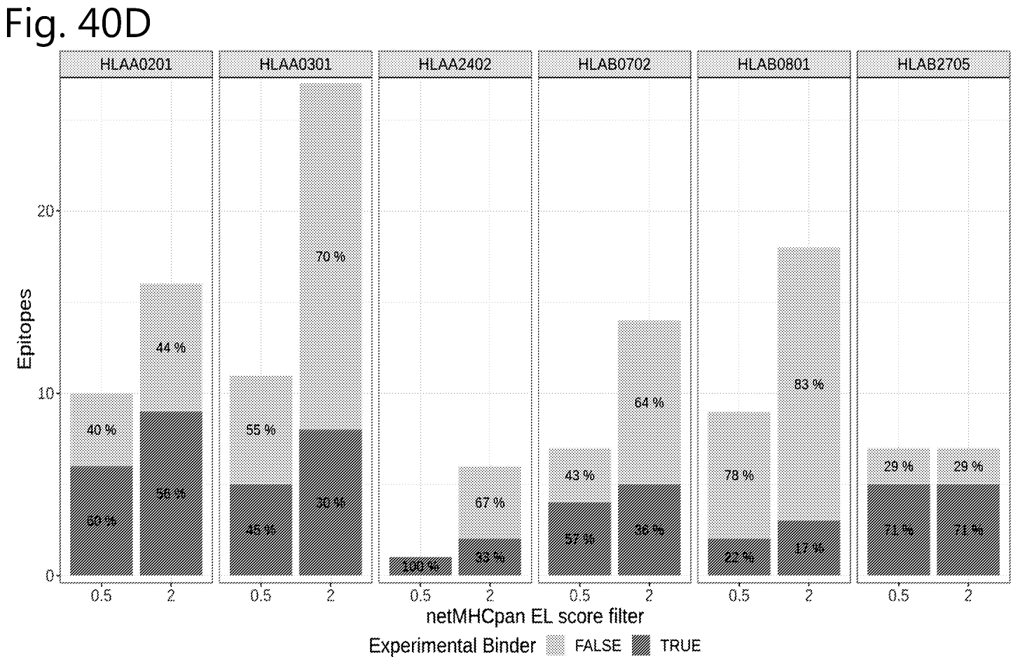

To understand the amount of possible HLA binding epitopes among NOPs expressed in tumors, we performed in silico characterization of HLA class I binding. To do so, we determined the HLA class I types for each individual tumor based on whole genome sequencing data, and the HLA types were used to predict binding epitopes within NOP sequences (Methods 4.6). The number of predicted binders is shown in FIG. 40A, which illustrates an average of 220 predicted binders per 1,000 amino acids of framome.

To understand whether a cancer vaccine based on NOPs would be advantageous with respect to the number of possible MHC class I epitopes, as compared to vaccines based on commonly used missense variants, we generated cancer vaccine designs as described in Methods 4.8. In FIG. 40B a comparison is made between the number of potential MHC class I epitopes for the two classes of antigens in the context of a neoantigen-based personalized therapeutic cancer vaccine designed in silico for each of the tumors reported in this study. This analysis shows that for many tumors a ˜2 fold increase in targeted epitopes can be achieved through the use of NOPs compared to missense variants. Targeting NOPs may allow not only for a superior quantity of targeted epitopes but also a superior quality of each epitope as there is increasing evidence that neoantigen dissimilarity to self proteins is important for effective immune response [29]. The long out-of-frame peptide sequences represented by NOPs are, in principle, fully tumor-specific and the same sequences should not be expressed in normal (non-tumor) cells. We determined the similarity to self for all 9-mers derived from NOPs and missense variants expressed by each of the 61 tumors analyzed in our study (Methods 4.7, FIG. 40C). This demonstrates that NOP epitopes are nearly as dissimilar from self as completely random epitopes (mean 0.7 vs 0.74), while missense epitopes which differ from wild-type epitopes by only a single point mutation are highly self-similar (mean 0.86). These results suggest that the length and foreignness of NOPs provide a potential advantage over missense mutations as immunotherapy targets.

2.5 Framome-Derived Epitopes Bind to Various HLA Alleles In Vitro and are Recognized by Memory CD8+ T Cells of Patients with Advanced NSCLC

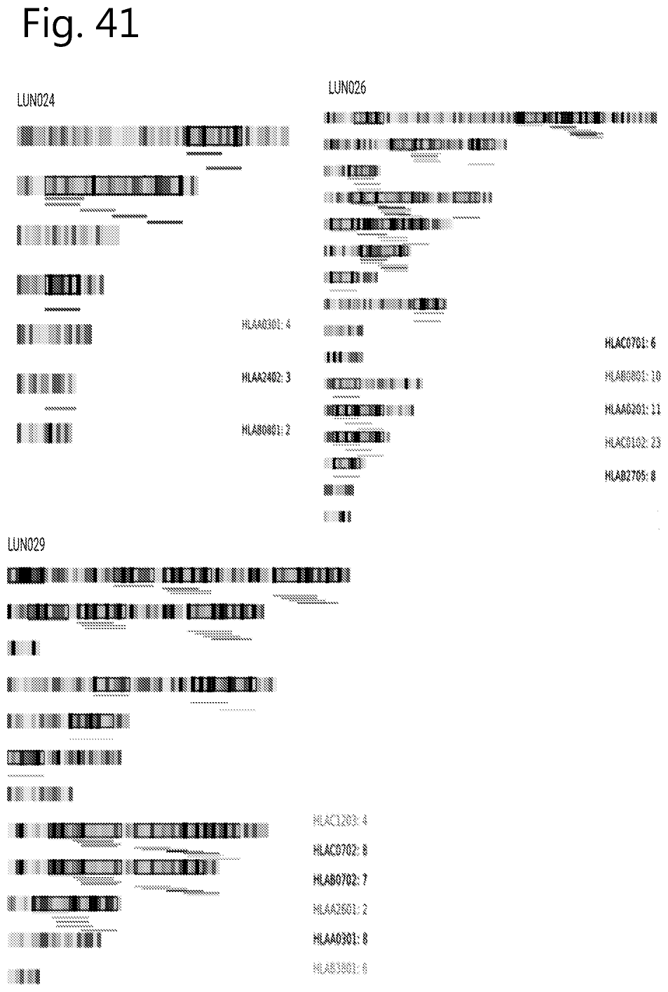

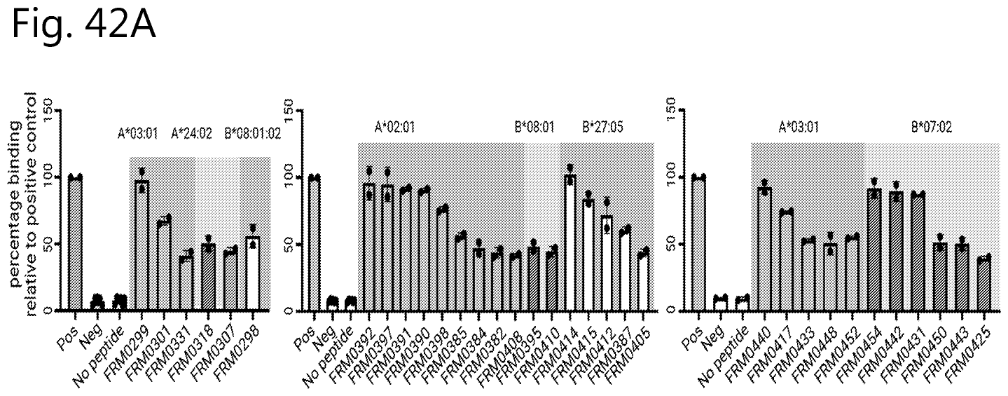

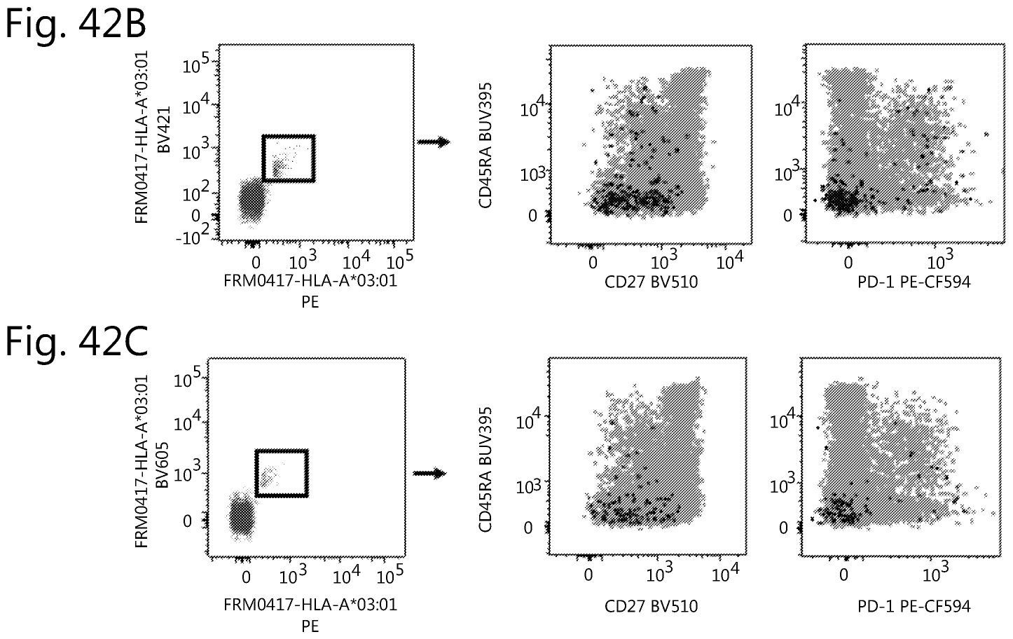

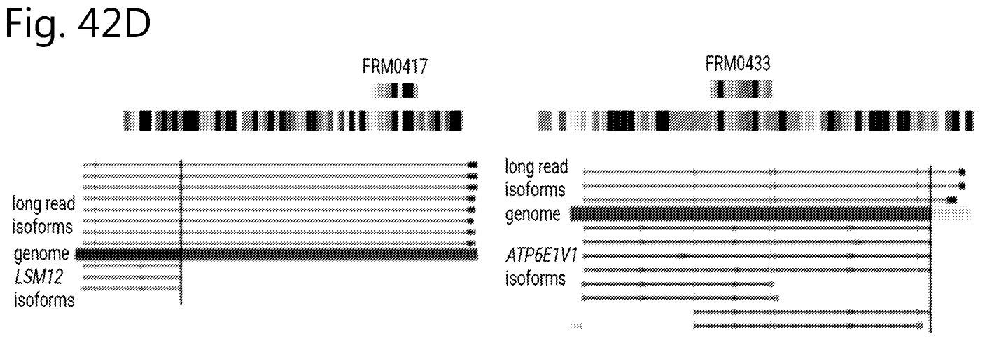

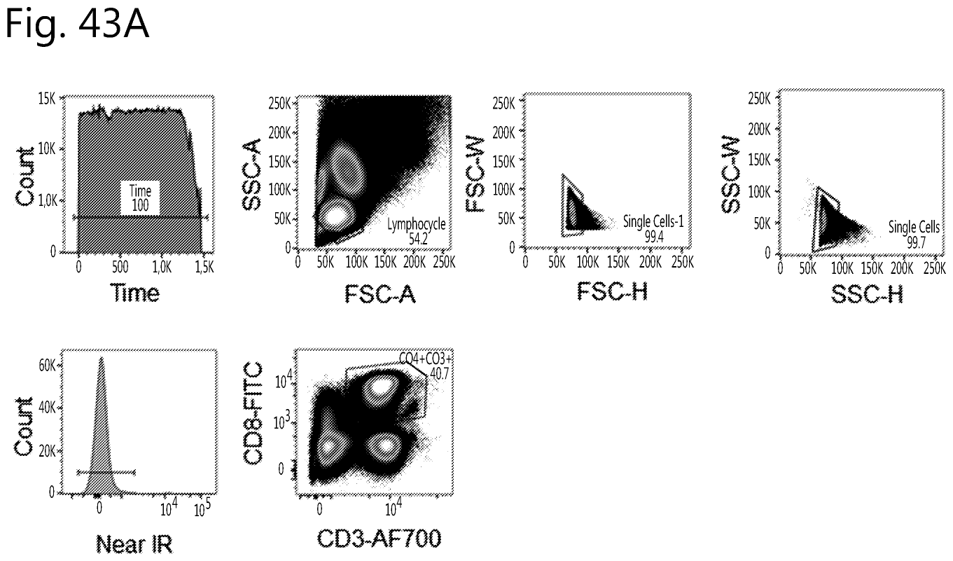

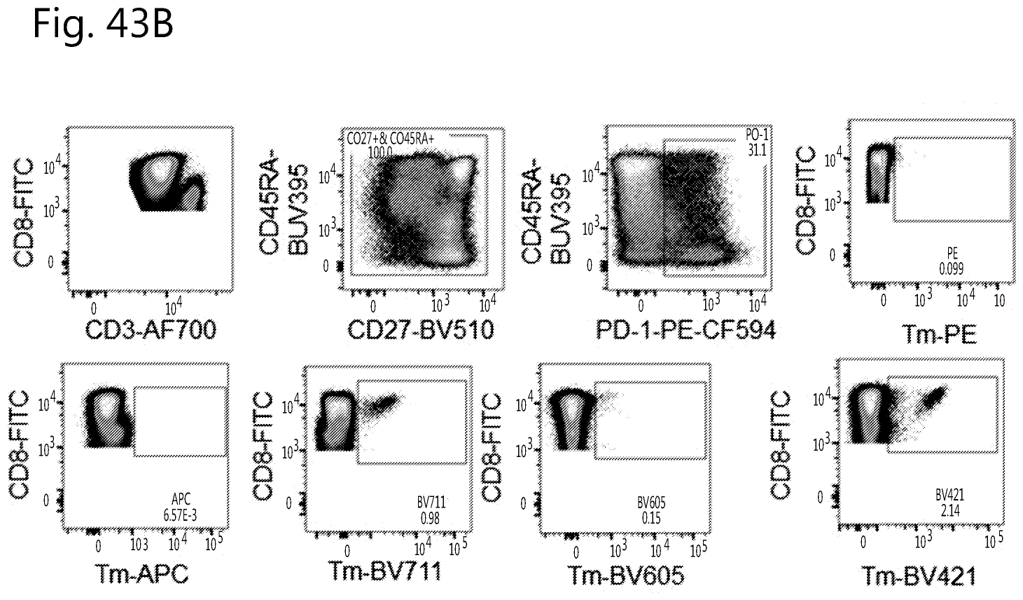

To further characterize the immunogenic properties of NOPs, we assessed the affinity of framome-derived epitopes to various HLA-A and -B alleles by performing in vitro HLA binding assays (Methods 4.11). First, we selected more than 30 epitopes derived from the framomes of each of three patients with advanced lung cancer (LUN024, LUN026, and LUN029) and we tested the binding of these epitopes using in vitro binding assays FIG. 41. As the framomes of patients LUN026 and LUN029 provided many predicted epitopes, we limited our selection to those with the highest predicted affinity (EL score below 2) for each relevant HLA allele. In vitro binding analysis revealed a number of epitopes binding to HLA-A and HLA-B specific for each patient FIG. 42A. The correlation between predicted and the actual in vitro binding to various HLA alleles FIG. 40D, showed that the prediction had higher confidence if the EL score was below 0.5. The best correlation between predicted and the actual binding affinity was observed for HLA-A*24:02 allele with all predicted peptides with EL score below 0.5 binding to this allele also in vitro. The correlation between predicted and the actual binding in vitro ranged between 22% (HLA-B*08:01) and 71% (HLA-A*27:05) FIG. 40D. In the next step, we generated fluorescently labeled HLA tetramers carrying epitopes with at least 40% binding affinity, as determined by the in vitro binding assays. These tetramers, specific to each patient's HLA allele, along with relevant positive and negative controls, were then used to stain low frequency antigen specific CD8+ T cells within the PBMC population of each patient's peripheral blood using combinatorial coding (Methods 4.11). Additionally, antigen specific CD8+ T cells recognizing individual epitopes were phenotyped to determine their antigen experience status (for the gating strategy see FIG. 43). We detected the presence of effector memory type (CD8+CD45RA−, CD27−/dim) T cells in the blood of patient LUN029, specific for two epitopes, FRM0417 FIG. 42B and FRM0433 FIG. 42C. Each of the epitopes originated from a different NOP FIG. 42D, categorized as a hidden NOP. There were no antigen specific CD8+ T cells found in the repertoire of patients LUN024 and LUN026 (data not shown). These data confirm that antigens derived from hidden NOPs can bind to various HLA alleles and can induce antigen specific immune responses in some patients with cancer.

### 3 Discussion

The importance of neoantigens for cancer immunotherapy has become clear from multiple studies that have highlighted the relation between tumor mutational burden and the effectiveness of T-cell checkpoint immunotherapy [30]. Initial work has particularly emphasized the role of exonic point mutations leading to single amino acid changes (i.e., missense variants) in checkpoint immunotherapy response [31]. Further studies have demonstrated that novel neoantigenic peptides derived from frame-shift indels, which are highly different from self, contribute to the immunogenic phenotype of cancers and positively correlate to checkpoint inhibitor response [9]. In addition, novel tumor-specific peptide sequences derived from splice aberrancies and gene-fusions have been shown to provide additional sources of possible neoantigenic NOP sequences across cancer types [14, 32, 33]. Complementary experimental studies have confirmed the strong immunogenic properties of NOPs derived from frame-shifts, including their capacity to trigger CD4+ and CD8+ T-cell responses and tumor growth delay in model systems [10, 34]. The long and foreign peptides represented by NOPs may be preferred targets for immunotherapies, stressing the need for a robust method to identify all classes of NOPs from a small tumor biopsy.

The work described here provides a technological and bioinformatics framework to exploit the full potential of neo-open reading frames encoded in the tumor genome as a result of cis-acting somatic mutations. Identification of the full spectrum of expressed NOPs in tumors requires whole genome sequencing as basis complemented with RNA sequencing to map mutated transcripts. Only whole genome sequencing captures the complete catalogue of somatic mutations arising in cancer genomes, including SNVs, indels and SVs [8]. Although commonly used exome sequencing is an efficient technology for detection of exonic mutations (e.g., frameshift indels) in tumor samples, it falls short with respect to identification of intronic and intragenic variants and SVs. For example, splice-site creating mutations are a known source of neoantigenic sequences, yet such mutations often reside outside of known exons captured by exome sequencing [18].

Our work demonstrates that SVs provide a rich source of possible cancer neoantigens, beyond well-described neoantigenic sequences derived from fusion genes [14]. We performed a systematic analysis of the effects of SVs on the cancer transcriptome and find that SVs often drive expression of non-coding genomic regions via fusion with the 3′-end of a known gene. We designate these as hidden NOPs as their existence cannot be identified from genome sequencing alone, but requires the integrated analysis of cancer transcripts sequences with somatic SVs in the cancer genome. By comparing the contribution of NOPs derived from splice mutations, stop loss mutations, frameshift indels, and SVs, we observed that >50% of the amino acid sequences contributed by NOPs are derived from hidden NOPs caused by SVs. Additionally, we validated the relevance of SVs as neoantigens as we identified hidden NOP specific memory type CD8 T cells in the blood of a patient with advanced NSCLC. Personalized neoantigen-based immunotherapy strategies targeting tumors with a high level of SVs (e.g., glioblastoma, TN breast), or with both high SV and high indel count (e.g. lung cancer) would benefit from a neoantigen discovery approach as outlined here. Personalized cancer vaccines are currently studied in many clinical trials worldwide [3], and the basis for such vaccines is formed by sequencing of the tumor exome. We propose that a complete analysis of the cancer genome will enable optimal design of personalized cancer vaccines, thereby leveraging the full neoantigenic potential of a tumor.

In addition to genomic analysis of the tumor, faithful mapping of mutation-derived transcripts encoding possible neoantigens allows one to precisely determine tumor-specific peptide sequences. The conventional approach for determining the expression of somatic variants in tumor samples is based on short-read RNA sequencing, where allele-specific expression can be measured from the RNA sequences covering a specific genetic mutation. Although such measurement provides immediate insight into the expression level of a specific genetic mutation, it does not provide a complete view on the sequence context of the expressed mutations. The wide diversity of transcript isoforms encoded by the human genome has become apparent through full-length transcript sequencing [36]. Direct mapping of the isoforms of a gene would be a preferred approach to infer neoantigenic peptide sequences, rather than the commonly used approach to use existing transcript annotations. Here, we demonstrate the value of long-read transcriptome sequencing and integrating the long-read transcript sequences with somatic mutations identified through whole genome sequencing. The combined approach of whole genome and long-read transcriptome sequencing enables analysis of neoantigenic sequences derived from individual transcript sequences based on the identification of translation start sites and accurate transcript structure and sequence. Our current approach involves the use of short-read RNA sequencing to refine transcript splice-junction sequencing, but we expect that future generations of long-read sequencing will make such an approach obsolete.

In conclusion, we here present a universally applicable FramePro methodology that enables systematic identification of neo-open reading frames and corresponding NOPs resulting from somatic mutations in a tumor genome. We propose that upcoming personalized cancer immunotherapies include a comprehensive analysis of possible neoantigenic sequences expressed by the tumor, as a basis for therapy design. The outcome of clinical trials based on such neoantigen detection approach will provide experimental evidence for the relative contribution of different neoantigen classes to the tumor immunophenotype, as well as their relevance for therapy effectiveness.

### 4 Methods

### 4.1 Patient Samples

Fresh frozen tumor biopsies and corresponding blood samples or normal control tissue were obtained from different clinical centers. Informed consent and ethical approval was obtained for each sample for studying tumor DNA and RNA sequencing information. Patient samples were obtained under studies OLSO41-202100773 Framoma (Oncolifes, University Medical Center Groningen), AMC 2014 181 BioPAN (Amsterdam UMC), IRBdm21-018 (Netherlands Cancer Institute), 09H050190 (LREC, University of Liverpool), Pro000074343 (Duke University), XXX (Erasmus Medical Center Rotterdam), NCT01792934 (Radboud University Medical Center).

### 4.2 Whole Genome Sequencing

Genomic DNA was isolated from tumor biopsies and control tissue (blood or adjacent normal tissue) using Qiagen DNeasy. As input, 50-200 ng of DNA was sheared to an average length of 450 bp by Covaris and standard TruSeq Nano LT library preparation (Illumina) with 8 PCR cycles was performed. Barcoded libraries were sequenced on Illumina NovaSeq instruments with 2×151 bp settings, to an average coverage depth of 100× (tumor samples) and 35× (control samples). FASTQ generation was done using Illumina bcl2fastq (v2.20.0.42). Sequencing reads were mapped to human reference genome GRCh37 using BWA (version) with settings XXX. Somatic genomic variants were called from aligned sequencing data using a custom pipeline [8] (https://github.com/hartwigmedical/pipeline5/tree/master/cluster/src/main/java/com/hartwig/pipeline).

### 4.3 Short Read RNA Sequencing

Total RNA was isolated from fresh frozen tumor samples using NucleoSpin RNA isolation (Machery Nagel). cDNA library prep was performed according to a standard protocol using 100 ng of total RNA, which was chemically sheared for 7 minutes. Resulting cDNA was PCR amplified for 15 cycles. Libraries were sequenced on an Illumina NovaSeq system to a minimal depth of 50M paired reads (100M tags) per cDNA library based on 2×151 bp settings. FASTQ generation was done using Illumina bcl2fastq (v2.20.0.42). cDNA sequencing reads were mapped to the human reference genome GRCh37 using STAR (version) with settings XXX. Further processing of short cDNA sequencing data was done as described in section 4.5.

### 4.4 Long Read RNA Sequencing

About 500 ng to 2 microgram of total RNA was used as input for double stranded cDNA preparation using TeloPrime Full-Length cDNA Amplification kit V2 (Lexogen) according to manufacturer's specifications. TeloPrime selects mRNA molecules containing a 5′ CAP and a 3′-poly-A tail. For some samples poly-A selected RNA was used as input for TeloPrime cDNA preparation. For those cases, selection of poly-A mRNA was performed using Dynabeads mRNA Purification kit (Invitrogen) and between 20-100 ng of poly-A selected mRNA was used as input for TeloPrime. Between 11-20 PCR cycles were performed for each sample. Double stranded cDNA was used as input for preparation of a Nanopore sequencing library using SQK-LSK109. Libraries were sequenced on GridION or PromethION systems (Oxford Nanopore Technologies) to a depth of between 20M-100M reads. Long cDNA Nanopore reads were mapped to human reference genome GRCh37 using Minimap2 (version). Further processing of long-read Nanopore cDNA sequencing data was done as described in section ‘Frame Pro methodology’.

### 4.5 FramePro Pipeline

All core steps in the FramePro pipeline including genome reconstruction, RNA isoform identification, isoform translation prediction, and NOP identification were implemented in python and packaged into the framepro package. Nextflow [37] was used to integrate these steps with RNA mapping and read extraction into the framepro-nf pipeline.

### 4.5.1 Tumor Genome Reconstruction

To identify neo-ORFs and corresponding NOPs, a tumor-specific reference genome was generated for each sample onto which long and short read RNA could be aligned. These tumor-specific reference genomes consisted of collections of contigs which captured the local effects of somatic mutations. For SVs, these contigs were identified through a combination of an RNA-naive approach and an RNA-guided approach.

To construct RNA-naive tumor SV contigs, SVs for a given sample were collected in breakend format. All protein coding genes hit by an SV in were identified. For each of these genes, a contig was constructed by starting ϵₛₜₐᵣₜ basepairs (default 1 kB) upstream of the first start codon and including the gene sequence up to the first SV breakend within the gene. The sequence downstream of this breakend was appended to this contig by crossing the SV to the mate breakend and continuing in the orientation specified until another SV breakend was encountered and crossed. SVs were removed from the list of SVs once crossed. This process was carried out until ϵ_{maxL} basepairs (default 2 Mb) were appended downstream of the original gene segment. Each contig assembled in such a manner represents a possible local region of the tumor genome which is consistent with the SVs identified through tumor/normal WGS. By starting at the 5′ end of protein coding genes and extending downstream a distance longer than the typical range of transcription, all gene fusions and hidden frames whose protein expression may be driven by the starting gene can be identified once full-length transcripts are aligned to these contigs.

This RNA-naive approach can correctly resolve regions downstream of protein coding genes which involve simple SVs because it follows a linear path through next-nearest breakends. For more complex regions, such as occurs in chromothripsis, breakage fusion bridges, etc., an approach which utilizes information at the RNA level is used. Instead of starting with genomic events (SVs) which are not yet known to affect RNA transcripts, this approach takes sets of ungapped chimeric RNA alignments and attempts to explain their apparent transcript structure at the genome level through SVs. The set of contigs which explain these transcript structure changes are then appended to the reconstructed tumor reference genome after collapsing contigs redundant with the RNA-naive approach.

The RNA-guided approach starts with the alignment of RNA to a base reference genome as specific in section 4.4 and proceeds as illustrated in FIG. 44. Let R be the set of RNA reads with at least one alignment within ₛᵥ base pairs (default 200 kB) of an SV breakend and which have at least one supplementary or secondary alignment. Let Aᵣ be the set of primary, supplementary, and secondary alignments of read r∈R. For given alignment aᵢ ∈Aᵣ let a_{iqs} be the start position within the query sequence of aᵢ as measured from the 5⁰ end of the RNA read r. Similarly, let a_{iqe} be the query end position. Let aᵢₛ, aᵢᵣₛ, and aᵢᵣₑ be the reference alignment strand, strand-specific reference start, and strand-specific reference end of aᵢ, respectively where aᵢᵣₛ≤aᵢᵣₑ if and only if aᵢₛ is positive. A set Qᵣ of ordered sets p of alignments in Aᵣ can be defined as:

$${Q}_{r} = \{ q \vert {q}_{i} ∈ Ar , {q}_{iqs} < {q}_{i} + {1}_{qs} and \vert {q}_{i + {1}_{qs}} - {q}_{{i}_{qs}} \vert < {ϵ}_{p} ∀ i ∈ ( 1 , … , ‖ q ‖ - 1 ) \} \qquad ( 1 )$$

The elements of Qᵣ represent collections of consecutive segments of the read r which are non-linearly aligned to the reference genome. A gap or overlap buffer of ₚ is utilized to allow for soft or hard-clipping, erroneous indels, and homology at the beginning and ends of the alignments. To arrive at a non-redundant (excluding prefix/suffix paths) set of chimeric RNA paths for read r, the set Pᵣ can be defined as:

$${P}_{r} = \{ p \vert p ∈ {Q}_{r} , p 6 ⊂ q ∀ q ∈ {Q}_{r} ∖ \{ p \} \} \qquad ( 2 )$$

To find possible underlying tumor contig regions from which the proposed chimeric RNA structures within Pᵣ may have arisen, it is necessary to find paths of SVs which connect the beginning and ends of consecutive chimeric alignments, referred to as chimeric introns. Each chimeric RNA path p in Pᵣ contains a set Mₚ of size kpk−1 such chimeric introns m where m_{L} and m_{H} represents the lower and upper alignments on each side of the chimeric intron. This set Mₚ can be defined as:

$${M}_{p} = ( m \vert {m}_{L} = {p}_{i} and {m}_{H} = {p}_{i + 1} ∀ i ∈ ( 1 , … , kpk - 1 ) ) \qquad ( 3 )$$

A chimeric RNA path is considered supported by somatic genomic events if there is a conceivable path through the tumor genome which connects the end of the first chimeric intron alignment to the start of the second chimeric intron alignment for each chimeric intron in the path. To determine this for each path, a directed graph Gₚ is constructed which represents all possible connections within the tumor genome. The end/start loci of each chimeric intron can then be anchored onto Gₚ in order to find a valid path across the chimeric intron. To construct this graph, let the sample SVs be represented by a set B of breakends b where b_{c}, bₚ, bₛ, bₘ are the breakend chromosome, position, strand, and mate breakend, respectively. Let the vertex set V (Gₚ) consist of vertices v where v_{c}, vₚ, vₛ correspond to chromosome, position, and strand of genomic loci. Let two identical sets of breakend vertices be V_{source} and Vₛᵢₙₖ be defined as:

$${V}_{sources} = \{ v \vert {v}_{c} = {b}_{c} and {v}_{p} = {b}_{p} and {v}_{s} = {b}_{s} ∀ b ∈ B \} \qquad ( 4 )$$

$${V}_{sinks} = \{ v \vert {v}_{c} = {b}_{c} and {v}_{p} = {b}_{p} and {v}_{s} = {b}_{s} ∀ b ∈ B \} \qquad ( 5 )$$

Let the sets of lower-alignment and upper-alignment chimeric intron vertice sets be defined as:

$${V}_{L} = \{ v \vert {v}_{c} = m {L}_{c} and {v}_{p} = m {L}_{p} and {v}_{s} = m {L}_{s} ∀ p ∈ {P}_{r} ∀ m ∈ {M}_{p} \} \qquad ( 6 )$$

$${V}_{H} = \{ v \vert {v}_{c} = m {H}_{c} and {v}_{p} = m {H}_{p} and {v}_{s} = m {H}_{s} ∀ p ∈ {P}_{r} ∀ m ∈ {M}_{p} \} \qquad ( 7 )$$

The vertex set V (Gₚ) is then:

$$V ( {G}_{p} ) = {V}_{sources} ⋃ {V}_{sinks} ⋃ {V}_{L} ⋃ {V}_{H} \qquad ( 8 )$$

Two types of connections between genomic loci are possible within the rearranged tumor genome: those which occur between points on the same strand of the same chromosome in the normal reference genome and those which occur due to SVs. The edge set E_{WT} represents WT connections which point from source vertices to sink vertices:

$${E}_{WT +} = \{ ( v , u ) \vert v ∈ {V}_{sources} , u ∈ {V}_{sinks} , {v}_{c} = {u}_{c} , {v}_{s} = + , {v}_{s} = - , {v}_{p} \leq {u}_{p} \} \qquad ( 9 )$$

$${E}_{WT -} = \{ ( v , u ) \vert v ∈ {V}_{sources} , u ∈ {V}_{sinks} , {v}_{c} = {u}_{c} , {v}_{s} = - , {v}_{s} = + , {v}_{p} \geq {u}_{p} \} \qquad ( 10 )$$

$${E}_{WT} = {E}_{WT +} ⋃ {E}_{WT -} \qquad ( 11 )$$

The edge set E_{SV} represents connections between breakpoints due to SVs which point from sink vertices to their partner breakend source vertices:

$$ESV = \{ ( v , u ) \vert v ∈ {V}_{sinks} , u ∈ {V}_{sources} , {v}_{c} = {b}_{c} , {v}_{p} = {b}_{p} , {v}_{s} = {b}_{s} , {u}_{c} = {b}_{m c} , {u}_{p} = {b}_{m p} , {u}_{s} = {b}_{m s} ∀ b ∈ B \} \qquad ( 12 )$$

The edge set E_{M} represents the connections between lower chimeric intron alignments to sink breakend loci as well as the connections between source breakend loci to upper chimeric intron alignments (equations 13-17):

$${E}_{L +} = \{ ( v , u ) \vert v ∈ {V}_{L , u} ∈ {V}_{sinks} , {v}_{c} = {u}_{c} , {v}_{s} = + , {v}_{s} = - , {v}_{p} \leq {u}_{p} \}$$

$${E}_{L -} = \{ ( v , u ) \vert v ∈ {V}_{L , u} ∈ {V}_{sinks} , {v}_{c} = {u}_{c} , {v}_{s} = - , {v}_{s} = + , {v}_{p} \geq {u}_{p} \}$$

$${E}_{H +} = \{ ( v , u ) \vert v ∈ {V}_{s o urces} , u ∈ {V}_{H} , {v}_{c} = {u}_{c} , {v}_{s} = + , {v}_{s} = + , {v}_{p} \leq {u}_{p} \}$$

$${E}_{H -} = \{ ( v , u ) \vert v ∈ {V}_{s o urces} , u ∈ {V}_{H} , {v}_{c} = {u}_{c} , {v}_{s} = - , {v}_{s} = - , {v}_{p} \geq {u}_{p} \}$$

$${E}_{M} = {E}_{L +} ⋃ {E}_{L -} ⋃ {E}_{H +} ⋃ {E}_{H -}$$

The edge set E(Gₚ) can now be specified as:

$$E ( {G}_{p} ) = {E}_{WT} ⋃ {E}_{SV} ⋃ {E}_{M} \qquad ( 18 )$$

Let the weight of edge tuples in E(Gₚ) be defined as the genomic distance between each loci vertex, where connections between mate breakends have a distance of zero

$$w ( e ) = \{ \begin{matrix}\vert {e}_{{1}_{p}} - {e}_{{2}_{p}} \vert , & {e}_{2} ∈ {V}_{sink} \\ 0 , & {e}_{2} ∈ {V}_{source}\end{matrix} \qquad ( equation 19 )$$

Together V(Gₚ), E(Gₚ), and w:e→N₀ fully define Gₚ. This RNA-SV graph was built in python using the networkx package [38], and Dijkstra's algorithm was used to find the shortest weighted genomic path between every m_{L} to m_{H} chimeric intron vertices through an alternating set of sink and source breakend vertices. The genomic paths of each chimeric intron were appended in the order of appearance in each path p to produce a contig starting at the first chimeric intron start anchor and ending at the final chimeric intron end anchor. The contigs specified by the set of these shortest chimeric intron paths were padded at the beginning and end by prepending/appending enough sequence to encompass the full chimeric RNA alignment at the start/end of the contig and any annotated genes overlapping these start/end alignments. The set of all contigs identified through this procedure for all alignment paths arising from all chimeric reads for a given sample were combined with the set of contigs produced through the RNA-naive approach. This set of contigs was collapsed by removing all contigs whose sequence was a strict subset of another. This set of non-redundant contigs were appended to the tumor specific reference genome.

Small variants predicted to lead to NOPs were also used as a basis for tumor-specific contig construction. To identify all indels possibly leading to NOPs, indels within the bounds of protein coding genes were identified. If the indel was within the exonic boundaries of any protein coding exon, it was selected for inclusion in variants used for reconstruction. If the indel was in a non-protein coding region of the gene such as an intron or UTR, the variant was included if there was at least one long RNA read which covered the indel locus. Stoploss variants were identified by selecting variants which disrupted an annotated known stop codon. Mutations leading to novel splice junctions as described in 4.5.2 were also selected for inclusion in the reconstruction. A portion of the reference chromosome containing each variant was extracted to include entire region of any genes and/or long reads overlapping each variant position. The genomic change specified by each small variant was then performed on this contig with each variant producing a contig which was appended to the tumor-specific reference genome.

### 4.5.2 Novel Splice Junction Identification

Short read RNA splice junctions were considered novel and tumor specific if they were absent in the healthy tissues sequenced as part of the GTEx database [39] and were associated with a predicted causal somatic variant. The pre-compiled STAR splice junctions for GTEx v6 were downloaded from the Recount2 webserver and used as the normal tissue splice junction database [40]. Two general classes of variants were considered as causing novel splice junctions. In the first case, a variant is near an un-annotated splice site of the splice junction. These splice-gain variants are known to often lead to the formation of more-canonical splicing signals [17]. The second class of splice causing variants disrupt annotated splice sites by changing the genomic context of an annotated splice donor or acceptor. This splice site disruption may lead to full exon skipping or partial intron retention/truncation. The effect zone of these splice-disrupting variants was therefore taken as the 5′ start of the exon before the variant-affected exon up through the 3′ end of the exon after the variant-affected exon, including intronic regions. Any tumor specific splice junction with splice points within this genomic range was considered caused in cis by the splice-disrupting variant.

### 4.5.3 Tumor-Specific RNA Isoform Identification

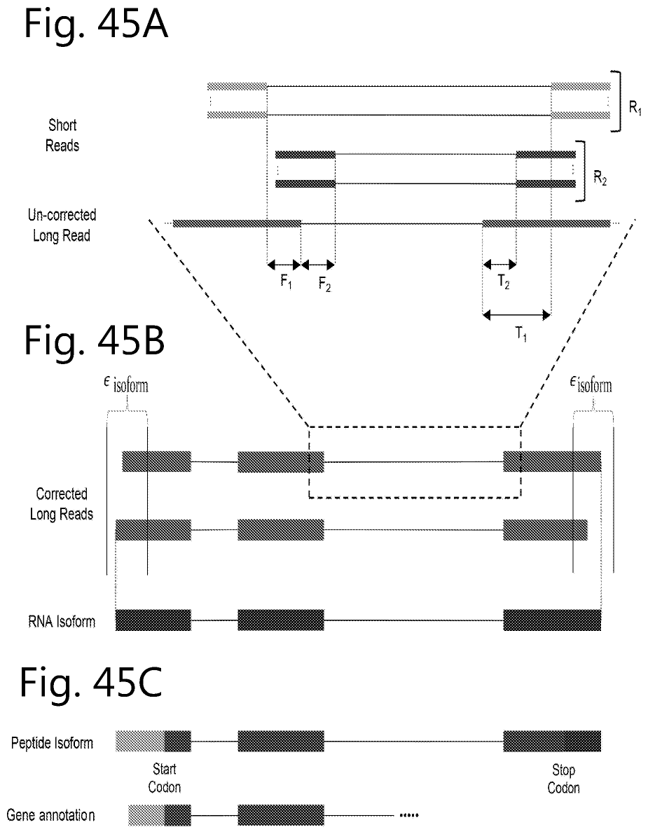

After alignment to the reconstructed tumor genome, tumor-specific RNA isoforms were identified through a combination of high-accuracy short reads and long but error prone long reads. Short read junctions were used to correct the splice points of long read alignments via a novel Bayesian splice-correction model illustrated in FIG. 45. Long read splice sites were corrected to short read splice sites using short read junctions in which both the 5′ and 3′ splice sites were within a ϵ_{splice} basepair (default 15) window of the respective long read splice sites. For cases in which multiple short read junctions satisfied this criteria for a given long read junction, the most likely short read junction was chosen via a Bayesian model in which the posterior probability that an observed long read junction arose from an mRNA with a given short read unction was calculated according to:

$$P ( {s}_{i} \vert {F}_{i} , {T}_{i} ) = \frac{P ( {F}_{i} , {T}_{i} \vert {s}_{i} ) P ( {s}_{i} )}{P ( {F}_{i} , {T}_{i} )} \qquad ( 20 )$$

where the event sᵢ is the long read arising from the splice junction i, and the event Fᵢ,Tᵢ is the observation of a long read having a given 5′ or 3′ distance pair from its underlying original splice sites. The prior probability that a long read arose from an RNA molecule with splice junction i was calculated according to:

$$P ( {s}_{i} ) = \frac{{R}_{i}}{R} \qquad ( 21 )$$

where Rᵢ is the number of short reads supporting junction i and R is the total spliced reads within the long read splice site window. The probability of observing the splice offset pair Fᵢ,Tᵢ given that the long read arose from an RNA molecule with splice junction i was calculated according to:

$$P ( {F}_{i} , {T}_{i} \vert {s}_{i} ) = \frac{{N}_{{F}_{i} {T}_{i}}}{N} \qquad ( 22 )$$

where N_{FiTi} is the number of times the given offset pair occurred in all other long read splice junction corrections which were unambiguous because a single short read junction was present within the correction window and N is the total number of unambiguously corrected junctions. Both N_{FiTi} and N were calculated for each sample based mapping of the short and long RNA to the base reference genome. The total probability of observing the long-read offset pair Fᵢ,Tᵢ irrespective of any given short read junction can be calculated according to:

$$P ( {F}_{i} , {T}_{i} ) = {\sum }_{j = 1}^{n} P ( {F}_{i} , {T}_{i} \vert {s}_{j} ) P ( {s}_{j} \qquad ( 23 )$$

where the summation is taken over the n splice junctions within the long-read junction window. Combining these expression gives:

$$P ( {F}_{i} , {T}_{i} ) = \frac{{N}_{{F}_{i} {T}_{i}} {R}_{i}}{{\sum }_{j = 1}^{n} {N}_{{F}_{j} {T}_{j}} {R}_{j}} \qquad ( 24 )$$

Splice junctions with the highest probability were chosen, and long read splice junctions for which no short read junctions had a correction probability of at least p_{splice} (default 0.9) were considered uncorrected. Reads which had one or more uncorrected junctions were not considered further for isoform identification. Splice corrected long read tumor-genome alignments were collapsed into RNA isoform structures by grouping reads with identical splice junctions together if their start loci and end loci were within ϵ_{isoform} basepairs (default 10) of each other.

### 4.5.4 Translation Prediction

Known protein coding transcript structures were used to predict the translation start sites of RNA isoforms. ENSEMBL gene annotations were parsed using the pyensembl python package [41]. These annotations were transposed onto the reconstructed tumor reference genome. For each RNA isoform, the set of most consistent transcript structures were identified by selecting the structures which had the most contiguous matching splice junctions, starting from the most 5′ transcript splice site. If a unique translation start site overlapping the RNA isoform could be identified for this collection of transcript structures, the protein sequence of the RNA isoform was predicted. If more than one translation start site was consistent with the transcript structure, the protein sequence of the isoform was considered ambiguous and a translation prediction was not performed. If the most consistent transcript structure was of a non-coding biotype, the RNA isoform was annotated as non-coding.

### 4.5.5 NOP Identification

Once full-length protein isoforms arising from RNA aligned to the reconstructed reference genome were identified, the tumor-specific portions of each peptide were annotated as NOPs. Each amino acid of each protein coding isoform was annotated as novel or WT based on the following set of criteria, and strings of consecutive novel amino acids were considered distinct NOPs. For an amino acid to be considered novel in this protocol it must:
1. not overlap in-frame with a known WT protein coding isoform
2. be a part of at least one 8, 9, 10, or 11-mer amino acid sequence which is not in the set of known WT peptides
3. arise from a position in the RNA isoform which is downstream of the first potentially causal variant position

The first criteria is satisfied if the first nucleotide of the amino acid's codon does not align to a genomic position which is a known WT P-site. To rapidly check this for each amino acid in each protein isoform, a P-site genome was pre-compiled by annotating each position of each reference chromosome as either not overlapping with any known P-site, overlapping a P-site in the sense strand, overlapping a P-site in the antisense strand, or overlapping in both strands. Pyensembl [41] with ENSEMBL reference version 75 (GRch37) was used to determine the P-site status of each position in the reference genome. This P-site genome was compiled in a coded string format and stored as a fasta file which was loaded for each sample. This format can easily be extended to include other gene references or WT P-sites from other sources such as RiboSeq experiments.

While not overlapping with a WT P-site indicates a novel portion of the genome is being translated, homology between the novel translated region and other normally translated regions of the genome can mean a portion of an otherwise novel protein isoform may be identical to known WT proteins. To avoid considering these portions as part of NOPs, each amino acid must be a part of at least one k-mer which is not present in the set of known WT peptides to be considered novel. As NOPs represent potentially interesting neoantigen targets, the k-mer sizes corresponding to potential MHC-I epitopes were chosen. A pre-compiled WT k-mer database was compiled by decomposing all peptides in ENSEMBL and RefSeq protein databases into all possible 8-11mers. This set was made unique and stored as a flat file which was loaded as a set for each sample run. For each amino acid in each isoform, all possible 8-11mers which contained the amino acid in that peptide (max 38) were screened against the WT k-mer set. If all of the 8-11mers were contained within the WT set, the amino acid was not considered novel.

An amino acid must also arise from a codon which is downstream of the first variant which would potentially be driving tumor specific translation. For indels, stoploss, and structural variants the amino acid must simply be downstream of the first variant spanned by the RNA isoform. For splice NOPs, the amino acid must be downstream of the first novel splice junction. To avoid considering amino acids novel due to likely un-annotated splice isoforms which are not altered by the underlying somatic variants, the first exon downstream of the first novel splice junction must contain at least one novel amino acid for any of the amino acids in the peptide isoform to be considered novel. Additionally, amino acids in peptides spanning SVs are not considered novel if they are within the boundary of the anchor gene which is driving translation.

### 4.6 MHC-Binding Prediction

Polysolver [42] was used to predict HLA types using WGS data. NetMHCpan4.1 [43] was used to predict MHC-binding using an EL score cutoff of 2 for binders.

### 4.7 Self Similarity

Self similarity of epitopes was computed as described in [29]. As a normal reference the ENSEMBL GRCH38 proteome was used. To generate random epitopes, random strings of 1000 nucleotides were generated with a GC content of 40.9% to match the bias of the human genome [44]. These NT strings were translated and a random 9-mer epitope was selected from the collection of resultant 9-mers. This process was repeated until enough random epitopes were generated to match the number of NOP epitopes.

### 4.8 In-Silico Vaccine Design

To construct patient specific framome vaccine designs of a given amino acid length the longest NOPs were chained together with the remainder of the vaccine consisting of a NOP portion. Missense vaccines of a given length were constructed by chaining together 21 amino acid long sequences with the variant amino acid in the center. Any remaining required length consisted of an amino acid sequence of the required length with the missense mutation in the middle to provide the most potential CD8 epitopes. For both classes the minimum amount of amino acids appended was 8.

### 4.9 RiboSeq Analysis

RiboSeq data for human cancer cell lines A375, MCF-7 and 7860 were generated as previously described. Data were mapped to human reference GRCh37. Ribosomal P-site offsets were calculated using the RiboSeQC R package. Long-read Nanopore RNA sequencing was performed on A375 cells. Short-read RNA reads (SRA accession number SRR8616020) and SV calls were obtained from the CCLE [45]. SV calls were converted to breakend format. The FramePro pipeline was then used to identify all neo-ORFs and corresponding NOPs for this cell line. RiboSeq read mapping locations (P-sites) were intersected with the portions of neo-ORF long-read RNA mappings leading to hidden frame NOPs. The periodicity of RiboSeq read P-site coverage in these regions was identified using custom scripts.

### 4.10 Tumor Suppressor Genes

NOPs were classified as arising from tumor suppressor genes if their gene of origin was in the TSGene database [46].

### 4.11 In Vitro MHC Binding and Tetramer Staining

Selected epitopes for the assessment of in vitro binding were synthesized by GeneCust (GeneCustance). In vitro binding was performed as described previously [47]. Briefly, a conditional HLA class I complex is stabilized through a photolabile peptide, which can be dissociated through UV irradiation. If the cleavage occurred in the presence of another HLA class I peptide, the reaction resulted in net exchange of the cleaved peptide, yielding an HLA class I complex with an epitope of choice. The peptide exchange efficiency was then analyzed using an HLA class I ELISA. The combined technologies allowed the identification of ligands for an HLA class I molecule of interest. HLA-peptide complexes with binding affinity >40% were then used to prepare fluorescently labeled tetramers for combinatorial coding and phenotyping, as described before [48].

### REFERENCES

[1] Robert, C. A decade of immune-checkpoint inhibitors in cancer therapy. Nature Communications 11, 1-3 (2020).
[2] Schumacher, T. N., Scheper, W. & Kvistborg, P. Cancer neoantigens. Annual review of immunology 37, 173-200 (2019).
[3] Blass, E. & Ott, P. A. Advances in the development of personalized neoantigen-based therapeutic cancer vaccines. Nature Reviews Clinical Oncology 18, 215-229 (2021).
[4] Richters, M. M. et al. Best practices for bioinformatic characterization of neoantigens for clinical utility. Genome medicine 11, 1-21 (2019).
[5] Shemesh, C. S. et al. Personalized cancer vaccines: clinical landscape, challenges, and opportunities. Molecular Therapy 29, 555-570 (2021).
[6] Lee, M. Y., Jeon, J. W., Sievers, C. & Allen, C. T. Antigen processing and presentation in cancer immunotherapy. Journal for immunotherapy of cancer 8 (2020).
[7] Garcia-Garijo, A., Fajardo, C. A. & Gros, A. Determinants for neoantigen identification. Frontiers in immunology 10, 1392 (2019).
[8] Priestley, P. et al. Pan-cancer whole-genome analyses of metastatic solid tumours. Nature 575, 210-216 (2019).
[9] Turajlic, S. et al. Insertion-and-deletion-derived tumour-specific neoantigens and the immunogenic phenotype: a pan-cancer analysis. The lancet oncology 18, 1009-1021 (2017).
[10] Roudko, V. et al. Shared immunogenic poly-epitope frameshift mutations in microsatellite unstable tumors. Cell 183, 1634-1649 (2020).
[11] Koster, J. & Plasterk, R. H. A library of neo open reading frame peptides (nops) as a sustainable resource of common neoantigens in up to 50% of cancer patients. Scientific reports 9, 1-8 (2019).
[12] Rathe, S. K. et al. Identification of candidate neoantigens produced by fusion transcripts in human osteosarcomas. Scientific reports 9, 1-11 (2019).
[13] Fotakis, G., Rieder, D., Haider, M., Trajanoski, Z. & Finotello, F. Neofuse: predicting fusion neoantigens from rna sequencing data. Bioinformatics 36, 2260-2261 (2020).
[14] Yang, W. et al. Immunogenic neoantigens derived from gene fusions stimulate t cell responses. Nature medicine 25, 767-775 (2019).
[15] Mansfield, A. S. et al. Neoantigenic potential of complex chromosomal rearrangements in mesothelioma. Journal of Thoracic Oncology 14, 276-287 (2019).
[16] Kosari, F. et al. Tumor junction burden and antigen presentation as predictors of survival in mesothelioma treated with immune checkpoint inhibitors. Journal of Thoracic Oncology (2021).
[17] Jung, H., Lee, K. S. & Choi, J. K. Comprehensive characterisation of intronic mis-splicing mutations in human cancers. Oncogene 40, 1347-1361 (2021).
[18] Jayasinghe, R. G. et al. Systematic analysis of splice-site-creating mutations in cancer. Cell reports 23, 270-281 (2018).
[19] Shiraishi, Y. et al. A comprehensive characterization of cis-acting splicing-associated variants in human cancer. Genome research 28, 1111-1125 (2018).
[20] Dhamija, S. et al. A pan-cancer analysis reveals nonstop extension mutations causing smad4 tumour suppressor degradation. Nature cell biology 22, 999-1010 (2020).
[21] Ott, P. A. et al. A phase ib trial of personalized neoantigen therapy plus anti-pd-1 in patients with advanced melanoma, non-small cell lung cancer, or bladder cancer. Cell 183, 347-362 (2020).
[22] Ott, P. A. et al. An immunogenic personal neoantigen vaccine for patients with melanoma. Nature 547, 217-221 (2017).
[23] Sahin, U. et al. Personalized rna mutanome vaccines mobilize poly-specific therapeutic immunity against cancer. Nature 547, 222-226 (2017).
[24] Mertens, F., Johansson, B., Fioretos, T. & Mitelman, F. The emerging complexity of gene fusions in cancer. Nature Reviews Cancer 15, 371-381 (2015).
[25] McGranahan, N. et al. Clonal neoantigens elicit t cell immunoreactivity and sensitivity to immune checkpoint blockade. Science 351, 1463-1469 (2016).
[26] Westcott, P. M. et al. Low neoantigen expression and poor t-cell priming underlie early immune escape in colorectal cancer. Nature cancer 2, 1071-1085 (2021).
[27] Litchfield, K. et al. Escape from nonsense-mediated decay associates with anti-tumor immunogenicity. Nature communications 11, 1-11 (2020).
[28] Cort'es-Ciriano, I. et al. Comprehensive analysis of chromothripsis in 2,658 human cancers using wholegenome sequencing. Nature genetics 52, 331-341 (2020).
[29] Wood, M. A. et al. Population-level distribution and putative immunogenicity of cancer neoepitopes. BMC cancer 18, 1-15 (2018).
[30] Klempner, S. J. et al. Tumor mutational burden as a predictive biomarker for response to immune checkpoint inhibitors: a review of current evidence. The oncologist 25, e147 (2020).
[31] Rizvi, N. A. et al. Mutational landscape determines sensitivity to pd-1 blockade in non-small cell lung cancer. Science 348, 124-128 (2015).
[32] Smart, A. C. et al. Intron retention is a source of neoepitopes in cancer. Nature biotechnology 36, 1056-1058 (2018).
[33] Kahles, A. et al. Comprehensive analysis of alternative splicing across tumors from 8,705 patients. Cancer cell 34, 211-224 (2018).
[34] Gebert, J. et al. Recurrent frameshift neoantigen vaccine elicits protective immunity with reduced tumor burden and improved overall survival in a lynch syndrome mouse model. Gastroenterology 161, 1288-1302 (2021).
[35] Li, Y. et al. Patterns of somatic structural variation in human cancer genomes. Nature 578, 112-121 (2020).
[36] De Paoli-Iseppi, R., Gleeson, J. & Clark, M. B. Isoform age-splice isoform profiling using long-read technologies. Frontiers in Molecular Biosciences 8 (2021).
[37] Di Tommaso, P. et al. Nextflow enables reproducible computational workflows. Nature biotechnology 35, 316-319 (2017).
[38] Hagberg, A., Swart, P. & S Chult, D. Exploring network structure, dynamics, and function using networkx. Tech. Rep., Los Alamos National Lab. (LANL), Los Alamos, NM (United States) (2008).
[39] Consortium, G. et al. The genotype-tissue expression (gtex) pilot analysis: Multitissue gene regulation in humans. Science 348, 648-660 (2015).
[40] Leonardo, C.-T., Abhinav, N. & Kai, K. Ellis shannon e, taub margaret a, hansen kasper d, jaffe andrew e, langmead ben, leek jeffrey t. reproducible rna-seq analysis using recount2. Nature Biotechnology 35, 319-321 (2017).
[41] Rubinsteyn, A. et al. Computational pipeline for the pgv-001 neoantigen vaccine trial. Frontiers in immunology 8, 1807 (2018).
[42] Shukla, S. A. et al. Comprehensive analysis of cancer-associated somatic mutations in class i hla genes. Nature biotechnology 33, 1152-1158 (2015).
[43] Reynisson, B., Alvarez, B., Paul, S., Peters, B. & Nielsen, M. Netmhcpan-4.1 and netmhciipan-4.0: improved predictions of mhc antigen presentation by concurrent motif deconvolution and integration of ms mhc eluted ligand data. Nucleic acids research 48, W449-W454 (2020).
[44] Piovesan, A. et al. On the length, weight and gc content of the human genome. BMC research notes 12, 1-7 (2019).
[45] Ghandi, M. et al. Next-generation characterization of the cancer cell line encyclopedia. Nature 569, 503-508 (2019).
[46] Zhao, M., Kim, P., Mitra, R., Zhao, J. & Zhao, Z. Tsgene 2.0: an updated literature-based knowledgebase for tumor suppressor genes. Nucleic acids research 44, D1023-D1031 (2016).
[47] Rodenko, B. et al. Generation of peptide-mhc class i complexes through uv-mediated ligand exchange. Nature protocols 1, 1120-1132 (2006).
[48] Hadrup, S. R. et al. Parallel detection of antigen-specific t-cell responses by multidimensional encoding of mhc multimers. Nature methods 6, 520-526 (2009).

### Example 8 Identification of Memory T-Cells in the Peripheral Blood of a Patient with Cancer

To characterize the immunogenic properties of tumor-specific neo-open reading frames derived from splice mutations, the affinity of epitopes to various HLA-A and HLA-B alleles derived from a splice mutation-derived neo-open reading frame peptide will be assessed by in vitro binding assays. First, epitopes are selected for a splice neo-open reading frame peptide identified in a patient with lung cancer. Epitopes are selected by performing HLA affinity prediction for each of the HLA alleles in the patient and only epitopes with highest affinity were selected (i.e. EL score below 2), as described (Reynisson, B. et al, Nucleic acids research 48, W449-W454 (2020)). Epitopes are synthesized and in vitro binding will be performed (Rodenko, B. et al. Nature protocols 1, 1120-1132 (2006)). This will reveal several epitopes binding to the HLA-A and HLA-B alleles specific for this patient.

Next, fluorescently labeled HLA tetramers are generated each carrying an epitope with at least 40% binding affinity, as determined by the in vitro binding measurements. The tetramer-epitope complexes are subsequently used to stain CD8+ T-cells present in the peripheral blood mononuclear cell fraction of the patient using combinatorial coding (Hadrup, S. R. et al. Nature methods 6, 520-526 (2009)).

CD8+ T-cells binding to specific HLA tetramer-epitope complexes are phenotyped to evaluate if they have been exposed to the antigen already. This analysis will show that memory T-cells exist (i.e. CD8+CD45RA−, CD27−/dim) in the blood of the patient with specificity to one of the epitopes derived from the splice neo-open reading frame peptide. We conclude that epitopes derived from splice neo-open reading frame peptides can bind to HLA-A and HLA-B alleles expressed in a patient, and that antigen-specific immune responses can be induced by such epitopes.

In a subsequent experiment, the immunogenic properties of the same splice neo-open reading frame peptide are determined using in vitro immunogenicity assays. Therefore, monocyte-derived immature dendritic cells are generated from peripheral blood mononuclear cells obtained from healthy donors with various HLA types. The dendritic cells are electroporated with an mRNA construct encoding the splice neo-open reading frame. Following electroporation and maturation, the DCs are co-cultured with Pan T cells. Pan T cells are re-stimulated with transfected dendritic cells and subsequently harvested and seeded onto IFN-gamma FluoroSpot plates for read-out. FluoroSpots spot forming units will be recorded and compared to negative control (no antigen) and positive control (viral antigens). This experiment provides a broad view on the capacity of the splice neo-open reading frame peptide to trigger T-cell mediated IFN-gamma production for a large number of donors across different HLA alleles.

1. A method for identifying neoantigen sequences, said method comprising:
i) performing whole genome sequencing of at least one tumor sample and at least one healthy sample from an individual,
ii) performing long-read RNA sequencing on RNA or long-read sequencing on the corresponding cDNA from the at least one tumor sample;
iii) identifying somatic genomic changes in nucleic acid sequences from at least one tumor sample from an individual, said step comprising determining the presence of single nucleotide variants (SNVs), indels, and structural variants that result in tumor specific open reading frames,
wherein said step comprises:
determining the presence of cis-splicing mutations that result in tumor specific open reading frames;
determining the presence of intragenic frameshift mutations in polypeptide encoding sequences, wherein the mutation results in a tumor specific open reading frame,
determining the presence of DNA rearrangements resulting in new junctions of DNA sequences, wherein the DNA rearrangement results in a tumor specific open reading frame, and
determining the presence of a mutation in a stop codon, wherein the mutation results in a tumor specific open reading frame;
iv) determining the predicted amino acid sequences encoded by the tumor specific open reading frames, and
v) selecting, as candidate neoantigen peptide sequences, amino acid sequences comprising at least 8, preferably at least 9, amino acids, wherein the neoantigen peptide sequences comprise at least one amino acid, preferably at least 4 contiguous amino acids, encoded by a tumor specific open reading frame.

2. The method of claim 1, wherein step i) comprises performing long-read whole genome sequencing of the at least one tumor sample and at least one healthy sample from the individual.

3. The method of any one of the preceding claims, comprising performing long-read RNA sequencing on RNA or long-read sequencing on the corresponding cDNA from at least one tumor sample, wherein the RNA is poly-(A) selected mRNA and/or 5′ cap containing mRNA, preferably wherein the poly-(A) and/or 5′ cap containing mRNA is selected by a purification step.

4. The method of any one of the preceding claims, wherein the RNA sequencing is performed using long-read direct RNA sequencing, preferably Nanopore sequencing, or long-read cDNA sequencing.

5. The method of any one of the preceding claims, further comprising performing short-read RNA sequencing on RNA or short-read sequencing on the corresponding cDNA from at least one tumor sample.

6. The method of any one of the preceding claims, further comprising performing consensus sequencing on RNA or the corresponding cDNA from at least one tumor sample, preferably wherein the RNA is poly-(A) selected mRNA and/or 5′ cap containing mRNA.

7. The method of any one of the preceding claims, wherein the method further comprises selecting poly-(A) mRNA from said tumor sample and performing long-read RNA sequencing or long-read cDNA sequencing based on the poly-(A) selected mRNA.

8. The method of claim 7, wherein the method further comprises selecting 5′ cap containing mRNA from said tumor sample and performing long-read RNA sequencing or long-read cDNA sequencing based on the selected mRNA.

9. The method of any of the preceding claims, wherein the selected candidate neoantigen peptide sequences comprise amino acid sequences resulting from cis-splicing mutations that result in tumor specific open reading frames, preferably wherein the method further comprises comparing the splice junction resulting from the cis-splicing mutation with a database of mRNA wild-type splice junctions, and selecting as candidate neoantigen peptide sequences those sequences where said splice junction is not present in the database of mRNA wild-type splice junctions.

10. The method of any of the preceding claims, wherein the selected candidate neoantigen peptide sequences comprise amino acid sequences resulting from:
intragenic frameshift mutations in polypeptide encoding sequences that result in tumor specific open reading frames;
DNA rearrangements resulting in new junctions of DNA sequences, wherein the DNA rearrangement results in a tumor specific open reading frame; and/or
mutation in a stop codon, wherein the mutation results in a tumor specific open reading frame.

11. The method of any of the preceding claims, wherein said method comprises defining tumor specific open reading frames by determining strings of one or more consecutive tumor specific amino acids, where an amino acid is considered tumor specific if
(i) the position of the first nucleotide of the triplet encoding the amino acid does not align to a genomic position which is a known wild-type P-site;
(ii) the amino acid is part of at least one k-mer amino acid sequence which does not correspond to a known wild-type human peptide, wherein k is at least 8, preferably 8, 9, 10, or 11; and
(iii) the amino acid is encoded by a genomic sequence that is downstream of the somatic genomic change, wherein for a cis-splicing mutation each amino acid of said string of one or more consecutive novel amino acids is encoded by a genomic sequence that is downstream of the first novel splice junction.

12. The method of any of the preceding claims, wherein the method comprises selecting neoantigen peptide sequences having one or more of the following characteristics:
neoantigen peptide sequences which do not share a contiguous stretch of at least 4 amino acids with human protein reference sequences;
neoantigen peptide sequences wherein the genomic variant allele frequency of the respective somatic mutation in the tumor cells of a tumor sample is at least 0.1;
neoantigen peptide sequences wherein the cysteine content for each peptide is 30% or less, where cysteine content (Qcys) is defined as the number of cysteines in said sequence divided by the total number of amino acids in said sequence;
neoantigen peptide sequences for which the underlying somatic mutations have a maximum distance with regard to chromosomal location, preferably wherein each mutation is located on a different chromosomal arm;
neoantigen peptide sequences wherein the peptides are predicted to comprise one or more MHC I and/or MHC II binding epitopes; and
neoantigen peptide sequences for which the RNA expression level of the underlying transcripts encoding such neoantigen peptide sequences have a gene expression value of at least 0.1 transcript per million (TPM) in the tumor sample.

13. The method of any of the preceding claims, comprising identifying candidate neoantigen sequences from a plurality of individuals and selecting as shared candidate neoantigen sequences, candidate neoantigen peptide sequences identified from at least two individuals.

14. A method for preparing a vaccine or collection of vaccines for the treatment of cancer in an individual, comprising identifying and selecting candidate neoantigen peptide sequences according to any of the preceding claims and preparing a vaccine or collection of vaccines comprising one or more peptides having said amino acid sequences or comprising one or more nucleic acid molecules encoding said amino acid sequences.

15. A method for preparing an antigen or a collection of antigens comprising identifying and selecting candidate neoantigen peptide amino acid sequences according to any of claims 1-13 and preparing an antigen or collection of antigens comprising one or more peptides having said amino acid sequences or comprising one or more nucleic acid molecules encoding said amino acid sequences.

16. The method of any one of claims 14-15, wherein said amino acid sequences encoded by the tumor specific open reading frames comprise at least 50 amino acids.

17. The method of any one of claims 14-16, wherein said vaccine, collection of vaccines, antigen, or collection of antigens, respectively, comprise or encode essentially all candidate neoantigen peptides identified.

18. The method of any one of claims 14-17, wherein said nucleic acid molecule or collection of nucleic acid molecules comprises deoxyribonucleic acid (DNA) and/or ribonucleic acid (RNA).

19. The method of claim 18, wherein said nucleic acid molecule is mRNA, self-amplifying RNA, circular RNA, or viral RNA.

20. The method of claim 18 or 19, additionally comprising a step of RNA in vitro transcription.

21. The method of claims 18 to 20, additionally comprising a step formulating the nucleic acid molecule or collection of nucleic acid molecules, preferably the RNA, in a lipid-based carrier, preferably wherein said lipid-based carrier is selected from lipid nanoparticles, liposomes, lipoplexes, and nanoliposomes.

22. A vaccine or collection of vaccines for the treatment of cancer, obtainable by a method according to any one of claim 14, or 16-21.

23. A peptide antigen or collection of peptide antigens obtainable by the method according to any one of claims 15-17.

24. An isolated nucleic acid molecule or collection of nucleic acid molecules that encode the peptide antigen or collection of peptide antigens of claim 23, preferably wherein the nucleic acid molecule or collection of nucleic acid molecules comprises deoxyribonucleic acid (DNA) and/or ribonucleic acid (RNA).

25. A peptide antigen obtainable by identifying candidate neoantigen peptide amino acid sequences according to any one of claims 1-13 and preparing a peptide comprising one or more of said neoantigen peptide amino acid sequences.

26. An isolated nucleic acid molecule encoding the peptide antigen of claim 25, preferably wherein the nucleic acid molecule or collection of nucleic acid molecules comprises deoxyribonucleic acid (DNA) and/or ribonucleic acid (RNA).

27. A pharmaceutical composition comprising
i) the nucleic acid molecule or collection of nucleic acid molecules from any one of claim 24 or 26, the one or more nucleic acid molecules obtainable by a method of any one of claims 14-20, the vaccine or collection of vaccines obtainable by a method according to any one of claims 14-21, and the vaccine or collection of vaccines according to claim 22; and comprising one or more nucleic acid molecules and
ii) a lipid-based carrier, preferably wherein said lipid-based carrier is selected from lipid nanoparticles, liposomes, lipoplexes, and nanoliposomes.

28. A binding molecule or collection of binding molecules that binds the peptide antigen according to claim 23 or 25 or the collection of peptide antigens according to claim 23, wherein the binding molecule is an antibody, a T-cell receptor, or an antigen binding fragment thereof.

29. A chimeric antigen receptor or collection of chimeric antigen receptors that binds the peptide antigen according to claim 23 or 25 or the collection of peptide antigens according to claim 23, wherein each chimeric antigen receptor comprises i) a T cell activation molecule; ii) a transmembrane region; and iii) an antigen recognition moiety.

30. One or more T-cells expressing the T-cell receptor or collection of T-cell receptors of claim 28 or the chimeric antigen receptor or collection of chimeric antigen receptors of claim 29.

31. The vaccine or collection of vaccines according to claim 22, the peptide antigen or collection of peptide antigens according to claim 23 or 25, the nucleic acid molecule or collection of nucleic acid molecules according to claim 24 or 26, the pharmaceutical composition of claim 27, the binding molecule or collection of binding molecules of claim 28, the T-cell receptor or collection of T-cell receptors of claim 28, the chimeric antigen receptor or collection of chimeric antigen receptors of claim 29, or the one or more T-cells of claim 30, for use in the treatment of cancer, preferably cancer in an individual.

32. A method for preparing a cellular immunotherapy for the treatment of cancer, said method comprising contacting T-cells with one or more candidate neoantigen peptide sequences identified from the individual according to any one of claims 1-13 to produce a cellular immunotherapy.

33. The method according to claim 32, further comprising selecting T-cells with specificity for one or more of said neoantigen peptide sequences.

34. The method according to claim 32 or 33, wherein said contacting results in the stimulation of the T-cells.

35. The method according to any one of claims 32-34, further comprising the in vitro expansion of stimulated and/or selected T-cells.

36. The method according to any one of claims 32-35, wherein the T-cells are obtained from said individual.

37. The method according to any one of claims 32-36, further comprising the identification of or sequencing of a T-cell receptor or a collection of T-cell receptors with specificity for one or more of said neoantigen peptide sequences.

38. The method according to any one of claims 32-37, wherein said contacting step comprises contacting T-cells with antigen-presenting cells transfected with one or more candidate neoantigen peptides or one or more nucleic acid molecules encoding the one or more candidate neoantigen peptides.

39. The method of claim 38, comprising transfecting T-cells with one or more nucleic acid molecules that encode for a T-cell receptor with specificity for one or more of said neoantigen peptide sequences.

40. A cellular immunotherapy for use in the treatment of cancer, preferably cancer in an individual, wherein said cellular immunotherapy comprises the administration of T-cells prepared according to a method of any one of claims 32-39.

41. A method of treating cancer, preferably cancer in an individual, the method comprising
i) performing whole genome sequencing of a tumor sample and a healthy sample from an individual in need thereof,
ii) performing long-read RNA sequencing on RNA or long-read sequencing on the corresponding cDNA from at least one tumor sample;
iii) identifying somatic genomic changes in nucleic acid sequences from at least one tumor sample from an individual, said step comprising determining the presence of single nucleotide variants (SNVs), indels, and structural variants that result in tumor specific open reading frames,
wherein said step comprises:
determining the presence of cis-splicing mutations that result in tumor specific open reading frames;
determining the presence of intragenic frameshift mutations in polypeptide encoding sequences, wherein the mutation results in a tumor specific open reading frame,
determining the presence of DNA rearrangements resulting in new junctions of DNA sequences, wherein the DNA rearrangement results in a tumor specific open reading frame, and
determining the presence of a mutation in a stop codon, wherein the mutation results in a tumor specific open reading frame;
iv) determining the predicted amino acid sequences encoded by the tumor specific open reading frames,
v) selecting, as candidate neoantigen peptide sequences, amino acid sequences comprising at least 8 amino acids, wherein the neoantigen peptide sequences comprise at least one amino acid encoded by a tumor specific open reading frame, and
vi) administering to said individual
a peptide antigen or a collection of peptide antigens comprising at least one of said candidate neoantigen peptide sequences,
one or more nucleic acid molecules encoding at least one of said candidate neoantigen peptide sequences,
one or more T-cells expressing T-cell receptors or chimeric antigen receptors with specificity for at least one of said candidate neoantigen peptide sequences.
